SEQUENCE LISTING (65 sequences)

SEQ ID NO: 1 - protein, 559 aa, Oryza sativa
MASITNWLGFSSSSFSGAGADPVLPHPPLQEWGSAYEGGGTVAAAGGEETAAPKLEDFLGMQVQQETAAAAAGHGRGGSSSVVGLSMIKNWLRSQPPPAVVGGEDAMMALAVSTSASPPVDATVPACISPDGMGSKAADGGGAAEAAAAAAAQRMKAAMDTFGQRTSIYRGVTKHRWTGRYEAHLWDNSCRREGQTRKGRQVNAGGYDKEEKAARAYDLAALKYWGTTTTTNFPVSNYEKELDEMKHMNRQEFVASLRRKSSGFSRGASIYRGVTRHHQHGRWQARIGRVAGNKDLYLGTFGTQEEAAEAYDIAAIKFRGLNAVTNFDMSRYDVKSIIESSNLPIGTGTTRRLKDSSDHTDNVMDINVNTEPNNVVSSHFTNGVGNYGSQHYGYNGWSPISMQPIPSQYANGQPRAWLKQEQDSSVVTAAQNLHNLHHFSSLGYTHNFFQQSDVPDVTGFVDAPSRSSDSYSFRYNGTNGFHGLPGGISYAMPVATAVDQGQGIHGYGEDGVAGIDTTHDLYGSRNVYYLSEGSLLADVEKEGDYGQSVGGNSWVLPTP

SEQ ID NO: 2 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNNINNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGTTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 3 - protein, 579 aa, Brassica napus
MNNNWLGFSLSPYEQNHHRKDVYSSTTTTVVDVAGEYCYDPTAASDESSAIQTSFPSPFGVVVDAFTRDNNSHSRDWDINGCACNNIHNDEQDGPKLENFLGRTTTIYNTNENVGDGSGSGCYGGGDGGGGSLGLSMIKTWLRNQPVDNVDNQENGNAAKGLSLSMNSSTSCDNNNDSNNNVVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEVEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENSASGWQNAAVQHHQGVDLSLLHQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQQSPGGDFPAAMTNNVGSNMYYHGEGGGEVAPTFTVWNDN

SEQ ID NO: 4 - protein, 579 aa, Brassica napus
MNNNWLGFSLSPYEQNHHRKDVCSSTTTTAVDVAGEYCYDPTAASDESSAIQTSFPSPFGVVLDAFTRDNNSHSRDWDINGSACNNIHNDEQDGPKLENFLGRTTTIYNTNENVGDIDGSGCYGGGDGGGGSLGLSMIKTWLRNQPVDNVDNQENGNGAKGLSLSMNSSTSCDNNNYSSNNLVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEIEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENNASGWQNAAVQHHQGVDLSLLQQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQHSPGGDFPAAMTNNVGSNMYYHGEGGGEVAPTFTVWNDN

SEQ ID NO: 5 - protein, 559 aa, Oryza sativa
MASITNWLGFSSSSFSGAGADPVLPHPPLQEWGSAYEGGGTVAAAGGEETAAPKLEDFLGMQVQQETAAAAAGHGRGGSSSVVGLSMIKNWLRSQPPPAVVGGEDAMMALAVSTSASPPVDATVPACISPDGMGSKAADGGGAAEAAAAAAAQRMKAAMDTFGQRTSIYRGVTKHRWTGRYEAHLWDNSCRREGQTRKGRQVYLGGYDKEEKAARAYDLAALKYWGTTTTTNFPVSNYEKELDEMKHMNRQEFVASLRRKSSGFSRGASIYRGVTRHHQHGRWQARIGRVAGNKDLYLGTFGTQEEAAEAYDIAAIKFRGLNAVTNFDMSRYDVKSIIESSNLPIGTGTTRRLKDSSDHTDNVMDINVNTEPNNVVSSHFTNGVGNYGSQHYGYNGWSPISMQPIPSQYANGQPRAWLKQEQDSSVVTAAQNLHNLHHFSSLGYTHNFFQQSDVPDVTGFVDAPSRSSDSYSFRYNGTNGFHGLPGGISYAMPVATAVDQGQGIHGYGEDGVAGIDTTHDLYGSRNVYYLSEGSLLADVEKEGDYGQSVGGNSWVLPTP

SEQ ID NO: 6 - protein, 695 aa, Oryza sativa
MATMNNWLAFSLSPQDQLPPSQTNSTLISAAATTTTAGDSSTGDVCFNIPQDWSMRGSELSALVAEPKLEDFLGGISFSEQQHHHGGKGGVIPSSAAACYASSGSSVGYLYPPPSSSSLQFADSVMVATSSPVVAHDGVSGGGMVSAAAAAAASGNGGIGLSMIKNWLRSQPAPQPAQALSLSMNMAGTTTAQGGGAMALLAGAGERGRTTPASESLSTSAHGATTATMAGGRKEINEEGSGSAGAVVAVGSESGGSGAVVEAGAAAAAARKSVDTFGQRTSIYRGVTRHRWTGRYEAHLWDNSCRREGQTRKGRQVYLGGYDKEEKAARAYDLAALKYWGPTTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSAALPVGTAAKRLKDAEAAAAYDVGRIASHLGGDGAYAAHYGHHHHSAAAAWPTIAFQAAAAPPPHAAGLYHPYAQPLRGWCKQEQDHAVIAAAHSLQDLHHLNLGAAAAAHDFFSQAMQQQHGLGSIDNASLEHSTGSNSVVYNGDNGGGGGGYIMAPMSAVSATATAVASSHDHGGDGGKQVQMGYDSYLVGADAYGGGGAGRMPSWAMTPASAPAATSSSDMTGVCHGAQLFSVWNDT

SEQ ID NO: 7 - protein, 679 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVPSGAASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 8 - protein, 707 aa, Glycine max
MGSMNLLGFSLSPHEEHPSSQDHSQTTPSRFSFNPDGSISSTDVAGGCFDLTSDSTPHLLNLPSYGIYEAFHRNNSINTTQDWKENYNSQNLLLGTSCNKQNMNQNQQQQPKLENFLGGHSFGEHEQTYGGNSASTDYMFPAQPVSAGGGGSGGGSNNNNNSNSIGLSMIKTWLRNQPPNSENINNNNESGGNIRSSVQQTLSLSMSTGSQSSTSLPLLTASVDNGESPSDNKQPNTSAALDSTQTGAIETAPRKSIDTFGQRTSIYRGVTRHRWTGRYEAHLWDNSCRREGQTRKGRQVYLGGYDKEEKAARAYDLAALKYWGTTTTTNFPISHYEKELEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKSILESTTLPIGGAAKRLKDMEQVELSVDNGHRADQVDHSIIMSSHLTQGINNNYAGGGTATHHNWHNAHAFHQPQPCTTMHYPYGQRINWCKQEQQDNSDAPHSLSYSDIHQLQLGNNGTHNFFHTNSGLHPMLSMDSASIDNSSSSNSVVYDGYGGGGGYNVMPMGTTTAVVASDGDQNPRSNHGFGDNEIKALGYESVYGSATDSYHAHARNLYYLTQQQSSSVDTVKASAYDQGSACNTWVPTAIPTHAPRSTTSMALCHGATTPFSLLHE

SEQ ID NO: 9 - protein, 649 aa, Capsicum annuum
MKMKSMNDDSSSSNNSNSNNNNHSSAATNSNNWLGFSLSPHMKMEVTNASETQQQQHPHQQQFAQSFYLSSSPTTMNVSTASALCYENNPFHSSLSVMPLKSDGSLCIMEALSRSHADAMVQSSSPKLEDFLGGASQYGSHEREAMALSLDSLYYHQNDEDIQVHSHHPYYSPMHCHGMYQESLLEETKPTQISNCDAQMTGNEMKSWGHYAIDQHINDTCSMVAAAAAVAAGGGGGTVGCNDLQSLSLSMNPGTQSSCVTPRQISPTGLECVAIESKKRASAKVAQKQPVHRKSIDTFGQRTSQYRGVTRHRWTGRYEAHLWDNSCKKEGQTRKGRQVYLGGYDMEDKAARAYDQAALKYWGPSTHINFPLENYQKELEEMKNMTRQEYVAHLRRKSSGFSRGASIYRGVTRHHQHGRWQARIGRVAGNKDLYLGTFSTQEEAAEAYDVAAIKFRGVNAVTNFDISRYDVEKIMASNNLPAGELARRTKEREPRESIEYNNISVHKNEECVQNNNNNGNITDWKMVLYQASNPSIGSNNYRNPSFSVALQDLIGIDSINNSTSHATILDHEQNKIGANHFSNASSLVTSLGSSREASPDKTAASLVFAKPTKFVVPTTNVNACIPSAQLRPIPVSMAHLPVFAALNDA

SEQ ID NO: 10 - protein, 689 aa, Medicago truncatula
MASMNLLGFSLSPQEQHPSTQDQTVASRFGFNPNEISGSDVQGDHCYDLSSHTTPHHSLNLSHPFSIYEAFHTNNNIHTTQDWKENYNNQNLLLGTSCMNQNVNNNNQQAQPKLENFLGGHSFTDHQEYGGSNSYSSLHLPPHQPEASCGGGDGSTSNNNSIGLSMIKTWLRNQPPPPENNNNNNNESGARVQTLSLSMSTGSQSSSSVPLLNANVMSGEISSSENKQPPTTAVVLDSNQTSVVESAVPRKSVDTFGQRTSIYRGVTRHRWTGRYEAHLWDNSCRREGQTRKGRQVYLGGYDKEEKAARAYDLAALKYWGTTTTTNFPISHYEKEVEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKTILESSTLPIGGAAKRLKDMEQVELNHVNVDISHRTEQDHSIINNTSHLTEQAIYAATNASNWHALSFQHQQPHHHYNANNMQLQNYPYGTQTQKLWCKQEQDSDDHSTYTTATDIHQLQLGNNNNNTHNFFGLQNIMSMDSASMDNSSGSNSVVYGGGDHGGYGGNGGYMIPMAIANDGNQNPRSNNNFGESEIKGFGYENVFGTTTDPYHAQAARNLYYQPQQLSVDQGSNWVPTAIPTLAPRTTNVSLCPPFTLLHE

SEQ ID NO: 11 - protein, 545 aa, Cenchrus ciliaris
MGSTNNWLRFVSFSGGGGAKDAAALLPLPPSPRGDVDEAGAEPKLEDFLGLQEPSAAAVGAGRPFAVGGGASSIGLSMIKNWLRSQPAPAGPAAGVDSMVLAAAAASTEVAGDGAEGGGAVADAVQQRKAAAVDTFGQRTSIYRGVTKHRWTGRYEAHLWDNSCRREGQTRKGRQVYLGGYDKEEKAARAYDLAALKYRGTTTTTNFPMSNYEKELEEMKHMSRQEYVASLRRKSSGFSRGASIYRGVTRHHQHGRWQARIGSVAGNKDLYLGTFSTQEEAAEAYDIAAIKFRGLNAVTNFDMSRYDVKSIIESSSLPVGGAPKRLKEVPDQSDMGININGDSAGHMTAINLLTDGNDSYGAESYGYSGWCPTAMTPIPFQFSIGHDHSRLWCKPEQDNAVVAALHNLHHLQHLPAPVGTHNFFQPSPVQDMTGVADASSPPVESNSFLYNGDVGYHGAMGGSYAMPVATLVEGNSAGSGYGVEEGTGSEIFGGRNLYSLSQGSSGANTGKADAYESWDPSMLVISQKSANVTVCHGAPVFSVWK

SEQ ID NO: 12 - protein, 542 aa, unidentified
MGSTNNWLRFASFSGGGGAKDAAALLPLPPSPRGDVDEAGAEPKLEDFLGLQEPSAAAVGAGRPFAVGGGASSIGLSMIRNWLRSQPAPAGPAAGVDSMVLAAAAASTEVAGDGAEGGGAVADAVQQRKAAAVDTFGQRTSIYRGVTKHRWTGRYEAHLWDNSCRREGQTRKGRQGGYDKEEKAARAYDLAALKYRGTTTTTNFPMSNYEKELEEMKHMSRQEYVASLRRKSSGFSRGASIYRGVTRHHQHGRWQARIGSVAGNKDLYLGTFSTQEEAAEAYDIAAIKFRGLNAVTNFDMSRYDVKSIIESSSLPVGGTPKRLKEVPDQSDMGININGDSAGHMTAINLLTDGNDSYGAESYGYSGWCPTAMTPIPFQFSNGHDHSRLWCKPEQDNAVVAALHNLHHLQHLPAPVGTHNFFQPSPVQDMTGVADASSPPVESNSFLYNGDVGYHGAMGGSYAMPVATLVEGNSAGSGYGVEEGTGSEIFGGRNLYSLSQGSSGANTGKADAYESWDPSMLVISQKSANVTVCHGAPVFSVWK

SEQ ID NO: 13 - protein, 545 aa, unidentified
MGSTNNWLRFASFSGGGGAKDAAALLPLPPSPRGDVDEAGAEPKLEDFLGLQEPSAAAVGAGRPFAVGGGASSIGLSMIRNWLRSQPAPAGPAAGVDSMVLAAAAASTEVAGDGAEGGGAVADAVQQRKAAAVDTFGQRTSIYRGVTKHRWTGRYEAHLWDNSCRREGQTRKGRQVYLGGYDKEEKAARAYDLAALKYRGTTTTTNFPMSNYEKELEEMKHMSRQEYVASLRRKSSGFSRGASIYRGVTRHHQHGRWQARIGSVAGNKDLYLGTFSTQEEAAEAYDIAAIKFRGLNAVTNFDMSRYDVKSIIESSSLPVGGTPKRLKEVPDQSDMGININGDSAGHMTAINLLTDGNDSYGAESYGYSGWCPTAMTPIPFQFSNGHDHSRLWCKPEQDNAVVAALHNLHHLQHLPAPVGTHNFFQPSPVQDMTGVADASSPPVESNSFLYNGDVGYHGAMGGSYAMPVATLVEGNSAGSGYGVEEGTGSEIFGGRNLYSLSQGSSGANTGKADAYESWDPSMLVISQKSANVTVCHGAPVFSVWK

SEQ ID NO: 14 - protein, 832 aa, Rosa canina
MNMNWLGFSLSPQEDDHAPISHQLADQETLASRLGFNSNEIQGAGGGTDVSGGGSSECFDVTNSDSTASVNHHLTPSIFGIHEAAAFNRNNDHIHSQDWNMKGAGMNSSDSNNYKRTSSSDLSTMLMGSNTSTTSTSYSIISQQANLENHHQQLPKLENFLGRHSFADHDSSSAGHDYMFDMNNGPGPVSSNVMNTKTNSNNIGLSMIKTWLRNQPSQPRDNHHQLEQESKNSKESRNQPQSSLSLSMGTGSLQTVTTATAGGAATGETTSSSDKNTKQSPVVTATTTTGTDAQTQSTGGAIEAVPRKAIDTFGQRTSIYRGVTRHRWTGRYEAHLWDNSCRREGQTRKGRQVYLGGYDKEDKAARAYDLAALKYWGTTTTTNFPISSYEKEIDEMKPMTRQEYVASLRRKSSGFSRGASIYRGVTRHHQHGRWQARIGRVAGNKDLYLGTFSTQEEAAEAYDIAAIKFRGLNAVTNFDMSRYDVKSILESSALPITTGATAKRLKDVQQQQPPPPADHHHQIMLSSVLDHHGQVIRSSSSTEHDIMSNVYSAYGSYGAQQGCSWPTLAFNQAQAQAAAAPHQAPFAAGINGMQLHYSPYGYGYGNAHAQRVWCKQEQDTNSNQERSFHHQDDDHLRQQLQLGGTHNFFHDHDQQQQQQQQQTSGLMGLMDSSAASMEHSSGSNSVIYSGGDHHGNNNGYGSSTTGTGGGYIMPMVMSTVVANDDQNQADGNNNNINGFGDGDDQEANIKAQQLGYDHHPQNMFLGSSSTIDPAYQHHASNRNLYYHLPVQDDQHESSSVAVATSSTTCNMNWVPTAVPTLAHPTFTVWNDT

SEQ ID NO: 15 - protein, 828 aa, Rosa canina
MNMNWLGFSLSPQEDDHAPISHQLADQETLASRLGFNSNEIHGAGGGTDVSGGGSSECFDLTNSDSTASVNHHLTPSIFGIHEAAAFNRNNDHIHSQDWNMKGAGMNSSDSNNYKRTSSSDLSTMLMGSNTTTSTSCSIISQQANLENHHQQLPKLENFLGRHSFADHDRSSAGHDYMFDMNNGPGPVSSNVMNTKTNSNNIGLSMIKTWLRNQPSQPRDNHHQLEQESKNSKESRNQPQSSLSLSMGTGSLQTVTTATAGGAATAATGETTCSSDKNTKQSPVVTATTTTGTDAQTQSTGGAIEAVPRKAIDTFGQRTSIYRGVTRHRWTGRYEAHLWDNSCRREGQTRKGRQVYLGGYDKEDKAARAYDLAALKYWGTTTTTNFPISSYEKEIDEMKPMTRQEYVASLRRKSSGFSRGASIYRGVTRHHQHGRWQARIGRVAGNKDLYLGTFSTQEEAAEAYDIAAIKFRGLNAVTNFDMSRYDVKSILESSALPITTGATAKRLKEVQQQQPPPPADHHHQIMLSSVLDHHGQIIRSSSSTEHDIMSNVYSAYGSYGAQQGCSWPTLAFNQAQAQAAAAPHQAPFAAGINGMQLHYSPYGYGYGNAHAQRVWCKQEQDTNSNQESSFHHQDDDHLRQQLQLGGTHNFFHDHDQQQTSGLMGLMDSSAASMEHSSGSNSVIYSGGDHHGNNNGYGSSTTGTGGGYIMPMVMSTVVANDDQNQADGNNNNINGFEDGDDQEANIKAQQLGYDHHHQNMFLGSSSTIDPAYQHHASNRNLYYHLPVQDDQHESSSVAVATSSTTCNMNWVPTAVPTLAHPTFTVWNDT

SEQ ID NO: 16 - protein, 549 aa, Ricinus communis
  VARIANT 302: note = Any amino acid
MAPATTNWLSFSLSPMEMLRSSTESQFISYEGSSTATPSPHYFIDNFYANGWGNPKEAQGATTMAAETSILTSFIDPETHHQQVPKLEDFLGDSSSIVRYSDNSQTDTQDSSLTHIYDQGSAAYFSEQQDLKAIAGFQAFSTNSGSEVDDSASIARTHLGGEFMGHSIDSSGNDQLGGFSNCTAANNALSLAVNNNNNNNGNQSATNSKTIAPVIESDCPKKIADTFGQRTSIYRGVTRHRWTGRYEAHLWDNSCRREGQARKGRQGALFFLFSPSSSYHLSLFVACFFNYSSVKILGIYAXSAIPHGHVLYFFQVTNYTKELDEMKYVSKQEFIASLRRKSSGFSRGASIYRGVTRHHQQGRWQARIGRVAGNKDLYLGTFATEEEAAEAYDIAAIKFRGMNAVTNFEMSRYDVEAIMKSALPIGGAAKRLKLSLESEQKPNLNHEQQPQGSSSNSSSNNISFASMPPVTAIPCGIPFENTTAQLYHHHHHHHHHQHHNLFHHLQTTNNNLGGTTDISSGSTTSSMATTMSMLPQTAEFFLWPHHQSY

SEQ ID NO: 17 - protein, 478 aa, Elaeis guineensis
MDMDTSHSWLAFSLSYHQPYLLEALSSAPPHGGGGMTAEERGGSAEVAAMAVVGPKLEDFLGGCGEPMGRYAGGETGDAGGIYDSELKHIAAGYLQGLPATEQQDSEMAKVAAPAESRKAVETFGQRTSIYRGVTRHRWTGRYEAHLWDNSCRREGQSRKGRQVYLGGYDKEEKAARAYDLAALKYWGPTTTTNFPISNYEKELEEMKNMTRQEFVASLRRKSSGFSRGASIYRGVTRHHQHGRWQARIGRVAGNKDLYLGTFSTQEEAAEAYDIAAIKFRGLNAVTNFDISRYDVKSIANSNLPIGGMTGRPSKATESSPSSSSDAMTVEAKQLLDGRDPSASLGFAALPIKHDQDFWSLFALQQQQQQQQQQSNQASGFGLFSSGVTMDFSTASNGVISQGCGGSLVWNGGVVGQQQEQSQNNSCSSIPYATPIAFGGNYEGSSYVGSWVTPPPSYYHEPAKPNVAVFQTPIFGME

SEQ ID NO: 18 - protein, 719 aa, Populus trichocarpa
MASMNNWLGFSLSHQELPSSQSDHHQDHSQNTDSRLGFHSDEISGTNVSGECFDLTSDSTAPSLNLPATFGILEAFRNNQPQDWNMKSLGMNPDTNYKTASGLPIFMGTSCNSQTIDQNQEPKLENFLGGHSFGNHEHKLNGCNTMYDTTGDYVFQNCSLQLPSEATSNERTSNNGGGDNKNSSIGLSMIKTWLRNQPAPTQQDTNNKNNGGAQSLSLSMSTGSQSAASALPLLAVNGGVNNTGGDQSSSDNNKQQKSTTPSLDSQTGAIESVPRKSIDTFGQRTSIYRGVTRHRWTGRYEAHLWDNSCRREGQTRKGRQVYLGGYDKEEKAARAYDLAALKYWGTTTTTNFPITNYEKEIEEMKHMTRQEYVASLRRKSSGFSRGASIYRGVTRHHQHGRWQARIGRVAGNKDLYLGTFSTQEEAAEAYDIAAIKFRGLNAVTNFDMSRYDVNSILESSTLPIGGAAKRLKEAEHAEIAMDIAQRTDDHDNMGSQLTDGISSYGAVQHGWPTVAFQQAQPFSMHYPYGQRLWCKQEQDSDNRSFQELHQLQLGNTHNFFQPSVLHNLVSMDSSSMEHSSGSNSVVYSSGVNDGTSTGTNGGYQGIGYGSSAGYAVPMATVISNNDNNHNQGNGYGDGDQVKALGYENMFSPSDPYHARNLHYLSQQPSAGGIKASAYDQGSACYNWVPTAVPTIAAARSNNMAVCHGAQPFTVWNDGT

SEQ ID NO: 19 - protein, 718 aa, Populus trichocarpa
MASTNNWLGFSLSPQELPSSQSDHHDHPQNTDSRLRFHSDEISGTDVSGESFDLTSDSTAPSLNLPASFGILEAFRNNQSQDWNNMKRSGINEDTSYNTTSDVPIFMGSSCNSQNIDQNQEPKLENFLGGHSFGNHEHKLNVCSTMYGSTGHYMFHNCSLQLPSEDASNERTSSNGGADTSINNNNTNSSIGLSMIKTWLKNQPAPTQQDTNNKSNGGAQSLSLSMSTGSQSGSDLPLLAVNGGGNRTRGEQSSSDNNKQQKTTPSLDSQTGAIEVVPRKSIDTFGQRTSIYRGVTRHRWTGRYEAHLWDNSCRREGQTRKGRQGGYDKEDKAARAYDLAALKYWGTTTTTNFPMSNYEKEIEEMKHMTRQEHVASLRRKSSGFSRGASIYRGVTRHHQHGRWQARIGRVAGNKDLYLGTFSTQEEAAEAYDIAAIKFRGLNAVTNFDMNRYDVNSIMESSTLPIGGAAKRLKEAEHAEITTRVQRTDDHDSTSSQLTDGISNYGTAAHHGWPTIAFQQAQAFTMHYPYGQRLWCKQEQDSDNHSFQELHQLQLGNTQNFLQPSVLHNVMSMESSSMEHSSGSDSVMYSSGGHDGTGTGTNGSYQGIGYGSNTGYAIPMATVIANDVNTQDQGNGYGDGEVKALGYENMFSSSDPYHARNLYYLSQQSSAGVIKASAYDQGSTCNNWLPTAVPTIAARSNNMAVCHGAPTFTVWNEST

SEQ ID NO: 20 - protein, 789 aa, Larix gmelinii
MGSTSNWLAFSLSPHLTVDMPDSTQPRSTSAASNHSRHHNDFSNGTVHDCYELHPTDTMQMPLRPDGSLCILEALDRTQNNQDWQLKSLENPGSMDLESDVQSQQMMKSELSILAGGSSEQMSASIGRHKNVDQEGPKLEDFLGGASLRGHYNDARTDSIYGNDDAFDEKMMAPGLRDVVPNCLNGFDVTDTELSSGSKKTDQNQDSTRNINSIQNSLVQDSYDQNSNDQYMFQDCSLQLPPNSGANNMIGLSMIKTWLRSQPCPENKMNAATNSSTPTSAKDQSLGNLTNIQSLSLSMSPGSQSSSPLALPVQYQNTNADSPSSESKKRSLEKQSLVSVEATPRKSIDTFGQRTSIYRGVTRHRWTGRYEAHLWDNSCRREGQTRKGRQVYLGGYDKEEKAARAYDLAALKYWGPTTTTNFPTGNYEKELEEMKHMTRQEYVASLRRKSSGFSRGASIYRGVTRHHQHGRWQARIGRVAGNKDLYLGTFSSQEEAAEAYDIAAIKFRGLNAVTNFDMTRYDVNSILESSTLPIGGAAAKRIKDAEPSDPSVDGRRTDDEISSTISSQIADTLTSYGNAAYPNGHAGWPIIAFQQQTNPHAPAFYSQQRAAAGWCKQEHNNIQNHDLQLHFQSSTQNFLQPSMMTSNANTVLHNLMNLESSAQLDGTNTNSNSGLFSNISGNLAGNSLQMANSPIPSGITVCDSARTPFSTENDGSSTKNSSYNDNMLSNSDPFARGLYYLSQHSPSVVKANYENAAYNNWMTPAVQTLAPRPNLTVCHAPIFTVWNDT

SEQ ID NO: 21 - DNA, 1361 bp, Arabidopsis sp.
  source 1..1361: mol_type = genomic DNA
cctctttgtcaccgtcactcttctcctcgttctcaacgtctccagcagagcactcccgcccgtggcggattccaccaacatagcggctagactaaccggaggaggactgatgcagtgttgggatgcactctacgagctgaagtcatgtactaatgagatcgttctcttctttctcaacggtgagaccaaactcggctacggttgctgcaacgccgttgatgtcattaccactgattgttggccggcgatgcttacttctcttggctttacactggaggaaaccaatgtcctccgtggtttctgtcaatctccgaactccggcggttcttctccagctctttcccctgtcaaactttgataaatgttcctcgctgacgtaagaagacattagtaatggttataatatatagctttctatgaatgtatggtgagaaaatgtctgttcactgattttgagtttggaataaaagcatttgcgtttggtttatcattgcgtttatacaaggacagagatccactgagctggaatagcttaaaaccattatcagaacaaaataaaccattttttgttaagaatcagagcatagtaaacaacagaaacaacctaagagaggtaacttgtccaagaagatagctaattatatctattttataaaagttatcatagtttgtaagtcacaaaagatgcaaataacagagaaactaggagacttgagaatatacattcttgtatatttgtattcgagattgtgaaaatttgaccataagtttaaattcttaaaaagatatatctgatctagatgatggttatagactgtaattttaccacatgtttaatgatggatagtgacacacatgacacatcgacaacactatagcatcttatttagattacaacatgaaatttttctgtaatacatgtctttgtacataatttaaaagtaattcctaagaaatatatttatacaaggagtttaaagaaaacatagcataaagttcaatgagtagtaaaaaccatatacagtatatagcataaagttcaatgagtttattacaaaagcattggttcactttctgtaacacgacgttaaaccttcgtctccaataggagcgctactgattcaacatgccaatatatactaaatacgtttctacagtcaaatgctttaacgtttcatgattaagtgactatttaccgtcaatcctttcccattcctcccactaatccaactttttaattactcttaaatcaccactaagcttcgaatccatccaaaaccacaatataaaaacagaactctcgtaactcaatcatcgcaaaacaaaacaaaacaaaacaaaaaccccaaaaagaaagaata

SEQ ID NO: 22 - DNA, 678 bp, Oryza sativa
  source 1..678: mol_type = genomic DNA
atggaatgatggatgaatgttcacgttcttgagttcctaaatggtactaattttgcaaaaactttctatatgtgttttttgttaagaatgttgttttaaacccatcttttcactttataatatttaattaaatcgttcgtaccctcgaatagttattgcaaattatacttaactattcagtcattcagcacaaaagaacagggccatgaaattgtaatactagtacatttctgttcttttcttttctttttgaggttgtctgaaacacctgtatcttaaactatcgcagactagccaatgagtcgtactcacctgaaactgaaaccaagtgattaaccaagctggttcgacagtaattccatccataatgcagctccggagcccttcatatcctgcatgttactcaaacaacatccccacctcctcatttcctctcccctattgcattgcataattgcagaagattaagccgctaatgcataattacacattatttgtgtccactaattttccctttcccacacgctacgaaactcaaaagccggcctcctcgcctccttccctgaacgttactaatcgcgtcatgtataaatacagagcttgcccacgcaccggcacattgcatcgcactacgcacatctacacgatacccaagcagcaaagctagaaagaaaaacc

SEQ ID NO: 23 - DNA, 465 bp, Arabidopsis thaliana
  source 1..465: mol_type = genomic DNA
gttgccttatgatttcttcggtttcaagatgatcaaatagttatagatttcatgctcacacatgctcattagatgtgtacatactttacttacccaaatctattttctcgcaaagattttgatggtaaagctgatttggttctattgaactaaatcaaacgagtttcagactgagtgattctaatccggcccattagcccctaaacagacccactaattacgcagcttttaatagagtaattacacctagtttacccactaaaccactaagcactaattatctcacaatctaatgagcttccctcgtaattacttgggctttcactctaccatttatttgtaacagtcaagtctctactgtctctatataaactctctaaagttaacacacaattctcatcacaaacaaatcaaccaaagcaacttctactctttcttctttcgaccttatcaatctgttgagaa

SEQ ID NO: 24 - DNA, 705 bp, Oryza sativa
  source 1..705: mol_type = genomic DNA
atgcctgaagtgagaaattccggcggtagggcggcgctcgccgacccctcgggtggtgggttctttatcaggaggacgacgtcgccgccgggagccgtggcggtcaagccgctggctcggcgggccctgccgccgacgagcaacaaggagaacgtgccgccgtcctgggctgtgaccgtgagggctacacccaagaggaggagccccctgcccgagtggtacccgaggagcccactccgcgacatcacgtcagtcgtcaaggcagttgagaggaaaagtcgcctcggaaatgctgcggttcggcagcagatccagttgagtgaagattcttcacgatctgtggatccagcaactccagtacaaaaagaagaaggtgtccctcaaagcacaccaacaccaccaactcaaaaggccctggatgctgctgccccttgtcctggctcaacccaagctgttgcaagcacatcaacagcttacttggccgagggcaagccgaaggcatcatcttcttctccatctgactgctcctttcagacaccatccagaccaaatgatccagctcttgctgatctcatggagaaggaactgtccagctccatagagcagatagagaagatggtaaggaagaacctcaagagagctccgaaggctgctcagccttccaaggtgaccatccagaagcgcaccctgttgtccatgagatga

SEQ ID NO: 25 - protein, 234 aa, Oryza sativa
MPEVRNSGGRAALADPSGGGFFIRRTTSPPGAVAVKPLARRALPPTSNKENVPPSWAVTVRATPKRRSPLPEWYPRSPLRDITSVVKAVERKSRLGNAAVRQQIQLSEDSSRSVDPATPVQKEEGVPQSTPTPPTQKALDAAAPCPGSTQAVASTSTAYLAEGKPKASSSSPSDCSFQTPSRPNDPALADLMEKELSSSIEQIEKMVRKNLKRAPKAAQPSKVTIQKRTLLSMR

SEQ ID NO: 26 - DNA, 732 bp, Arabidopsis thaliana
  source 1..732: mol_type = genomic DNA
atgccagaagcaagagatcgaaccgagaggcctgtggattactcgactatatttgccaaccgacggagacatggtattttacttgacgagccagattcacggcttagtttgattgaatctccggtgaatccagatattgggtctattggtggaacgggcgggcttgtgagaggcaatttcactacatggaggcctggtaatggcagaggtggtcacactccatttagattgccacagggaagagagaatatgcccatagtgaccgctaggcgtggaagaggtggtggtttgttgccttcttggtatccaagaacacctctacgcgacataactcatattgtgcgggctattgagagaagaagaggagctgggactggaggagacgatggccgagttattgagatcccaactcatcgacaagttggtgttcttgaatctccagtaccactgtcaggagaacacaaatgctcgatggtcactcctggaccatctgtgggattcaagcgtagttgcccaccatcaactgctaaagttcaaaagatgttacttgacatcactaaagagatagctgaggaagaagctggcttcatcacacccgagaagaagctactcaattctattgacaaagttgagaaaattgtgatggcggagatccagaagttgaagagcactcctcaagctaaaagggaagagcgggagaagagggtgcggactttaatgactatgcgatga

SEQ ID NO: 27 - protein, 243 aa, Arabidopsis thaliana
MPEARDRTERPVDYSTIFANRRRHGILLDEPDSRLSLIESPVNPDIGSIGGTGGLVRGNFTTWRPGNGRGGHTPFRLPQGRENMPIVTARRGRGGGLLPSWYPRTPLRDITHIVRAIERRRGAGTGGDDGRVIEIPTHRQVGVLESPVPLSGEHKCSMVTPGPSVGFKRSCPPSTAKVQKMLLDITKEIAEEEAGFITPEKKLLNSIDKVEKIVMAEIQKLKSTPQAKREEREKRVRTLMTMR

SEQ ID NO: 28 - DNA, 1479 bp, Oryza sativa
  source 1..1479: mol_type = genomic DNA
atgaagcttaagatgaacaaggcctgcgacatcgcctccatctccgtcctccctccccggaggaccggagggagcagcggcgcgtcggcttccggttccgtggcggtggcggtggcgtctcagccgcggtcgcagccgctctcgcagtcgcagcagtccttctcgcagggcgcctccgcctcgctcttgcactcgcagtcgcagttctcgcaggtctccctcgacgacaacctcctcaccctcctcccttcccccacccgcgatcagagatttggcttgcatgatgactcatccaagaggatgtcctctttaccagccagttcagcttcttgcgcgcgagaagagtctcagctgcaactggcaaaattaccaagcaacccagtgcaccgctggaacccctccattgcagatactagatcaggtcaggttactaatgaggatgttgagcgcaaatttcagcatctggcaagctcagtacataagatggggatggtggtagactcagtccaaagtgacgtaatgcagttaaacagagccatgaaggaggcatcattagattctggtagcatacggcaaaagattgctgtccttgaaagctcacttcagcaaattcttaagggacaagacgatctcaaagcactctttggaagcagcacaaaacacaatcctgatcagacaagtgttctgaattctctaggcagcaaattgaatgagatatcctcgacccttgcaaccttgcagacacaaatgcaagcaagacaactgcagggtgatcagacaactgttctgaattctaatgccagcaaatcgaatgagatatcctcgactcttgcaaccctgcagacacaaatgcaagcagatataagacaactgcggtgtgacgtcttcagagtttttacaaaagagatggagggggttgttagagctatcaggtctgtcaatagtaggcctgctgcaatgcaaatgatggcagaccagagttaccaagtaccagtttcaaatggatggacccagattaaccagacaccagtagcagctggaaggtctccaatgaaccgagcaccagtagcagctggaaggtcccggatgaaccaattacctgaaacaaaagtgctttctgcacatttggtttatcctgcaaaggtgacagatctgaagccaaaggtggagcagggaaaggtaaaagcagctccacaaaagccgtttgcttcgagctactacagggtggcacctaaacaggaagaggtagcgattagaaaggtcaatatacaagtgccagcaaagaaggcaccagtcagcataatcatcgagtcggatgatgacagtgaaggacgtgcgtcctgcgtgattttgaagacagaaacaggtagcaaggagtggaaagtgacaaagcaaggcaccgaagagggcctggagatcctgcggagggcgaggaagaggaggaggagagagatgcagtccatcgtgctcgcatcctag

SEQ ID NO: 29 - protein, 492 aa, Oryza sativa
MKLKMNKACDIASISVLPPRRTGGSSGASASGSVAVAVASQPRSQPLSQSQQSFSQGASASLLHSQSQFSQVSLDDNLLTLLPSPTRDQRFGLHDDSSKRMSSLPASSASCAREESQLQLAKLPSNPVHRWNPSIADTRSGQVTNEDVERKFQHLASSVHKMGMVVDSVQSDVMQLNRAMKEASLDSGSIRQKIAVLESSLQQILKGQDDLKALFGSSTKHNPDQTSVLNSLGSKLNEISSTLATLQTQMQARQLQGDQTTVLNSNASKSNEISSTLATLQTQMQADIRQLRCDVFRVFTKEMEGVVRAIRSVNSRPAAMQMMADQSYQVPVSNGWTQINQTPVAAGRSPMNRAPVAAGRSRMNQLPETKVLSAHLVYPAKVTDLKPKVEQGKVKAAPQKPFASSYYRVAPKQEEVAIRKVNIQVPAKKAPVSIIIESDDDSEGRASCVILKTETGSKEWKVTKQGTEEGLEILRRARKRRRREMQSIVLAS

SEQ ID NO: 30 - DNA, 1152 bp, Arabidopsis thaliana
  source 1..1152: mol_type = genomic DNA
atggaggaaagttcaggactatcatcgatgaagttcttctccgatcaacacctctcttacgctgacattcttcttcctcacgaagctcgtgctagaatcgaagtttctgttctcaatctcctccgaattttgaactccccagatccagctatctccgatctctctctgatcaataggaagagaagcaatagttgtataaacaaagggatactcacagatgtttcatatatattcctctctacttcgtttactaagagttcattgacgaatgctaaaacagctaaagcttttgttcgtgtgtggaaagttatggaaatatgctttcagattctgcttcaagagaaacgagtcacacaaagagagctcttctataagttgctttgtgattcacctgattacttttcatctcagattgaagttaacagaagtgtccaagatgtggtagcacttctacgttgtagtagatacagtcttggtattatggcttcaagcagaggccttgttgctggaaggttatttctacaagaaccaggtaaggaagctgtagattgttcggcctgtggttcttcaggttttgctataactggagacttgaatttgctagacaataccatcatgagaactgatgctcgttatatcattattgtggaaaagcatgcgatctttcatcggctcgtggaagatcgtgtgttcaatcacattccttgcgtgttcatcacagcgaaagggtatccggatattgccacaaggtttttcctccaccggatgagcacaacttttcctgatctgccaattcttgttctagttgattggaatccagctgggttagctatactatgcaccttcaagtttggaagcatagggatgggacttgaagcatacagatacgcttgcaatgtgaagtggattggtctccgaggagatgatctgaatctgataccagaagagtctttggttcccttaaagccaaaagattcacagattgctaagagcttattgtcctccaaaatattgcaggaaaactacatagaggagttgtcactgatggttcaaactggtaaaagagcggaaattgaagctctctattgtcatggttataattatctcggtaaatatatagctaccaagatcgtgcaaggcaaatacatataa

SEQ ID NO: 31 - protein, 383 aa, Arabidopsis thaliana
MEESSGLSSMKFFSDQHLSYADILLPHEARARIEVSVLNLLRILNSPDPAISDLSLINRKRSNSCINKGILTDVSYIFLSTSFTKSSLTNAKTAKAFVRVWKVMEICFQILLQEKRVTQRELFYKLLCDSPDYFSSQIEVNRSVQDVVALLRCSRYSLGIMASSRGLVAGRLFLQEPGKEAVDCSACGSSGFAITGDLNLLDNTIMRTDARYIIIVEKHAIFHRLVEDRVFNHIPCVFITAKGYPDIATRFFLHRMSTTFPDLPILVLVDWNPAGLAILCTFKFGSIGMGLEAYRYACNVKWIGLRGDDLNLIPEESLVPLKPKDSQIAKSLLSSKILQENYIEELSLMVQTGKRAEIEALYCHGYNYLGKYIATKIVQGKYI

SEQ ID NO: 32 - DNA, 1827 bp, Oryza sativa
  source 1..1827: mol_type = genomic DNA
atgttctactcgcaccagctcctcgcgcggaaggctccgctcggccagatatggatggcggcgacgcttcactcgaagatcaaccggaagcggcttgacaagctcgacatcatcaaaatctgtgaggagattttgaacccgtcggtacccatggcactaaggctctccggaattctcatgggtggtgtggcgatcgtgtacgagaggaaggtgaaggctctgtatgatgatgtgtctcggtttctgattgagatcaacgaggcatggcgggtcaagccagtcgcagaccccaccgtacttcccaagggcaaaacccaagccaagtatgaagcagtaacactgccagagaatatcatggatatggatgtggagcagcccatgcttttctcagaggctgatactacaaggttccggggaatgcgtttggaggatttggatgaccaatacattaatgtcaacctagacgatgatgacttctcgcgcgctgagaatcatcaccaagctgatgcagaaaatatcaccctggctgataatttcgggtctgggcttggagagactgatgtgttcaatcgttttgagagattcgacataacagatgatgatgcaactttcaatgtcactcctgatggacacccacaggttccaagtaatctggttccttctccacctaggcaggaagactctcctcagcaacaagaaaaccatcatgctgcctcatcccctcttcacgaagaagctcaacaagggggggcatctgtaaaaaatgagcaagagcagcagaagatgaagggtcagcaacctgctaaatcatcaaagagaaaaaaacgtaggaaagatgatgaggtgatgatggataacgaccagataatgatcccaggaaatgtatatcaaacatggctgaaggatccatcaagcctcattaccaaaaggcacagaatcaacagtaaagttaatcttattcggtcaatcaagataagagacctcatggacttgcccctcgtttctctaatatcttccttggagaagtcacccttagaattttattatcctaaggaacttatgcagctttggaaggaatgtactgaagtcaagtccccaaaagctccatcttcaggagggcagcagtcatcatcaccagaacaacagcaaagaaacttgcctcctcaggcatttccaacccagcctcaggttgataatgacagggaaatgggatttcacccagtggactttgcagatgacatcgaaaaactccgaggaaacactagtggggaatatggaagagattatgatgcttttcacagtgatcatagtgttactcctggaagtcctgggctaagtcgcaggtctgcttcaagctctggtggctctggacggggatttacgcagttggatccagaagtacagttgccatccggaaggtccaagaggcagcattcatctggaaaaagctttgggaacctcgatccagttgaagaagaattcccattcgagcaagaacttagagatttcaagatgagaaggctttcagatgttgggccaactccagacctgctggaagaaatcgaacctactcaaaccccatatgaaaagaaatccaatcctatcgaccaggtcacacaatcaatccactcgtacctcaagctacactttgacaccccaggggcctcacagtctgaatcattaagtcagctagcacatgggatgactacagcaaaggctgcccgactcttctatcaagcatgcgttttagcaactcatgattttatcaaggttaaccagctggaaccatacggagacatcttgatctcgaggggaccaaagatgtga

SEQ ID NO: 33 - protein, 608 aa, Oryza sativa
MFYSHQLLARKAPLGQIWMAATLHSKINRKRLDKLDIIKICEEILNPSVPMALRLSGILMGGVAIVYERKVKALYDDVSRFLIEINEAWRVKPVADPTVLPKGKTQAKYEAVTLPENIMDMDVEQPMLFSEADTTRFRGMRLEDLDDQYINVNLDDDDFSRAENHHQADAENITLADNFGSGLGETDVFNRFERFDITDDDATFNVTPDGHPQVPSNLVPSPPRQEDSPQQQENHHAASSPLHEEAQQGGASVKNEQEQQKMKGQQPAKSSKRKKRRKDDEVMMDNDQIMIPGNVYQTWLKDPSSLITKRHRINSKVNLIRSIKIRDLMDLPLVSLISSLEKSPLEFYYPKELMQLWKECTEVKSPKAPSSGGQQSSSPEQQQRNLPPQAFPTQPQVDNDREMGFHPVDFADDIEKLRGNTSGEYGRDYDAFHSDHSVTPGSPGLSRRSASSSGGSGRGFTQLDPEVQLPSGRSKRQHSSGKSFGNLDPVEEEFPFEQELRDFKMRRLSDVGPTPDLLEEIEPTQTPYEKKSNPIDQVTQSIHSYLKLHFDTPGASQSESLSQLAHGMTTAKAARLFYQACVLATHDFIKVNQLEPYGDILISRGPKM

SEQ ID NO: 34 - DNA, 1884 bp, Arabidopsis thaliana
  source 1..1884: mol_type = genomic DNA
atgttgagactggagagtttgatagtaacagtgtggggaccagcgacgcttctagctcgaaaagctccgttgggtcagatatggatggccgctacattgcacgcgaagatcaaccggaagaaactagataagctcgatatcattcaaatctgcgaagagattttgaatccgtcggttccgatggctcttagactctccgggattcttatgggtggtgttgtgattgtttatgagaggaaagtgaagctcctattcgatgatgttaatcgctttctggttgaaattaatggagcttggcgcacaaaatctgttccggatcccactttactacctaaaggaaaaacccatgccaggaaagaggctgttacattgcctgagaacgaagaagctgattttggagattttgaacagactcgtaatgttcctaaatttggcaattacatggattttcagcagacttttatttccatgcggttagatgaatcccatgttaacaataaccccgagccagaagatcttggacagcagttccatcaagctgatgccgagaatatcacactctttgagtatcatggttcattccagaccaacaatgaaacatatgatcgttttgaaagatttgacatcgaaggagatgatgaaacacagatgaactccaatccaagagaaggcgctgaaatacctacaactctcatcccatcaccacctcgtcatcatgacattcccgaaggagtcaaccccacaagccctcagcgccaggagcaacaggagaatcgtagggacggatttgctgagcagatggaggaacaaaacataccggacaaagaggaacacgatagaccacaaccagcgaaaaagagagcaagaaagacagctacttcagcgatggattatgagcaaactattatcgctggtcatgtttaccagtcatggctccaggatacttctgacattctctgtaggggggaaaagagaaaggttcgaggaactatccggccagacatggaaagtttcaaacgtgcgaatatgccacctacacaactctttgaaaaggacagttcttacccgcctcagctttaccagctttggtcaaagaatactcaagttcttcaaacctcatcatctgaatctcgacatcctgatctccgtgcggaacaatctccagggtttgttcaggagagaatgcataaccaccatcaaacagaccatcatgagcgcagtgacacaagctcccaaaatcttgatagtcccgcagaaatactccggacagttcgtactgggaaaggtgcttcagtagaaagcatgatggctggatctcgagcaagccctgaaactattaaccgccaggctgctgatattaatgtcacgccattctattctggagatgatgtgagatccatgcctagtacaccatccgcacgtggagcagcttcaattaacaacatagagatcagctctaaaagtcgcatgcccaatagaaaaagaccaaattcctcaccaagaagaggactcgaaccagtggcggaagagagaccgtgggagcaccgtgaatatgagtttgagttttcaatgttacctgaaaaacgcttcacagccgataaagaaatactatttgaaactgcatctacacagactcaaaagccagtgtgcaatcaatcagacgagatgataacagatagcatcaaaagtcacctgaagacacactttgaaacacctggagctcctcaagtggaatctcttaacaagctcgctgttggaatggacagaaacgctgcagctaaactcttcttccaatcctgtgtgttagctactcgcggagtcatcaaggtaaaccaagcagagccttatggggacattctcattgcaagaggacccaacatgtaa

SEQ ID NO: 35 - protein, 627 aa, Arabidopsis thaliana
MLRLESLIVTVWGPATLLARKAPLGQIWMAATLHAKINRKKLDKLDIIQICEEILNPSVPMALRLSGILMGGVVIVYERKVKLLFDDVNRFLVEINGAWRTKSVPDPTLLPKGKTHARKEAVTLPENEEADFGDFEQTRNVPKFGNYMDFQQTFISMRLDESHVNNNPEPEDLGQQFHQADAENITLFEYHGSFQTNNETYDRFERFDIEGDDETQMNSNPREGAEIPTTLIPSPPRHHDIPEGVNPTSPQRQEQQENRRDGFAEQMEEQNIPDKEEHDRPQPAKKRARKTATSAMDYEQTIIAGHVYQSWLQDTSDILCRGEKRKVRGTIRPDMESFKRANMPPTQLFEKDSSYPPQLYQLWSKNTQVLQTSSSESRHPDLRAEQSPGFVQERMHNHHQTDHHERSDTSSQNLDSPAEILRTVRTGKGASVESMMAGSRASPETINRQAADINVTPFYSGDDVRSMPSTPSARGAASINNIEISSKSRMPNRKRPNSSPRRGLEPVAEERPWEHREYEFEFSMLPEKRFTADKEILFETASTQTQKPVCNQSDEMITDSIKSHLKTHFETPGAPQVESLNKLAVGMDRNAAAKLFFQSCVLATRGVIKVNQAEPYGDILIARGPNM

SEQ ID NO: 36 - DNA, 1329 bp, Arabidopsis thaliana
  source 1..1329: mol_type = genomic DNA
atgtcttcttcgtcgagaaatctatctcaggagaatccgattcctcgtccgaacttagccaagactcgaacctcactccgcgatgttggaaaccgtcgtgctcccctcggcgacatcacaaatcagaagaatggatctagaaatccttcaccgtcgtctactctggtgaattgttcaaataagatcggccaatctaagaaagcaccaaaacctgctttatctcgtaattggaatttgggaattctcgattccggtttacctcccaagccaaatgcgaaatcaaacataatcgttccttacgaagacaccgaattgctccaaagcgatgatagtcttctatgttcttcacctgcattatccttggatgcctctcctactcaatctgacccgtcaatttccactcatgactctttgacgaaccacgttgtagattacatggtcgagagcactactgatgatggaaatgatgatgatgatgatgagattgttaacattgatagtgacttgatggatccacagctttgtgcttcttttgcttgtgatatctacgagcatttgcgtgtatctgaggtgaacaaaagaccggctctagattacatggaaagaactcagtcaagcatcaatgctagcatgcgttctatactgattgactggcttgtggaggttgctgaagagtataggctttcgcccgagacgttgtatttggcagtaaactacgttgatcggtatcttacaggaaatgcaatcaacaagcaaaatctgcagctacttggtgttacctgcatgatgatagcagcaaaatatgaagaagtgtgtgtgccgcaagtggaggatttctgttacatcactgataacacatacttaagaaatgagcttttggagatggagtcttctgttctgaactacttgaagttcgaattaacaactccaacagcaaaatgtttcttgaggcgctttcttcgtgctgctcaaggcagaaaggaggtaccatcactgctgtctgagtgtctggcctgctatctcaccgaattatcgctgttagattacgctatgcttcgatacgctccatcacttgttgcagcctctgcagttttcttggcacaatacactctacacccttcaagaaaaccatggaatgctacgctagagcattacacatcgtacagggctaaacatatggaagcatgcgttaagaatcttcttcagctgtgtaatgagaaactctcatctgatgtggttgcaatcagaaagaagtacagtcaacacaaatacaagtttgcagcaaagaagctttgtcccacgtcactaccgcaagagcttttcctctga

SEQ ID NO: 37 - protein, 442 aa, Arabidopsis thaliana
MSSSSRNLSQENPIPRPNLAKTRTSLRDVGNRRAPLGDITNQKNGSRNPSPSSTLVNCSNKIGQSKKAPKPALSRNWNLGILDSGLPPKPNAKSNIIVPYEDTELLQSDDSLLCSSPALSLDASPTQSDPSISTHDSLTNHVVDYMVESTTDDGNDDDDDEIVNIDSDLMDPQLCASFACDIYEHLRVSEVNKRPALDYMERTQSSINASMRSILIDWLVEVAEEYRLSPETLYLAVNYVDRYLTGNAINKQNLQLLGVTCMMIAAKYEEVCVPQVEDFCYITDNTYLRNELLEMESSVLNYLKFELTTPTAKCFLRRFLRAAQGRKEVPSLLSECLACYLTELSLLDYAMLRYAPSLVAASAVFLAQYTLHPSRKPWNATLEHYTSYRAKHMEACVKNLLQLCNEKLSSDVVAIRKKYSQHKYKFAAKKLCPTSLPQELFL

SEQ ID NO: 38 - DNA, 1038 bp, Oryza sativa
  source 1..1038: mol_type = genomic DNA
atgtcgacgtgcgactcaatgaaaagcccagactttgagtatattgataatggggattcctcctcagttctaggttccttgcagcgaagagcaaacgagaacctgcgtatctcagaggatagagatgttgaagaaactaagtggaagaaggatgctccttccccaatggaaatcgaccaaatttgtgatgttgacaataactacgaggatccgcagttgtgtgctactcttgcttctgatatctacatgcacttgcgcgaggccgagaccaggaaacatccatcaaccgattttatggaaacactccaaaaggatgtaaacccaagcatgagagcgatcctgatagactggcttgtggaagtcgctgaagaatatcgtcttgttcctgatacattatacctgacagttaactacattgaccgttatctttctggcaatgagatcaatcgtcaaagactgcaattacttggagttgcttgtatgcttattgctgcaaaatacaaggagatatgtgcacctcaagtagaagaattctgctatataactgacaacacatacttcagagatgaggttttggaaatggaagcttctgtcctgaattacctgaagtttgaaatgactgcacctacagcaaaatgctttttgaggagatttgtccgtgttgcacaagtatctgatgaggatccagcattgcatcttgagttcctagccaattatgttgctgagctatcactgctggagtacaatctactttcttaccctccttcactagtagcggcatcagctattttcctggccaaattcatactgcagccagcaaagcacccttggaattccacccttgctcactacacacaatacaagtcgtcagagttaagcgactgcgttaaggcattgcaccgccttttctgtgttggtcctgggagtaaccttcctgcaatcagggagaagtatacccaacataagtacaaatttgtggcgaagaagccctgcccaccctcaataccgaccgaattctttcgcgactcaacatgctga

SEQ ID NO: 39 - protein, 345 aa, Oryza sativa
MSTCDSMKSPDFEYIDNGDSSSVLGSLQRRANENLRISEDRDVEETKWKKDAPSPMEIDQICDVDNNYEDPQLCATLASDIYMHLREAETRKHPSTDFMETLQKDVNPSMRAILIDWLVEVAEEYRLVPDTLYLTVNYIDRYLSGNEINRQRLQLLGVACMLIAAKYKEICAPQVEEFCYITDNTYFRDEVLEMEASVLNYLKFEMTAPTAKCFLRRFVRVAQVSDEDPALHLEFLANYVAELSLLEYNLLSYPPSLVAASAIFLAKFILQPAKHPWNSTLAHYTQYKSSELSDCVKALHRLFCVGPGSNLPAIREKYTQHKYKFVAKKPCPPSIPTEFFRDSTC

SEQ ID NO: 40 - DNA, 1071 bp, Oryza sativa
  source 1..1071: mol_type = genomic DNA
atgtctaaggaagatgctatgtcaactggtgattcaacggaaagccttgatattgattgccttgatgatggggactccgaagtggtatcttccttgcaacatttggcagatgataagcttcatatttctgacaacagggatgttgcaggtgtggcatccaaatggacgaagcatggttgtaattcagtagaaattgattatatcgtcgacattgacaacaaccatgaggatccacagctgtgtgcaactcttgcttttgacatttacaagcacttgcgagtggctgagaccaagaaaaggccttcaacagattttgtggaaaccattcagaagaacattgacacaagcatgagggcagtgttaatagactggcttgtggaagtcacagaagaatatcggcttgtacctgaaaccttatacctcacagtcaattacattgaccggtatctctcgagcaaggtgatcaatcggcggaaaatgcaattacttggtgtcgcttgcctgcttatagcttctaagtatgaagagatatgcccaccccaagtagaagagctctgctatatttctgacaatacatacactaaggatgaggttttgaaaatggaagcttctgtcctgaaatacttgaagtttgagatgactgcacctacaacaaaatgctttttgaggagatttctacgagctgctcaagtatgccatgaggctccagttttgcatcttgagttcctagctaattacattgcggagctatcacttctggagtacagcttaatttgctatgtaccgtcacttatagctgcgtcttctattttcttggcgaagtttatccttaagccaacagagaatccttggaattcaacactttcattctacacacaatacaaaccatccgacctatgcaattgtgcaaaaggactacaccggcttttcttggttggccctggaggcaaccttcgagcagttagagaaaaatacagtcaacacaagtacaaattcgtagcaaagaagtactctccaccatcaattccagcagagtttttcgaagatccaagcagctacaagcctgattaa

SEQ ID NO: 41 - protein, 356 aa, Oryza sativa
MSKEDAMSTGDSTESLDIDCLDDGDSEVVSSLQHLADDKLHISDNRDVAGVASKWTKHGCNSVEIDYIVDIDNNHEDPQLCATLAFDIYKHLRVAETKKRPSTDFVETIQKNIDTSMRAVLIDWLVEVTEEYRLVPETLYLTVNYIDRYLSSKVINRRKMQLLGVACLLIASKYEEICPPQVEELCYISDNTYTKDEVLKMEASVLKYLKFEMTAPTTKCFLRRFLRAAQVCHEAPVLHLEFLANYIAELSLLEYSLICYVPSLIAASSIFLAKFILKPTENPWNSTLSFYTQYKPSDLCNCAKGLHRLFLVGPGGNLRAVREKYSQHKYKFVAKKYSPPSIPAEFFEDPSSYKPD

SEQ ID NO: 42 - DNA, 2046 bp, Oryza sativa
  source 1..2046: mol_type = genomic DNA
atggccttggtgtgtgcggaatgccgtcttgttgacatcgtccgggtgcatgcgaacctgatggtacccgaaatggagatccagcttggagaagaagtgggcgtcgcaaagttcatcaagcaattcgtcgagaaccaaaatgggaaacatgtccttggcagtcaccgtgttgagggctcggtagtccacgtagaagcgccatggcttgtggaccaacagaaccgacgacgagaatggcgacgtgttggggcagatggagagcttgtcagcgcattatttctccaattcgtcgttttgaagctgtgggtagcgatacggtcgaaccactaccgggggcttgccgggcttgaggtggatgtgatgattgatggatcgggtcggtgggaggccggttggagcgcgaagaggtcgtcaaattccgccagcaaggacggaaggagatcgtcggcgtgcagtgcagcgcagctgaggtcgagggcgaagcagtcgacgtcgaacacctcggtggccacctacaggcgaagcccgtgcacgacacccgcgccgcggatgcgatcgttgtcagccaccaccacgcggagaccttggcgtaccggttggagaggtaggccgatgcgctgggccagcttggtggtgatgaaattgtgtgttgaagccggagtcaacaagggccatcacctgctcggccgcgatacgcgccacgaggcacatcgtgctagtgccattgacaccgaaaatcgcgttgagcgagatgtgtggttcatcggtgtcgtctgtgtcgtcccattcgctgaaattagccgtggtgtcgtcgtactcaagaggccttgtttacagcgctctggtaactctgctggcgagagacaacgaaattggtgtcctggcacgagccaaggccatggcctcctggaaatcctcgggctgttggagctcgacatcgatgcagatgtcgtcggtgagccccgctgtgaagagctgaacttgttggcggtcggtgaggaggtcggacgtgcggctacccaacgccaaaaggcgctgctggtacgtcgtcaccgtgccaacctgagtgagatgcttgagttcacccagggaattgttgcggatcggcggtccatactgaccgtaacagagctgcttgaacgtatcccaatccggaggacccatgtgctgctcataatggaagtaccattcctgtgcagctcaagtgagatgaccaggaaacgtccatcaactgattttatggaaacaatccaaaaggatgtaaacccaagcatgagagcgatcctgatagactggcttgtggaagtcgctgaagaatatcgtcttgttcctgatacattatacctgacagttaactacattgatcgttatctttctggcaatgagatcaatcgtcaaagactgcaattacttggagttgcttgtatgcttattgctgcaaaatacgaggagatatgtgcacctcaagtagaagaattctgctatataactgacaacacatacttcagagatgagtgctggaatgaatcgaactctaataactctcttattgcctacaacaggagatttgtccgtgttgcacaagtatcggatgagcttttcatcgtgcaggatccagcattgcatcttgagttcctagccaattatgttgctgagctatcactgctggagtacaatctactttcttaccctccttcactagtagcggcatcggctattttcttggccaaattcatactgcagccaacaaagcacccttggaattccacccttgctcactacacacaatacaagtcgtcagagttaagcgactgtgtaaaggcattgcaccgcctttttagcgttggtcccgggagtaaccttcctgcaatcagggagaagtatacccaacataagatactgcatgcagctgatgtgatcgacttgaacatggcaaatgcatttaagaatgtgaaaatattatgtcaatgtccctgtcaatgcaaccttcttgaagaagtcatgctcaagctatttccatactggaagctaagcacagctgtttag

SEQ ID NO: 43 - protein, 681 aa, Oryza sativa
MALVCAECRLVDIVRVHANLMVPEMEIQLGEEVGVAKFIKQFVENQNGKHVLGSHRVEGSVVHVEAPWLVDQQNRRREWRRVGADGELVSALFLQFVVLKLWVAIRSNHYRGLAGLEVDVMIDGSGRWEAGWSAKRSSNSASKDGRRSSACSAAQLRSRAKQSTSNTSVATYRRSPCTTPAPRMRSLSATTTRRPWRTGWRGRPMRWASLVVMKLCVEAGVNKGHHLLGRDTRHEAHRASAIDTENRVERDVWFIGVVCVVPFAEISRGVVVLKRPCLQRSGNSAGERQRNWCPGTSQGHGLLEILGLLELDIDADVVGEPRCEELNLLAVGEEVGRAATQRQKALLVRRHRANLSEMLEFTQGIVADRRSILTVTELLERIPIRRTHVLLIMEVPFLCSSSEMTRKRPSTDFMETIQKDVNPSMRAILIDWLVEVAEEYRLVPDTLYLTVNYIDRYLSGNEINRQRLQLLGVACMLIAAKYEEICAPQVEEFCYITDNTYFRDECWNESNSNNSLIAYNRRFVRVAQVSDELFIVQDPALHLEFLANYVAELSLLEYNLLSYPPSLVAASAIFLAKFILQPTKHPWNSTLAHYTQYKSSELSDCVKALHRLFSVGPGSNLPAIREKYTQHKILHAADVIDLNMANAFKNVKILCQCPCQCNLLEEVMLKLFPYWKLSTAV

SEQ ID NO: 44 - DNA, 1473 bp, Oryza sativa
  source 1..1473: mol_type = genomic DNA
atggctggaaggaaggaaaatccggtgcttactgcttgccaagcacccagtggtcgaatcacacgagctcaagctgctgcaaatcgtggacggtttgggtttgctccctccgtatcactacccgcaagaactgaacgaaagcagacagcaaaaggaaagacaaaaaggggagctttggatgaaatcactagtgcaagtactgcaacttcagctcctcagcctaaacggcgcacagtgctcaaggatgtaaccaacatcggctgtgccaactcatccaaaaattgcaccaccacgagcaagctgcagcaaaagtcaaagcccacccaaagggtgaaacaaatcccgagcaaaaagcagtgtgcaaagaaggttcctaagctaccccctccggctgttgctggaacttcatttgtgattgattctaaaagttctgaagaaactcaaaaggtggagcttttggcaaaagcagaggaacccacaaatttgtttgaaaacgaggggttactgtcattgcagaatattgagcgaaacagggacagtaattgccatgaggcattctttgaggcaagaaacgccatggataaacatgaactcgctgactccaagcctggtgactctagtggtttaggttttatagatattgacaatgataatggaaatcctcaaatgtgtgcttcctatgcttcagagatatacacaaatctgatggcctctgagcttatcagaagacccaggtcaaattacatggaggctttgcaacgtgacatcacaaagggcatgcgaggcattctcattgattggcttgttgaggtttctgaagaatataagcttgtgccagacacactctacctaaccattaatcttattgaccgatttctttctcaacattatattgaaagacagaaactccaacttcttggaataacaagcatgctgattgcctcgaaatatgaagagatatgtgctcctcgtgttgaagaattttgtttcataactgacaatacatacacaaaagctgaggtgctgaaaatggagggcctggtgcttaatgatatggggtttcatctatctgttccaacaacaaaaacatttctcaggagattccttagagccgcacaggcttctcgtaatgttccttcaattaccttgggatatctggccaattatcttgcagagctgaccctgatcgattacagtttcctcaaatttcttccttcagtggtggcagcatctgcagtctttcttgcaagatggacacttgaccaatctgacattccatggaatcatactcttgagcactacacttcttacaaaagctctgatattcaaatatgtgtctgtgctctacgggaactgcagcataacaccagtaattgccctctcaatgctatacgtgaaaagtataggcaacaaaagtttgagtgtgtagccaacctgacatcaccggagctggggcagtcactcttcagctga

SEQ ID NO: 45 - protein, 490 aa, Oryza sativa
MAGRKENPVLTACQAPSGRITRAQAAANRGRFGFAPSVSLPARTERKQTAKGKTKRGALDEITSASTATSAPQPKRRTVLKDVTNIGCANSSKNCTTTSKLQQKSKPTQRVKQIPSKKQCAKKVPKLPPPAVAGTSFVIDSKSSEETQKVELLAKAEEPTNLFENEGLLSLQNIERNRDSNCHEAFFEARNAMDKHELADSKPGDSSGLGFIDIDNDNGNPQMCASYASEIYTNLMASELIRRPRSNYMEALQRDITKGMRGILIDWLVEVSEEYKLVPDTLYLTINLIDRFLSQHYIERQKLQLLGITSMLIASKYEEICAPRVEEFCFITDNTYTKAEVLKMEGLVLNDMGFHLSVPTTKTFLRRFLRAAQASRNVPSITLGYLANYLAELTLIDYSFLKFLPSVVAASAVFLARWTLDQSDIPWNHTLEHYTSYKSSDIQICVCALRELQHNTSNCPLNAIREKYRQQKFECVANLTSPELGQSLFS

SEQ ID NO: 46 - DNA, 1527 bp, Oryza sativa
  source 1..1527: mol_type = genomic DNA
atgtcgagcaacctagcagcctcccgccgctcgtcgtcgtcgtcctcggtggcggcggcggcggcggcgaagcgacccgcggtgggggagggaggaggaggaggaggagggaaggcggcagcgggcgccgccgcggcaaagaagcgcgtggcgcttagcaacatcagcaacgtcgccgctggtggtggcgccccagggaaggccggcaatgcgaagttgaatttagctgcctcagctgcaccagtgaagaagggatctttggccagtggccgcaatgtgggcacgaatcgggcctcggcggtgaaatcggcttccgccaagccggctccggccatatcccgccatgagagcgccacacagaaggagtctgttcttcctcctaaagtgcctagcattgtgccgactgctgcactggcacctgtcactgtaccctgcagcagcttcgtctcccctatgcattcaggagattcagtttcggttgacgagacgatgtcgacgtgtgactcaatgaaaagcccagaatttgagtacattgataatggggattcctcctcagttctaggttccttgcagcgaagagcaaacgaaaacctgcgtatctcagaggatagagatgtcgaagaaactaagtggaagaaggatgctccttccccaatggaaatcgaccaaatttgtgatgttgacaataactacgaggatccgcagttgtgtgctactcttgcttctgatatctacatgcacttgcgcgaggctgagaccaggaaacgtccatcaactgattttatggaaacaatccaaaaggatgtaaacccaagcatgagagcgatcctgatagactggcttgtggaagtcgctgaagaatatcgtcttgttcctgatacattatacctgacagttaactacattgatcgttatctttctggcaatgagatcaatcgtcaaagactgcaattacttggagttgcttgtatgcttattgctgcaaaatacgaggagatatgtgcacctcaagtagaagaattctgctatataactgacaacacatacttcagagatgaggttttggaaatggaagcttctgtcctgaattacctgaagtttgaagtgactgcacctacagcaaaatgctttttgaggagatttgtccgtgttgcacaagtatcggatgaggatccagcattgcatcttgagttcctagccaattatgttgctgagctatcactgctggagtacaatctactttcttaccctccttcactagtagcggcatcggctattttcttggccaaattcatactgcagccaacaaagcacccttggaattccacccttgctcactacacacaatacaagtcgtcagagttaagcgactgtgtaaaggcattgcaccgcctttttagcgttggtcccgggagtaaccttcctgcaatcagggagaagtatacccaacataagtacaaatttgtggcgaagaagccctgcccaccctcaataccgaccgaattctttcgcgacgcaacatgctga

SEQ ID NO: 47 - protein, 508 aa, Oryza sativa
MSSNLAASRRSSSSSSVAAAAAAKRPAVGEGGGGGGGKAAAGAAAAKKRVALSNISNVAAGGGAPGKAGNAKLNLAASAAPVKKGSLASGRNVGTNRASAVKSASAKPAPAISRHESATQKESVLPPKVPSIVPTAALAPVTVPCSSFVSPMHSGDSVSVDETMSTCDSMKSPEFEYIDNGDSSSVLGSLQRRANENLRISEDRDVEETKWKKDAPSPMEIDQICDVDNNYEDPQLCATLASDIYMHLREAETRKRPSTDFMETIQKDVNPSMRAILIDWLVEVAEEYRLVPDTLYLTVNYIDRYLSGNEINRQRLQLLGVACMLIAAKYEEICAPQVEEFCYITDNTYFRDEVLEMEASVLNYLKFEVTAPTAKCFLRRFVRVAQVSDEDPALHLEFLANYVAELSLLEYNLLSYPPSLVAASAIFLAKFILQPTKHPWNSTLAHYTQYKSSELSDCVKALHRLFSVGPGSNLPAIREKYTQHKYKFVAKKPCPPSIPTEFFRDATC

SEQ ID NO: 48 - DNA, 1158 bp, Oryza sativa
  source 1..1158: mol_type = genomic DNA
atggctgacaaggagaactccaccccggcctccgcggcgcggctcacccgctcgtctgcggcggctggggcgcaggccaagcgttcggccgccgcgggcgtcgccgacggtggcgcgccgccggcgaagaggaagcgcgtcgcgctcagcgacctcccgaccctctccaacgccgtcgtcgtcgccccccgccagccgcaccaccccgtcgtcatcaagccgtcgtccaagcagcccgagcccgccgcggaggcggcggcgcccagcggcggcggcggcggctcccccgtgtcatccgcgtcgacgtcgacggcgtcgccctcctccggttgggacccgcagtacgcctccgacatctacacctacctccgatccatggaggtggaggcgcggaggcagtcggcggcggactacatcgaggcggtgcaggtggacgtgacggcgaacatgcgggccatcctcgtggactggctggtggaggtcgccgacgagtacaagctcgtcgccgacacgctctacctcgccgtctcctacctcgaccgctacctctccgcccacccgctcaggcgcaacaggctgcagctcctcggcgtcggcgccatgctcatcgctgcgaagtacgaggagattagccctcctcatgtggaggatttctgctacatcactgataatacgtacactaggcaggaggttgtcaagatggagagcgacatactcaagcttctcgagttcgagatgggcaatcctaccatcaagacattcctcaggcggttcacgagatcttgccaggaagacaaaaagcgctccagcttgttattggagttcatggggagttatcttgctgagcttagtctacttgactacggctgtctccggttcttgccatcggtggttgctgcctcagtggtgtttgttgctaaactgaacattgatccgtacaccaatccttggagcaagaagatgcagaagttgacaggatacaaggtgtctgaactgaaggattgcatcttggccattcatgacttgcagctcagaaaaaaatgttcaaacttaactgcaattcgcgacaagtacaagcaacacaagttcaagtgtgtctcaacattgcttccccctgttgatatccctgcgtcatacctccaagatttaacagagtag

SEQ ID NO: 49 - protein, 385 aa, Oryza sativa
MADKENSTPASAARLTRSSAAAGAQAKRSAAAGVADGGAPPAKRKRVALSDLPTLSNAVVVAPRQPHHPVVIKPSSKQPEPAAEAAAPSGGGGGSPVSSASTSTASPSSGWDPQYASDIYTYLRSMEVEARRQSAADYIEAVQVDVTANMRAILVDWLVEVADEYKLVADTLYLAVSYLDRYLSAHPLRRNRLQLLGVGAMLIAAKYEEISPPHVEDFCYITDNTYTRQEVVKMESDILKLLEFEMGNPTIKTFLRRFTRSCQEDKKRSSLLLEFMGSYLAELSLLDYGCLRFLPSVVAASVVFVAKLNIDPYTNPWSKKMQKLTGYKVSELKDCILAIHDLQLRKKCSNLTAIRDKYKQHKFKCVSTLLPPVDIPASYLQDLTE

SEQ ID NO: 50 - DNA, 1122 bp, Oryza sativa
  source 1..1122: mol_type = genomic DNA
atggccggcaaggagaacgccgcggcggcgcagccccgcctcacccgcgccgccgccaagcgcgcggccgccgtcaccgccgtggccgtcgccgccaagcgcaagcgcgtcgcgctcagcgagctccccacgctgtccaacaacaacgccgtggtgctcaagccgcagccggcgcccaggggcggcaagagggccgcctcccacgccgccgagcccaagaagccagctccgccgccggcgccggcggtggtggtcgtggtcgacgacgacgaggagggggagggggatccgcagctctgcgcgccctacgcctccgacatcaactcctacctccgctccatggaggtgcaagcgaagcggcggccggcggcggactacatcgagacggtgcaggtggacgtgacggccaacatgcgaggcatcctggtcgactggctcgtcgaggtcgccgaggagtacaagctcgtctccgacacgctctacctcaccgtctcctacatcgaccgcttcctctccgccaaatccatcaaccgccagaagctgcagctcctcggcgtctccgccatgctcatcgcctcgaagtatgaggagatcagccccccaaatgtggaggatttctgctatataaccgacaatacctatatgaaacaggaggttgtcaagatggagcgcgatatactgaatgttctcaagtttgagatgggcaatcctacaaccaagacgttcctgaggatgttcatcagatctagccaagaagacgataagtatcctagccttcccttggagttcatgtgtagctatcttgccgagctgagcctgctggagtacggctgtgttcggctcttgccatccgttgttgcagcctcagtggtgtttgttgcaaggctaacccttgattcagacaccaatccttggagcaagaagttgcaagaggtgaccggctacagggcatctgagttgaaggattgcattacctgcatacatgacttgcagctaaacaggaaagggtcatctctaatggctatccgggacaagtacaagcaacataggttcaagggcgtatcaacattgttaccccctgttgagatccctgcatcatacttcgaagacctaaacgagtag

SEQ ID NO: 51 - protein, 373 aa, Oryza sativa
MAGKENAAAAQPRLTRAAAKRAAAVTAVAVAAKRKRVALSELPTLSNNNAVVLKPQPAPRGGKRAASHAAEPKKPAPPPAPAVVVVVDDDEEGEGDPQLCAPYASDINSYLRSMEVQAKRRPAADYIETVQVDVTANMRGILVDWLVEVAEEYKLVSDTLYLTVSYIDRFLSAKSINRQKLQLLGVSAMLIASKYEEISPPNVEDFCYITDNTYMKQEVVKMERDILNVLKFEMGNPTTKTFLRMFIRSSQEDDKYPSLPLEFMCSYLAELSLLEYGCVRLLPSVVAASVVFVARLTLDSDTNPWSKKLQEVTGYRASELKDCITCIHDLQLNRKGSSLMAIRDKYKQHRFKGVSTLLPPVEIPASYFEDLNE

SEQ ID NO: 52 - DNA, 1920 bp, Arabidopsis thaliana
  source 1..1920: mol_type = genomic DNA
atgagtagtacgatgttcgtgaaacggaatccgattagagaaaccaccgccgggaaaatctcttcgccgtcgtcaccgactttgaatgttgcagtcgcgcatataagagctggatcttattacgaaatcgatgcttcgattcttcctcagagatcgccggaaaatcttaaatcgattagagtcgtcatggtgagcaaaatcacggcgagtgacgtgtctctccggtacccaagcatgttttcactccgatcgcatttcgattacagtaggatgaaccggaataaaccgatgaagaagaggagtggtggtggtcttcttcctgttttcgacgagagtcatgtgatggcttcggagctagctggagacttgctttacagaagaatcgcacctcatgaactttctatgaatagaaattcctggggtttctgggtttctagttcttctcgcaggaacaaatttccaagaagggaggtggtttctcaaccggcgtacaatactcgtctctgtcgcgctgcttcaccggagggaaagtgctcgtctgagctgaaatcgggagggatgatcaagtggggaaggagattgcgtgtgcagtatcagagtcggcatattgatactaggaagaataaggaaggtgaggagagttctagagtgaaggatgaagtttacaaagaagaagagatggagaaagaagaggatgatgatgatgggaatgaaataggaggcactaaacaagaggcaaaggagataactaatggaaatcgtaagagaaagctgattgaatcaagtactgagagactcgctcagaaagctaaggtttatgatcagaagaaggaaactcaaattgtggtttataagaggaaatcagagaggaagttcattgatagatggtctgttgagaggtacaaactagctgagaggaacatgttaaaagtgatgaaggagaagaatgcagtgtttggcaactccatactcaggccagagttgaggtcagaagcaaggaagctgattggtgacacaggtctattggatcatctgcttaagcacatggctggtaaggtggctcctggaggtcaagataggtttatgagaaagcacaatgcagatggggcaatggagtattggttggagagttctgatttgattcacataaggaaagaagcaggagttaaagatccttactggactcctccacctggttggaagcttggtgacaacccttctcaagatcctgtctgcgctggagaaatccgtgacatcagagaagaattagctagcctgaaaagagaattgaagaaacttgcgtcaaagaaggaagaggaggagcttgttatcatgactacgcctaattcttgtgttactagtcagaatgataatctgatgactccagcaaaggaaatctacgctgatctgctgaaaaagaaatacaaaattgaggaccagctagtgattattggagaaaccttgcgtaaaatggaggaagacatgggatggcttaagaaaacagtggacgagaactatcctaaaaagccagactcaacagagacacctttgctactagaggattcaccaccaatacagacactagaaggagaagtgaaggtggtgaacaagggtaaccaaatcacagagtcacctcaaaacagagaaaaaggaaggaagcatgatcaacaagaaagatcaccactttcactaataagcaacactggtttcagaatctgcaggcctgtggggatgttcgcatggccccaattgcctgctcttgctgctgctactgatactaatgcttcttcgccaagtcacagacaagcctacccatccccttttccagtcaagccacttgcagctaagcgtcctcttggcttgacgtttcccttcaccatcatacccgaagaagctcccaagaatctcttcaacgtttga

SEQ ID NO: 53 - protein, 639 aa, Arabidopsis thaliana
MSSTMFVKRNPIRETTAGKISSPSSPTLNVAVAHIRAGSYYEIDASILPQRSPENLKSIRVVMVSKITASDVSLRYPSMFSLRSHFDYSRMNRNKPMKKRSGGGLLPVFDESHVMASELAGDLLYRRIAPHELSMNRNSWGFWVSSSSRRNKFPRREVVSQPAYNTRLCRAASPEGKCSSELKSGGMIKWGRRLRVQYQSRHIDTRKNKEGEESSRVKDEVYKEEEMEKEEDDDDGNEIGGTKQEAKEITNGNRKRKLIESSTERLAQKAKVYDQKKETQIVVYKRKSERKFIDRWSVERYKLAERNMLKVMKEKNAVFGNSILRPELRSEARKLIGDTGLLDHLLKHMAGKVAPGGQDRFMRKHNADGAMEYWLESSDLIHIRKEAGVKDPYWTPPPGWKLGDNPSQDPVCAGEIRDIREELASLKRELKKLASKKEEEELVIMTTPNSCVTSQNDNLMTPAKEIYADLLKKKYKIEDQLVIIGETLRKMEEDMGWLKKTVDENYPKKPDSTETPLLLEDSPPIQTLEGEVKVVNKGNQITESPQNREKGRKHDQQERSPLSLISNTGFRICRPVGMFAWPQLPALAAATDTNASSPSHRQAYPSPFPVKPLAAKRPLGLTFPFTIIPEEAPKNLFNV

SEQ ID NO: 54 - DNA, 1254 bp, Oryza sativa
  source 1..1254: mol_type = genomic DNA
atgaccgccgccgccgtcaacgcctcgcgcgtcatgcgccgccgcgccgccggcgaggacctgggcgacggcgacttctgggccggtggcgccccgcgcctctacgacttctcccagcaggagcagaagccgttcttgccggcgccgccgtcgcccgcgcccgtcccggcgtcgccgccgtcccccgcggcggagtcggtggcgccgtgcctcctcacgctgcagtgcagcggcgtcgggtggggggtcaggaagcgggtcaggtacgtcgggaggcaccaccacctcgcgcgccaccacgctcccgaacgcgccgtggacgccgcgcgggacgacgacgaggcgagctccgccaaggccaagaacgagagcccgaaggaggaggcggcggcggcagaagaagacgacgacgtcgaacacaaggtggcggtgcgcaccacgtcggaggagaagaagaagaagaggaggaggaagcgcggccgtggccgtgtccgtggccatggcgtcgccaagcgtcccaagaaggaggatgaggaggggacgaagctctcggctcccaaggccgagcagctcgaggaggaggaggagggcgccgcggtggcggcgccgagcggcatgatcgaccggtggaaggcgacccggtacgccaccgcggaggcgtcgctgctcgccatcatgcgcgcccacggcgcgcgcgccgggaagcccgtcccgcgcgcggcgctgcgggaggaggcgcgcgcccacatcggcgacacgggcctcctcgaccacctcctcaggcacatcgccgacaaggtggcgcccggcggcgccgagcggttccggcggcggcacaacgccggcggcgggctggagtactggctggagcccgccgagctcgccgccgtgcggcggaaggccggcgtggccgacccgtactgggtgcctcctcccggatggaagccaggggaccccgtgtcgccggagggctacttgctggaggtgaggaagcaggtggagcagctcgccgttgagctcgccggcgtcagaaggcacatggatcacctcacttccaatgtgagtcaagtgggcaaggaaatcaaatctgaggctgagaagtcctacaatacatgtcagggtggggacccaccctaccttgaccggatctcgatccgtgccttcgcccggaagctaagccgcagctcgtccttgagcgggacccagcgccgtcccccgcctgacacgggggacagccctgtcattccctcataa

SEQ ID NO: 55 - protein, 417 aa, Oryza sativa
MTAAAVNASRVMRRRAAGEDLGDGDFWAGGAPRLYDFSQQEQKPFLPAPPSPAPVPASPPSPAAESVAPCLLTLQCSGVGWGVRKRVRYVGRHHHLARHHAPERAVDAARDDDEASSAKAKNESPKEEAAAAEEDDDVEHKVAVRTTSEEKKKKRRRKRGRGRVRGHGVAKRPKKEDEEGTKLSAPKAEQLEEEEEGAAVAAPSGMIDRWKATRYATAEASLLAIMRAHGARAGKPVPRAALREEARAHIGDTGLLDHLLRHIADKVAPGGAERFRRRHNAGGGLEYWLEPAELAAVRRKAGVADPYWVPPPGWKPGDPVSPEGYLLEVRKQVEQLAVELAGVRRHMDHLTSNVSQVGKEIKSEAEKSYNTCQGGDPPYLDRISIRAFARKLSRSSSLSGTQRRPPPDTGDSPVIPS

SEQ ID NO: 56 - DNA, 1914 bp, Oryza sativa
  source 1..1914: mol_type = genomic DNA
atgaccgccgccgccgtcaacgcctcgcgcgtcatgcgccgccgcgccgcaggcgaggacctgggcgacggcggcgatggcgacggcgacttctgggccggtggcgccccccgcctctacgacttctcccagcaggagcagaagccgttcttgcccgcgcccgcgcccgcgccgccgtcgcccgcgcccgtcccggcgtcgccgccgtcccccgcggcggagtcggtggcgccgtgcctcctcacgctgcagtgcagcggcgtcgggtggggtgtcaggaagcgggtccggtacgtcgggaggcaccaccacctcgcgcgccaccacgctcccgagcgcgccgtggacgccgcgcgggacgacgacgaggcgagctccgccaaggccaagaacgagagcccgaaggaggaggcagcggcggcagaagaagacgacgacaacgtcgaacacaaggtggcggtgcccaccacgtcggaggagaagaagaggaggaggaggaggaagcgtggccgtggccgtgtcggtggccatggcgtcgccaagcgtcccaagaaggaggaggaggaggaggagacgaagctctcggctcccaaggccgagcagctcgaggaggaggagggcgccgcggtggcggcgccgagcggcatgatcgaccggtggaaggcgacccggtacgccaccgcggaggcgtcgctgctcgccatcatgcgcgcccgcggcgcgcgcgccgggaagcccgtcccgcgcggggcgctgcgggaggaggcgcgcgcccacattggcgacacgggcctcctcgaccacctcctcaggcacatcgccgacaaggtggcgcccggcggcgccgagcggttccggcggcggcacaacgccggcggcgggctggagtactggctcgagcccgccgagctcgccgccgtgcggcggaacgccggcgtggccgacccgtactgggtgcctcctcccggatggaagccaggggaccccgtctcgccggagggctacttgctggaggtgaggaagcaggtggagaagctcgccgtggagctcgccggcgtcagaaggcacatggatcacctctcttccaatgtgagtcaagtgggcaaggaaatcaaatctgaggctgagaaatcctacaatacatgccaggagaagtatgcctgtatggagaaagccaatggcaatctggaaaagcagcttctgtccttggaggagaagtatgagaatgcaacacacgcaaatggcgagctgaaggaggagttgttgtttctcaaggagaagtttgtgagtgtggtcgagaacaacaccagactggagcaccagctgactgctttatccacttctttcctgtctctaaaggaggaactgctctggctggaaaaagaagaagctgatctgtatgtcaaggaaccatgggaagacgacgatgaaaagcaagaacacgatgccgggaaagaggcgaaggacgacgatgtcgccggcgtcagtgcagccaacgaccagccggacgtcgacggcgatggcaccaccaccaccaccaccaccagcagcaatggtggcagcgggaagagaacatcgaggaagtgcagcgtgcgcatctccaagccgcagggcgcgttccagtggccgacgccgagcctgccgttctcgccggagctcgccgcgccgccgtcgccgccgctgaccccgacggcgcccgtcgtcgccggcgccgccaacttcgccaccatggacgagctctacgagtacatgatggccggcggcctccccacgccaccgtccaccaccagcaacgccgggaagctcccctcgctgcccgccgccacggcctgcgccacgacgccgccggtgaagacggcggacgccgccggcgacgtgggcaccgagctggcactggccactcccgcctactga

SEQ ID NO: 57 - protein, 637 aa, Oryza sativa
MTAAAVNASRVMRRRAAGEDLGDGGDGDGDFWAGGAPRLYDFSQQEQKPFLPAPAPAPPSPAPVPASPPSPAAESVAPCLLTLQCSGVGWGVRKRVRYVGRHHHLARHHAPERAVDAARDDDEASSAKAKNESPKEEAAAAEEDDDNVEHKVAVPTTSEEKKRRRRRKRGRGRVGGHGVAKRPKKEEEEEETKLSAPKAEQLEEEEGAAVAAPSGMIDRWKATRYATAEASLLAIMRARGARAGKPVPRGALREEARAHIGDTGLLDHLLRHIADKVAPGGAERFRRRHNAGGGLEYWLEPAELAAVRRNAGVADPYWVPPPGWKPGDPVSPEGYLLEVRKQVEKLAVELAGVRRHMDHLSSNVSQVGKEIKSEAEKSYNTCQEKYACMEKANGNLEKQLLSLEEKYENATHANGELKEELLFLKEKFVSVVENNTRLEHQLTALSTSFLSLKEELLWLEKEEADLYVKEPWEDDDEKQEHDAGKEAKDDDVAGVSAANDQPDVDGDGTTTTTTTSSNGGSGKRTSRKCSVRISKPQGAFQWPTPSLPFSPELAAPPSPPLTPTAPVVAGAANFATMDELYEYMMAGGLPTPPSTTSNAGKLPSLPAATACATTPPVKTADAAGDVGTELALATPAY

SEQ ID NO: 58 - DNA, 2412 bp, Oryza sativa
  source 1..2412: mol_type = genomic DNA
atggacgcggagatggcggctcctgcgcttgcggcagctcatctgctggactcgcccatgaggccacaggtgagcagatactactccaagaagaggggtagcagccacagcagaaatggcaaggatgatgccaaccatgacgagtccaagaaccaatcacccggcttgcccctgagcagacagagcctgtcctcatctgccacccacacctaccacaccggagggttctacgagatcgaccacgagaagcttccccccaaatccccaattcatctcaagtccatacgcgtggtaaaggtgagcggctacacaagcctggacgtcacagtgagcttcccgtccctcctggcgctgcgaagcttcttctcctcctccccacggtcgtgcactgggccggagctcgacgagcgcttcgtcatgagcagcaaccacgcggcccgcatcctgcgccgtcgggtggccgaggaggagctcgcgggcgacgtgatgcaccaggacagcttctggctcgtcaagccctgcctctatgacttctccgcgtcgtcaccacatgatgtgctgaccccgtcgccgccgcctgccacagcgcaggcgaaggcgccggcagccagttcctgccttctcgacaccttgaagtgcgacggcgccgggtggggcgtgaggcgccgtgtcaggtacattggtcgccaccacgatgcttccaaggaggccagcgctgccagcctcgatggctacaacacagaggtcagcgtccaggaggagcagcagcagcgactgcggcttcgactgcggttgcgacaacgccgggagcaggaagacaacaagagcactagcaatggcaagaggaagcgggaggaggcagagagcagcatggacaagagcagagccgccaggaagaagaaagccaagacttacaagagtcccaagaaggtggagaagaggcgcgtcgtggaggctaaagacggcgaccctcggcgcggcaaggaccggtggtcggccgagcggtacgcagcggcggagaggagcctgctggatataatgcgctcccatggtgcctgcttcggtgcgccggtgatgcggcaggctctgcgggaggaagcccgcaagcatatcggtgacaccggcctccttgaccacctgctcaagcacatggccggcagggtaccggaaggcagcgcggaccggttccgtcgccggcacaatgcggatggtgccatggagtactggctggagccggcggagcttgccgaggtacggcggctggctggagtgtctgatccatactgggtgccgccacctgggtggaagccaggtgatgacgtgtccgcagtcgccggtgacctcctggtcaagaagaaggtggaagagctcgctgaggaggttgatggtgtaaaaaggcacatcgagcagctcagttctaatttggtgcagctggagaaggaaacaaaatctgaggcagagcgatcttacagctctaggaaggagaagtatcagaagttgatgaaggcaaatgaaaagctcgagaaacaggtgttatctatgaaggacatgtatgagcatctggttcagaaaaagggtaagctgaagaaggaggtgctgtccttgaaggataaatacaagcttgtgctggagaagaatgataaactggaggaacagatggctagtctctccagctccttcctttctttgaaggaacaattgctgctgccaagaaatggagataatctgaacatggaaagggaaagggtggaagtgactttgggcaagcaagaaggccttgttcccggcgaaccactgtatgttgatggtggtgaccggatcagccagcaagcagatgccaccgtcgtccaagtcggcgagaagaggacggcgaggaagagcagcttccgcatctgcaagccacagggaacgttcatgtggccacacatggcgtctggcacgagcatggccatcagtgggggaggcagcagcagctgccctgtcgcctccgggccagagcagctccctcgcagcagcagctgccccagcattgggcctggtggcctcccgccgtcgtcacgagccccagccgaggtggtggtcgcgtcgccactggacgagcacgtggcgttccgcgggggcttcaacacgccgccctcggcatcgtccaccaacgccgccgctgccgccaagctgcctcccctgcccagcccgacgtcacctctccagacacgggccctgttcgccgctggcttcactgtcccggcattacacaacttctccggcctcaccttacgccatgtggactcctcgtcgccgtcgtccgcgccatgcggtgctagggagaagatggtgaccctgttcgatggagactgccgggggatcagcgtcgtgggcaccgagctggcactggccactccgtcctactgctga

SEQ ID NO: 59 - protein, 803 aa, Oryza sativa
MDAEMAAPALAAAHLLDSPMRPQVSRYYSKKRGSSHSRNGKDDANHDESKNQSPGLPLSRQSLSSSATHTYHTGGFYEIDHEKLPPKSPIHLKSIRVVKVSGYTSLDVTVSFPSLLALRSFFSSSPRSCTGPELDERFVMSSNHAARILRRRVAEEELAGDVMHQDSFWLVKPCLYDFSASSPHDVLTPSPPPATAQAKAPAASSCLLDTLKCDGAGWGVRRRVRYIGRHHDASKEASAASLDGYNTEVSVQEEQQQRLRLRLRLRQRREQEDNKSTSNGKRKREEAESSMDKSRAARKKKAKTYKSPKKVEKRRVVEAKDGDPRRGKDRWSAERYAAAERSLLDIMRSHGACFGAPVMRQALREEARKHIGDTGLLDHLLKHMAGRVPEGSADRFRRRHNADGAMEYWLEPAELAEVRRLAGVSDPYWVPPPGWKPGDDVSAVAGDLLVKKKVEELAEEVDGVKRHIEQLSSNLVQLEKETKSEAERSYSSRKEKYQKLMKANEKLEKQVLSMKDMYEHLVQKKGKLKKEVLSLKDKYKLVLEKNDKLEEQMASLSSSFLSLKEQLLLPRNGDNLNMERERVEVTLGKQEGLVPGEPLYVDGGDRISQQADATVVQVGEKRTARKSSFRICKPQGTFMWPHMASGTSMAISGGGSSSCPVASGPEQLPRSSSCPSIGPGGLPPSSRAPAEVVVASPLDEHVAFRGGFNTPPSASSTNAAAAAKLPPLPSPTSPLQTRALFAAGFTVPALHNFSGLTLRHVDSSSPSSAPCGAREKMVTLFDGDCRGISVVGTELALATPSYC

SEQ ID NO: 60 - DNA, 495 bp, synthetic construct
  source 1..495: mol_type = other DNA
gagatttgggaaatgtgcaatttgggtttatctggttttgttgttttggttatttagttttgagtccggtttgaagaaatgcttcatagatatataaatgtaaccagaaatataaataaatcataactaagtagtattatatttttgttaagcttaattatttcaattccaagtctttcctaagaatttgttgaaaatttataatttacgttacactttgtaaaatcagaacgatccaattcacaagattaatgctacgactctgtttttttctttaaaaataaatcaataatcatctccactaaacctctcaataacttagcagtcttaatgaaatttaaagctaatctatcaatcacattctcaccacgtcgccaaactcgttgccgtttcaatcttttcaagttccctttcatgctgtttataaacccttgcactctttcactcacagactcactacaagtctacaccacaaacttaccaaatcatccaaaa

SEQ ID NO: 61 - DNA, 1890 bp, synthetic construct
  source 1..1890: mol_type = other DNA
acacgttatggaaagcaaagaataacaaaagtaatattcttacctatcttttttagttggaaaacttgcattgtgtaacgtattcaaacattttcgaaatggtttattggtttttgtatataattaatttgggttaaactgatatattttatgagataaatatagaatctcatgtgcttatacaaagcaactatattaattttgttaaccgtaagttacaaaaacagtggtcggaggaaatcaggaaaataaaaagagagaaaagagtctacacaatgggccaattattataagtaaatgatagtcatgaaagcccatttcagaagaagatcttttggaaatgagaatagtgctctaggctcactggtcctttttactattggtatagaaactgtcaaagcccaacaggtttaaactagcatttcaggcgctgtaattcttctgcagtttgtttgtataaacttggaatatggatgggttaaacactgatatttcttcactcgttttgtctcacatactgttcgatgcttaacacaggtcttaaaaagaaactgggtttgatgtctcaaatctactcaagaaagaaagatatcttgagtttgcatcgagacagaaaagagtacgactatacatagctgctggggtagtagcctgcagagaatacagattttgaaccacgtacaaggaaccaaatcagtgtatgtatctaactattaaccttgtggtgtgatcttgtcctcttaggtattgtggaatccttgtaggaaatgtcatggcaactcattagtcatcttgaaccaaatgagatgatacatgatggtctcaaattggacatggtggcaccttttgtttcgtgagtggctttcaatttatctccatagaaattgtttaattttcgttattggtgcctgtcaataaaaattacaaacatatgcagagcgttggattcgtggatcgttgaacatcctattgagagacagggccagcctcctaattgtatgacatcgtctctttcacaatatactcattaaatgagaggttgagatttgacttatttgctttatacagcctgcacagtgtggaagacccctctaaagactgaactggggacagcaacaatgggaatctgaccatcctcatgacagtacctggaaagagtctcagaagcttcaagttcagtacgcagcttgaccagtctttcagtcatatagccataggggttgaattagtgtccatcttcccattgtgattaacgttctgatttagcatgcaccttcgaattaagtgaatctattaccatgtgaccaagccattgcattactaatataagcatatcacatttcccttttctccgtgccaactgaatttgaattattttccctcaacttaatcacatgttttcctcacggccaaaagtactctcagtgttacatgattaccacaacaaatgatttaaactttgaacttttgaagttatcgagcaacatggcaaatcctggtcctatatgacataacatgagttcctctgcctattgtaaaattaggaaacacaaaaccaaaatgattatatctggtattatagtgtggtgtataacatatactcacacaagatatgctcttaagatgataaatgtctaatcttccaagtcccaattttgaaaacgttgatattaatttcccctcaaccccactagcctcaaattaaattagcagccttagtgtgaaattaaaagatagctaatgaattgcatttcagactttcacctccccactcacgtagctataactccttaccgtttcaaatctcttcacttccccaattttgttgtgtataaaaacctcttctccacttcactctttccaccacaaactttctaaaactaatcaaca

SEQ ID NO: 62 - DNA, 20 bp, synthetic construct
  source 1..20: mol_type = other DNA
gagaaattccggcggtaggg

SEQ ID NO: 63 - DNA, 20 bp, synthetic construct
  source 1..20: mol_type = other DNA
gcgctcgccgacccctcggg

SEQ ID NO: 64 - DNA, 20 bp, synthetic construct
  source 1..20: mol_type = other DNA
tcgacgacaacctcctcacc

SEQ ID NO: 65 - DNA, 20 bp, synthetic construct
  source 1..20: mol_type = other DNA
gtgtggcgatcgtgtacgag

(19) United States
(12) Patent Application Publication
(10) Pub. No.: US 2026/0366035 A1
(43) Pub. Date: Nov. 19, 2026

(54) CIRCUMVENTING BARRIERS TO HYBRID CROPS FROM GENETICALLY DISTANT CROSSES

(71) Applicants: The Regents of the University of California, Oakland, CA (US); Centre de Cooperation Internationale en Recherche Agronomique pour le Developpement, Paris (FR); Max-Planck-Gesellschaft zur Foerderung der Wissenschaften e.V., Munich (DE)

(72) Inventors: Venkatesan SUNDARESAN, Davis, CA (US); Imtiyaz KHANDAY, Davis, CA (US); Emmanuel GUIDERDONI, Montpellier (FR); Delphine MIEULET, Montpellier (FR); Raphael MERCIER, Munich (DE)

(21) Appl. No.: 19/118,280

(22) PCT Filed: Oct. 2, 2023
(86) PCT No.: PCT/IB2023/000562

§ 371 Date: Apr. 3, 2025

Related U.S. Application Data
(60) Provisional application No. 63/412,667, filed on Oct. 3, 2022.

(51) Int. Cl. C12N 15/82; C07K 14/415

(52) U.S. Cl. CPC C12N 15/8233; C07K 14/415; C12N 15/8287

(57) ABSTRACT

Synthetic apomixis can be achieved in an F1 hybrid of rice by inducing MiMe mutations and egg cell expression of BBM1 in a single step, while simultaneously providing an increased efficiency of clonal seed production. This can be used to generate hybrid plants from crosses from distant subspecies parents that produce clonal seed, allowing for maintenance of heterosis in future generations.

### CROSS-REFERENCE TO RELATED PATENT APPLICATIONS

The present patent application claims benefit of priority to U.S. Provisional Patent Application No. 63/412,667, filed Oct. 3, 2022, the entirety of which is hereby incorporated by reference for all purposes.

### BACKGROUND OF THE INVENTION

Heterosis, also termed hybrid vigor, refers to the higher performance of a hybrid progeny in comparison to those of its parents. Several underlying mechanisms (dominance, overdominance, and epistasis) have been proposed to explain the genetic and molecular bases of heterosis¹. Heterosis has been harnessed in crops, notably through the development of F1 hybrids in seed crops that exhibit superior yield potential and stability. However, F1 hybrid seeds must be renewed at every crop season because F2 progeny seeds are prone to trait segregation and lower global plant performance. Rice, the staple food for more than half of mankind, has a naturally low outcrossing rate and the production of F1 hybrid seeds relies on the implementation of complex male sterility systems², resulting in a high seed cost. Thus, the dissemination of hybrid rice is essentially restricted to areas with efficient and well-established seed production and distribution systems. As a result, the benefits of hybrids have not yet reached a large number of rice farmers, and the higher production potential, enhanced stability under environmental fluctuations, and lower input requirements of hybrids remain largely unexploited.

A potentially revolutionary alternative to male sterility systems is the propagation of F1 seeds over generations in an immortalized manner through an asexual, clonal mode of reproduction called apomixis. Apomixis occurs naturally in more than 120 genera of angiosperms, notably in some wild relatives of crops such as maize, wheat, and millet³. However, attempts to find naturally occurring apomixis in crops or to transfer the genetically characterized apomictic loci from wild relatives to crops have so far failed⁴.

Although harnessing naturally occurring apomictic mechanisms for crops has so far been unsuccessful, recently, the possibility of engineering synthetic apomixis in plants has emerged^{5,6}. Synthetic apomixis recapitulates a natural mode of apomixis, which is characterized by the formation of an unrecombined and unreduced diploid egg cell followed by its parthenogenetic development, leading to a clonal diploid zygote⁷. In synthetic apomixis, meiosis is converted to mitosis through a set of three mutations (called MiMe for Mitosis instead of Meiosis^{8,9}) that target the three features that differentiate meiosis from mitosis: First, recombination and pairing are suppressed through the inactivation of a member of the recombination initiation complex (e.g. SPO11-1¹⁰ or PAIR1¹¹). Second, the joint migration of sister chromatids at meiosis is replaced by their separation through the inactivation of the cohesin REC8¹². In the last step, the second meiotic division is omitted through the inactivation of the cell cycle regulator OSD1¹³. Using MiMe as a platform, three strategies have been used to induce unreduced and unrecombined egg cells to develop into diploid clonal embryos: i. crossing MiMe with a cenh3 mutant line expressing a CENH3-variant protein¹⁴ that induces paternal genome elimination in the zygote¹⁵; ii. inactivating the sperm cell-expressed phospholipase gene (NLD/MATL/PLA1)¹⁶ in MiMe¹⁷ that likely also contributes to paternal genome elimination¹⁸, or iii. expressing a parthenogenetic trigger in the egg cell¹⁹. The first strategy, implemented in Arabidopsis thaliana, requires a crossing step and is, therefore, a non-autonomous system as it cannot be propagated by self-fertilization. In the second strategy, the NLD/MATL/PLA1 mutation was found to alter plant fertility in rice (the fertility of the pair1/osrec8/ososd1/osmat1 quadruple mutant is 10% of that of the control MiMe), and the frequency of clonal seeds in the resulting apomictic plants remained low (6-8%)¹⁷. In the third strategy, synthetic apomictic plants were generated through the induction of the triple MiMe mutation by CRISPR/Cas9 in a rice line ectopically expressing the rice BABYBOOM1 (OsBBM1) AP2 family transcription factor specifically in egg cells¹⁹. Fertile apomictic rice plants produced clonal seeds at a rate of 10-30%, a frequency that remained stable over more than seven generations²⁰.

### BRIEF SUMMARY OF THE INVENTION

In some embodiments, methods of generating progeny of a subspecies plant cross is provided. In some embodiments, the method comprises,
providing F1 progeny of a subspecies plant cross;
transforming tissue of the F1 progeny with a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype;
selecting transformed cells from the tissue;
regenerating tissue from the transformed cells to form a transformed plant; and
collecting the selfed-seed from the regenerated plant.

In some embodiments, the tissue is embryonic or somatic tissue. In some embodiments, the fertility rate of plants from the selfed-seed is at least 20% (e.g., at least 25, 30, 40, 50%) higher than the fertility rate of F1 plants, measured by production of F2 progeny, from a natural subspecies cross, wherein the fertility rate is the percent of ovules that produce viable seeds.

In some embodiments, the fertility rate of plants from the selfed-seed is at least 80% (e.g., at least 90% or at least 95%), wherein the fertility rate is the percent of ovules that produce viable seeds.

In some embodiments, the subspecies are rice, barley, wheat or maize subspecies.

In some embodiments, the subspecies from the subspecies plant cross are indica and japonica, aus and japonica, or indica and aus.

In some embodiments, the first plant and the second plant are classified as different species. In some embodiments, a first plant of the subspecies plant cross is a wild plant and a second plant of the subspecies plant cross is domesticated.

In some embodiments, the Babyboom protein is at least 70, 80, 85, 90, 95, 98 or 100% identical to SEQ ID NO:1, 2, 3, 4, 5, 6, 7, 8, 9, 10, 11, 12, 13, 14, 15, 16, 17, 18, 19, or 20.

In some embodiments, the MiME expression cassettes comprise at least one expression cassette encoding a gRNA targeting (i) OSD1 or an ortholog thereof, (ii) ATREC8 or an ortholog thereof, and (iii) ATSPO11 or an ortholog thereof.

In some embodiments, the MiME expression cassettes comprise at least one expression cassette encoding a gRNA targeting (i) OSD1 or an ortholog thereof, (ii) REC8 or an ortholog thereof, and (iii) PAIR1 or an ortholog thereof.

Also provided is selfed-seed produced by the method as described above or elsewhere herein, or clonal progeny thereof.

Also provide is a plant clone of an F1 progeny plant from a subspecies plant cross, the plant clone comprising a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype, wherein the plant clone produces clonal seed. In some embodiments, the Babyboom protein is at least 70, 80, 85, 90, 95, 98 or 100% identical to SEQ ID NO:1, 2, 3, 4, 5, 6, 7, 8, 9, 10, 11, 12, 13, 14, 15, 16, 17, 18, 19, or 20. In some embodiments, the MiME expression cassettes comprise at least one expression cassette encoding a gRNA targeting (i) OSD1 or an ortholog thereof, (ii) ATREC8 or an ortholog thereof, and (iii) ATSPO11 or an ortholog thereof. In some embodiments, the MiME expression cassettes comprise at least one expression cassette encoding a gRNA targeting (i) OSD1 or an ortholog thereof, (ii) REC8 or an ortholog thereof, and (iii) PAIR1 or an ortholog thereof. In some embodiments, the plant is a rice, barley, wheat or maize plant.

Also provided is a method of generating a triploid fertile plant, the method comprising, generating triploid embryonic tissue comprising a heterologous a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype; regenerating tissue from the embryonic tissue to form a triploid plant; and collecting selfed-seed from the regenerated triploid plant, wherein the selfed-seed is triploid.

In some embodiments, the generating comprises crossing:
(a) a diploid parent plant comprising a heterologous a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype with
(b) a haploid parent plant comprising a heterologous a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype.

In some embodiments, the Babyboom protein is at least 70, 80, 85, 90, 95, 98 or 100% identical to SEQ ID NO:1, 2, 3, 4, 5, 6, 7, 8, 9, 10, 11, 12, 13, 14, 15, 16, 17, 18, 19, or 20. In some embodiments, the MiME expression cassettes comprise at least one expression cassette encoding a gRNA targeting (i) OSD1 or an ortholog thereof, (ii) ATREC8 or an ortholog thereof, and (iii) ATSPO11 or an ortholog thereof. In some embodiments, the MiME expression cassettes comprise at least one expression cassette encoding a gRNA targeting (i) OSD1 or an ortholog thereof, (ii) REC8 or an ortholog thereof, and (iii) PAIR1 or an ortholog thereof.

Also provided is a triploid selfed-seed produced by the methods described above or elsewhere herein, or clonal triploid progeny thereof.

Also provided is a triploid or higher ploidy fertile plant comprising a heterologous a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype, wherein the triploid or higher ploidy fertile plant produces selfed-seed that are triploid or higher ploidy, respectively. In some embodiments, the Babyboom protein is at least 70, 80, 85, 90, 95, 98 or 100% identical to SEQ ID NO:1, 2, 3, 4, 5, 6, 7, 8, 9, 10, 11, 12, 13, 14, 15, 16, 17, 18, 19, or 20. In some embodiments, the MiME expression cassettes comprise at least one expression cassette encoding a gRNA targeting (i) OSD1 or an ortholog thereof, (ii) ATREC8 or an ortholog thereof, and (iii) ATSPO11 or an ortholog thereof. In some embodiments, the MiME expression cassettes comprise at least one expression cassette encoding a gRNA targeting (i) OSD1 or an ortholog thereof, (ii) REC8 or an ortholog thereof, and (iii) PAIR1 or an ortholog thereof.

Also provided is method of generating clonal progeny of a plant (for example with at least 80%, 90%, or 95% efficiency). In some embodiments, the method comprises transforming tissue of the plant with a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype; selecting transformed cells from the tissue; regenerating tissue from the transformed cells to form a transformed plant; and collecting the selfed-seed from the regenerated plant, wherein the selfed-seed display a frequency of parthenogenesis of at least 80% (or 90% or 95%).

In some embodiments, the tissue is embryonic or somatic tissue. In some embodiments, the plant is a rice, barley, wheat or maize plant. In some embodiments, the Babyboom protein is at least 70, 80, 85, 90, 95, 98 or 100% identical to SEQ ID NO:1, 2, 3, 4, 5, 6, 7, 8, 9, 10, 11, 12, 13, 14, 15, 16, 17, 18, 19, or 20. In some embodiments, the MiME expression cassettes comprise at least one expression cassette encoding a gRNA targeting (i) OSD1 or an ortholog thereof, (ii) ATREC8 or an ortholog thereof, and (iii) ATSPO11 or an ortholog thereof. In some embodiments, the MiME expression cassettes comprise at least one expression cassette encoding a gRNA targeting (i) OSD1 or an ortholog thereof, (ii) REC8 or an ortholog thereof, and (iii) PAIR1 or an ortholog thereof. In some embodiments, the plant is a hybrid plant. In some embodiments, the plant is an F1 of a subspecies cross. In some embodiments, the plant is a triploid or tetraploid plant.

### BRIEF DESCRIPTION OF THE DRAWINGS

FIG. 1A-B: Ploidy and genotype of progeny plants of transformation events harboring the T314 and T315 apomixis-inducing T-DNA constructs. 1A: Schematic representation of the T-DNA constructs used to induce the triple MiMe mutation and the triggering of parthenogenesis, resulting in synthetic apomixis. Upper: T313 sgRNA MiMe T-DNA Middle: T314 sgRNA MiMe_pAtECS:BBM1 T-DNA Lower: T315 sgRNA_pOsECS:BBM1 T-DNA: LB and RB: left and right borders of the T-DNA; p35S: promoter of the Cauliflower Mosaic Virus (CaMV); 35S: polyadenylation signal of CaMV; hpt cat int: hygromycin phosphotransferase II with castor bean catalase intron; ZmUbi: promoter, first intron and first exon of the maize Ubiquitin 1 gene; “Os” Cas9: rice-optimized Cas9 coding sequence; NLS: nucleus localization signal fused to Cas9; OSD1, OSD1/2, PAIR1 and REC8: Four cassettes including sgRNAs (20 bp crRNA specific to the target gene+82 bp tracR RNA) driven by the Pol III U3 promoter targeting OsOSD1, PAIR1, and OsREC8; EC1.2: egg cell-specific promoter from Arabidopsis 21,22; OsECA1: egg cell-specific promoter from rice 31,50. 1B: Principle for formation of tetraploid and diploid clonal progenies in MiMe and MiMe+BBM1 plants, respectively. C: Representative flow cytometry histograms of DAPI-marked nuclei suspensions isolated from young leaf blade of a diploid (upper) and tetraploid (lower) progeny plants. D: upper: Genealogy of the plants selected for whole-genome sequencing including 1F and D24 parents (two plants each); heterozygous F1 hybrid BRS-CIRAD 302 (two plants), six F2 sexual progenies; T314 15.1 and 37.7 primary transformants (T0); three T1 progeny plants of each of the sequenced TO plants; three T2 progenies of each of the sequenced T1 plants (i.e., nine T2 plants per event). Lower: Graphical representation of genotypes of the 12 rice chromosomes established from whole genome sequences of homozygous parents, heterozygous F1 hybrid, F2 progeny plants, TO events T314 15.1 and 37.7 and their T1 and T2 progenies. Changes in color along F2 progeny chromosomes mark heterozygous-to-homozygous transitions resulting from meiotic crossovers.

FIG. 2A-F: Phenotype, panicle fertility, and grain quality of progeny plants of selected apomictic events harboring the T314 and T315 T-DNA constructs. 2A: Phenotypes of plants grown under controlled greenhouse conditions: Left: Five F2 progeny plants derived from the self-fertilization of BRS-CIRAD 302 compared to a BRS-CIRAD 302 F1 plant. Right: Three T1 progenies from T314 15.1 event compared to a BRS-CIRAD 302 F1 plant. 2B: Phenotypes of T2 progenies grown under controlled greenhouse conditions: 5-6 T2 progeny plants of a T1 plant of events T314 15.1, T314 37.7, T315 5.4 and T315 8.1 are compared to a BRS-CIRAD 302 F1 plant. Senescent leaves of the plants have been removed for photographing. 2C: Panicles of the BRS-CIRAD 302 F1 hybrid and of T314 15.1 T2 plants. The master panicles of five distinct plants have been pooled for photographing. 2D: Distribution of seed filling rate among BRS-CIRAD 302 F1 plants, and T2 progeny plants of T314 events (15.1 and 37.7) and T315 events (5.4 and 8.1 events). Average panicle fertilities of the apomictic lines range from 60 to 80% of those of the control plants. Significance of the differences are based on Duncan's test. 2E: Husked and dehulled seeds of 1F and D24 parents, F1 and F2 generations and apomictic lines. Upper: 1F and D24 parents, F1 hybrid seeds harvested on 1F parent, F2 seeds harvested on the F1 hybrid. Lower: T3 seeds harvested from apomictic plants in the four selected apomictic lines. 2F: Starch and amylose content of F2 seeds and T3 seeds harvested from BRS-CIRAD 302 F1 plants and T2 apomictic plants, respectively. Different letters indicate significant differences (α-risk=0.05) in a Kruskal-Wallis test: Event T314 37.7 exhibits a significantly lower starch content than Event T315 8.1.

FIG. 3A-B. FIG. 3A. Synthetic apomixis construct used to transform KalingaIII-Kitaake (KKIII) indica-japonica hybrids. This is the same construct that was used for transformation of hybrid BRS-CIRAD 302. FIG. 3B. Photos of panicles from three independent KKIII transformants. There is variation between the events, with the left and middle plants showing high fertility, whereas the plant on the right has fertility similar to that observed with untransformed K-KIII hybrids. The middle plant (line KK32.3), displayed a balance of an acceptable fertility of 87% and a moderate parthenogenesis frequency of 60%. The KK-III hybrid plants with the synthetic apomixis trait were generated using the same protocols as the BRS-CIRAD 302 hybrids described in FIGS. 1, and 2, and detailed in the Methods section of Example 1.

### DEFINITIONS

An “endogenous” or “native” gene or protein sequence, as used with reference to an organism, refers to a gene or protein sequence that is naturally occurring in the genome of the organism.

A polynucleotide or polypeptide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “promoter,” as used herein, refers to a polynucleotide sequence capable of driving transcription of a coding sequence in a cell. Thus, promoters can include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

A “transgene” is used as the term is understood in the art and refers to a heterologous nucleic acid introduced into a cell by human molecular manipulation of the cell's genome (e.g., by molecular transformation). Thus, a “transgenic plant” is a plant that carries a transgene, i.e., is a genetically-modified plant. The transgenic plant can be the initial plant into which the transgene was introduced as well as progeny thereof whose genomes contain the transgene.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read through by a polymerase, and/or formation of double-stranded duplexes, and do not significantly alter expression of a polypeptide encoded by that nucleic acid.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

The terms “identical” or percent “identity,” in the context of two or more nucleic acids or polypeptide sequences, refer to two or more sequences or subsequences that are the same or have a specified percentage of nucleotides or amino acid residues that are the same, when compared and aligned for maximum correspondence over a comparison window, as measured using one of the following sequence comparison algorithms or by manual alignment and visual inspection. Two nucleic acid sequences or polypeptides are said to be “identical” if the sequence of nucleotides or amino acid residues, respectively, in the two sequences is the same when aligned for maximum correspondence as described below. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, California, USA).

The phrase “substantially identical,” used in the context of two nucleic acids or polypeptides, refers to a sequence that has at least 50% sequence identity with a reference sequence (e.g., any of SEQ ID NOs: 1-59). Alternatively, percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein; preferably BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

An “expression cassette” refers to a nucleic acid construct that, when introduced into a host cell, results in transcription and/or translation of an RNA or polypeptide, respectively.

### DETAILED DESCRIPTION OF THE INVENTION

The inventors have discovered that introduction into plant tissue (e.g., embryonic plant tissue or other regenerable plant tissue) of a single nucleic acid construct comprising (i) a first expression cassette comprising an egg-specific promoter operably-linked to a polynucleotide encoding a Babyboom polypeptide, (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype sufficient expression cassettes to induce a meiosis-to-mitosis (Mime) phenotype greatly improves efficiency of generation of plants that produce clonal seed. Moreover, it has been discovered that these methods can be useful for producing hybrid plants that are fertile and produce clonal seed where the hybrids were generated from a cross of two subspecies. Different subspecies can be highly infertile, often able to make F1 plants, but the F1 plants themselves can be highly infertile. As shown herein, by introduction of the expression cassettes as described herein, one can obtain F1 plants that generate clonal seed, not only allowing for high fertility, but also maintenance of heterosis in the plant hybrid in future progeny. Finally, the expression cassettes described herein can also be used to generate triploid or tetraploid or higher ploidy plants that produce fertile clonal progeny.

The single nucleic acid construct described herein comprise an expression cassette for the expression of a Babyboom polypeptide, an RNA-guided nuclease, and sufficient gRNAs to generate the Mime phenotype, and optionally an expression cassette expressing a selectable (e.g., antibiotic or herbicide resistance) marker. Each of these will be described in turn.

Any naturally- or non-naturally-occurring active BABYBOOM polypeptide from a sexually reproducing plant can be expressed as described herein so long as the polypeptide (and/or RNA encoding the polypeptide) is expressed in egg cells in the plant. BABY BOOM polypeptides contain two conserved AP2 domains. The corresponding transcripts lack a miR172 binding site, thereby distinguishing BABY BOOM polypeptides from many other AP2 domain proteins that contain a miR172 binding site. In some embodiments, the BABYBOOM polypeptide is from a species of plant of the genus Abelmoschus, Allium, Apium, Amaranthus, Arachis, Arabidopsis, Asparagus, Atropa, Avena, Benincasa, Beta, Brassica, Cannabis, Capsella, Cica, Cichorium, Citrus, Citrullus, Capsicum, Carthamus, Cocos, Coffea, Cucumis, Cucurbita, Cynasa, Daucus, Diplotaxis, Dioscorea, Elais, Eruca, Foeniculum, Fragaria, Glycine, Gossypium, Helianthus, Heterocallis, Hordeum, Hyoscyamus, Ipomea, Lactuca, Lagenaria, Lepidium, Linum, Lolium, Luffa, Luzula, Lycopersicon, Malus, Manihot, Majorana, Medicago, Momodica, Musa, Nicotiana, Olea, Oryza, Panicum, Pastinaca, Pennisetum, Persea, Petroselinium, Phaseolus, Physalis, Pinus, Pisum, Populus, Pyrus, Prunus, Raphanus, Saccharum, Secale, Senecio, Sesamum, Sinapis, Solanum, Sorghum, Spinacia, Theobroma, Trichosantes, Trigonella, Triticum, Turritis, Valerianelle, Vitis, Vigna, or Zea. In some embodiments the BABYBOOM polypeptide is identical or substantially identical to any of SEQ ID NOs: 1, 2, 3, 4, 5, 6, 7, 8, 9, 10, 11, 12, 13, 14, 15, 16, 17, 18, 19, or 20. See, also, Chahal, et al., Front. Plant Sci., 14 Jul. 2022.

In some embodiments, the plant comprises heterologous expression cassette comprising a promoter that at least directs expression to egg cells operably linked to a BABYBOOM polypeptide as described herein. In some embodiments, the promoter is egg cell-specific, meaning the promoter drives expression only or primarily in egg cells. “Primarily” means that if there is expression in other tissue the levels are no more than 1/10 of the expression levels in egg cells as measured by quantitative RT-PCR.

Exemplary promoters that drive expression in at least egg cells of a plant include, but are not limited to, the promoter of the egg-cell specific gene EC1.1 (e.g., SEQ ID NO: 23), EC1.2, EC1.3, EC1.4, or EC1.5. See, e.g. Sprunck et al. Science, 338:1093-1097 (2012); AT2G21740; Steffen et al., Plant Journal 51:281-292 (2007). In some embodiments, the rice-specific promoter comprises SEQ ID NO:22, i.e., the rice egg cell-specific promoter sequence from the LOC_Os03g18530 OsECA1 gene. In some embodiments, the Arabidopsis DD45 promoter is used to express in rice egg cell (Ohnishi et al. Plant Physiology 165:1533-1543 (2014). An exemplary DD45 promoter sequence can comprise, for example, SEQ ID NO:21. Other promoters that can be used for egg cell expression include promoters of the egg cell-specific ECS1 (SEQ ID NO:60) and ECS2 (SEQ ID NO: 61) genes (Yu et al., 2021 Nature 592:433-437) and the RWD2 gene (Köszegi et al. 2011 The Plant Journal 67:280-291).

Other promoters that are expressed in egg cells, but are not necessarily egg-cell specific, are described in, e.g., Anderson et al., The Plant Journal 76:729-741 (2013). In some embodiments, the expression cassette further comprises a transcriptional terminator. Exemplary terminators can include, but are not limited to, the rbcS E9 or nos terminators. In some embodiments, the expression cassette will include an egg cell enhancer. Exemplary egg cell enhancers include, but are not limited to, the EC1.2 enhancer or EASE enhancer (Yang et al., Plant Physiol. 139:1421-32 (2005).

The single construct will also comprise an expression cassette comprising a promoter operably linked to an RNA-guided nuclease. The RNA-guided nuclease can recognize a sequence of a target nucleic acid (e.g., via an RNA guide), bind to the target nucleic acid, and modify the target nucleic acid. The RNA-guided nuclease has nuclease activity. For example, the RNA-guided nuclease can modify the target nucleic acid by cleaving the target nucleic acid. After the action of the nuclease at the beginning of a coding sequence (as targeted by a gRNA), the introduction of inserts or deletions by the error-prone non-homologous end joining repair of double-strand breaks (DSBs) introduces frame-shift mutations and for example subsequent premature stop codons, leading to mRNA elimination by nonsense-mediated mRNA decay. For example, the Cas nuclease can direct cleavage of one or both strands at a location in a target nucleic acid. Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015: 40 (1): 58-66). Type II Cas nucleases include Cas1, Cas2, Csn2, Cas9, and Cfp1. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470.

Cas nucleases, e.g., Cas9 nucleases, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

Cas9 protein refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 can be a fusion protein, e.g., the two catalytic domains are derived from different bacteria species.

In some embodiments, a Cas protein can be a Cas protein variant. For example, useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the Cas9 nuclease can be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9 (1.0)), and SpCas9 (K848A/K1003A/R1060A) (also referred to as eSpCas9 (1.1)) variants described in Slaymaker et al., Science, 351 (6268): 84-8 (2016), and the SpCas9 variants described in Kleinstiver et al., Nature, 529 (7587): 490-5 (2016) containing one, two, three, or four of the following mutations: N497A, R661A, Q695A, and Q926A (e.g., SpCas9-HF1 contains all four mutations).

The promoter operably linked to the sequence encoding the RNA guided nuclease can be a constitutive promoter or an egg-specific promoter or be otherwise selected such that the RNA guided nuclease is expressed in egg cells. Examples of constitutive promoters include the cauliflower mosaic virus (CaMV) 35S transcription initiation region, the 1′- or 2′-promoter derived from T-DNA of Agrobacterium tumafaciens, the parsley UBI promoter (Kawalleck et al., Plant Mol Biol. (1993 February) 21 (4): 673-84), RPS5 (Hiroki Tsutsui et al. Plant and Cell Physiology (2016)); 2X35SΩ2 (Belhaj, Khaoula, et al. Plant methods 9.1 (2013): 39); AtUBI10 (Callis J, et al. Genetics 139:921-939 (1995)); SIUBI10 (Dahan-Meir, Tal, et al. The Plant Journal (2018)); G10-90 (Ishige, Fumiharu, et al. The Plant Journal 18.4 (1999): 443-448) and other transcription initiation regions from various plant genes known to those of skill. In some embodiments, each expression cassette in the single construct uses a different promoter.

The RNA-guided nuclease will be expressed with a sufficient set of expression cassettes directing expression of guide RNAs (gRNAs) to induce a meiosis-to-mitosis phenotype. Plant genes to be targeted to obtain a MiMe phenotype are known and are also described below. In general, expression of a single guide RNA per gene can be sufficient to reduce expression of each target gene, but if desired, two or more guide RNA can be targeted to one of more of the genes to further reduce its expression.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence. The guide sequence can be used in a single-guide RNA (sgRNA) as described below, or in a split crRNA+tracrRNA construct.

In some embodiments, the targeted nuclease (e.g., a Cas protein) is guided to its target DNA by a single-guide RNA (sgRNA). An sgRNA is a version of the naturally occurring two-piece guide RNA (crRNA and tracrRNA) engineered into a single, continuous sequence. An sgRNA typically contains (1) a guide sequence (e.g., the crRNA equivalent portion of the sgRNA) that targets the Cas protein to the target DNA, and (2) a scaffold sequence that interacts with a nuclease such as a Cas protein (e.g., the tracrRNAs equivalent portion of the sgRNA). An sgRNA may be selected using a software. As a non-limiting example, considerations for selecting an sgRNA can include, e.g., the PAM sequence for the Cas9 protein to be used, and strategies for minimizing off-target modifications. Tools such as NUPACK® and the CRISPR Design Tool can provide sequences for preparing the sgRNA, for assessing target modification efficiency, and/or assessing cleavage at off-target sites.

The guide sequence in the sgRNA may be complementary to a specific sequence within a target DNA. The 3′ end of the target DNA sequence can be followed by a PAM sequence. Approximately 20 nucleotides upstream of the PAM sequence is the target DNA. In general, a Cas9 protein or a variant thereof cleaves about three nucleotides upstream of the PAM sequence. The guide sequence in the sgRNA can be complementary to either strand of the target DNA.

The promoter operably linked to the sequence encoding the guide RNA can be a constitutive promoter or an egg-specific promoter or be otherwise selected such that the guide RNA is expressed in egg cells. Examples of constitutive promoters include the cauliflower mosaic virus (CaMV) 35S transcription initiation region, the 1′- or 2′-promoter derived from T-DNA of Agrobacterium tumafaciens, the parsley UBI promoter (Kawalleck et al., Plant Mol Biol. (1993 February) 21(4): 673-84), RPS5 (Hiroki Tsutsui et al. Plant and Cell Physiology (2016)); 2X35SΩ2 (Belhaj, Khaoula, et al. Plant methods 9.1 (2013): 39); AtUBI10 (Callis J, et al. Genetics 139:921-939 (1995)); SIUBI10 (Dahan-Meir, Tal, et al. The Plant Journal (2018)); G10-90 (Ishige, Fumiharu, et al. The Plant Journal 18.4 (1999): 443-448) and other transcription initiation regions from various plant genes known to those of skill.

Genes necessary to knock out for generation of plants having the MiME phenotype are known. See, e.g., US Patent Publication No. 2012/0042408; US Patent Publication No. 2014/0298507, and PCT Publication No. WO 2012/075195. A plant having the MiMe (mitosis instead of meiosis) genotype is a plant in which a deregulation of meiosis results in a mitotic-like division and in which meiosis is replaced by mitosis. Plants having the MiMe genotype produce functional (e.g., diploid) gametes that are genetically identical to their parent. Exemplary MiMe plants combine phenotypes of (1) no second meiotic division, (2) no recombination and (3) modified chromatid segregation. MiMe plants are exemplified by MiMe-1 plants as described by d'Erfurth, I. et al. PLOS Biol 7, e1000124 (2009) and WO2001/079432) and MiMe-2 plants as described by d'Erfurth, I. et al. PLOS Genet 6, e1000989 (2010). In some embodiments, the MiMe phenotype is induced by inhibiting or mutating OSD1 or an ortholog thereof, REC8 or an ortholog thereof, and at least one of SPO11 or PRD1, or PRD2 or PRD3/PAIR1 (see, e.g., Mieulet D., Cell Res. 2016 November; 26 (11): 1242-1254).

Exemplary MiMe-1 plants combine inactivation of the OSD1 gene, with the inactivation of two or more other genes, one which encodes a protein necessary for efficient meiotic recombination in plants (e.g., SPO11-1, SPO11-2, PRD1, PRD2, or PAIR1), and whose inhibition eliminates recombination and pairing (see, e.g., Grelon M, et al. EMBO Journal 20, 589-600 (2001)), and another which encodes a protein necessary for the monopolar orientation of the kinetochores during meiosis, e.g., REC8, and whose inhibition modifies chromatid segregation (see, e.g., Chelysheva L, et al., Journal of Cell Science 118, 4621-4632. (2005)]. Exemplary MiMe-2 plants combine inactivation of the TAM gene with the inactivation of two or more other genes, one which encodes a protein necessary for efficient meiotic recombination in plants (e.g., SPO11-1, SPO11-2, PRD1, PRD2, or PAIR1), and whose inhibition eliminates recombination and pairing, and another which encodes a protein necessary for the monopolar orientation of the kinetochores during meiosis, e.g., REC8, and whose inhibition modifies chromatid segregation.

Exemplary OSD1 gene sequences include, e.g., those described in US Patent Publication No. 2014/0298507 and rice and Arabidopsis OSD1 protein sequences as provided in SEQ ID NOS: 25 and 27, respectively.

Exemplary TAM gene sequences are described in, e.g., US Patent Publication No. 2014/0298507. Arabidopsis TAMI protein sequence is provided as SEQ ID NO:37. Illustrative rice Cyclin-A1 protein sequences are provided as SEQ ID NOS: 39, 41, 43, 45, and 47. Illustrative Cyclin-A3 protein sequences are provided as SEQ ID NOS: 49 and 51.

Exemplary Arabidopsis DYAD cDNA coding sequence and the sequence of the protein encoded by the nucleic acid are provided as SEQ ID NOS: 70 and 71, respectively. Exemplary rice DYAD homolog (SWITCH1) protein sequences are provide as SEQ ID NOS: 55, 57, and 59.

Examples of SPO11-1 and SPO11-2 proteins are provided in US Patent Publication No. 2014/0298507. An illustrative Arabidopsis SPO11-2 protein sequence is provided as SEQ ID NO:31.

Arabidopsis PAIR1 is described in, e.g., US Patent Publication No. 2014/0298507. An exemplary rice PAIR1 protein sequence is provided as SEQ ID NO:29.

Exemplary rice and Arabidopsis REC8 protein sequences are provided as SEQ ID NOS: 51 and 53, respectively.

In some embodiments, sufficient expression cassettes to produce the MiMe phenotype include at least one expression cassette comprising a promoter operably linked to one or more guide RNA targeting a gene or coding sequence encoding (a) a TAM (Cylin A CYCA1;2) or DYAD protein or ortholog thereof; (b) a protein involved in initiation of meiotic recombination in plants exemplified herein as SPO11-1; SPO11-2; PRD; PRD2; or PAIR1 (also called PRD3) or ortholog thereof; and (c) a protein necessary for the monopolar orientation of the kinetochores during meiosis for example REC8 protein or ortholog thereof. Orthologs have the functionality of the proteins described herein but are from different plant species. Orthologs can be substantially identical to the polypeptides as provide herein or can otherwise be selected from genomic databases.

In some embodiments, sufficient expression cassettes to produce the MiMe phenotype include at least one expression cassette comprising a promoter operably linked to one or more guide RNA targeting a gene or coding sequence encoding (a) an OSD 1 protein or ortholog thereof; (b) a protein involved in initiation of meiotic recombination in plants exemplified herein as SPO11-1; SPO11-2; PRD; PRD2; or PAIR1 (also called PRD3) or ortholog thereof; and (c) a protein necessary for the monopolar orientation of the kinetochores during meiosis, for example REC8 protein or ortholog thereof.

As noted above, the above-described expression cassettes are delivered on a single nucleic acid (e.g., DNA) construct, thereby allowing for highly efficient induction of parthenogenesis. While the methods are not limited to a particular method of transformation, in some embodiments, the single construct is delivered by Agrobacterium as a T-DNA. For example, the single DNA construct compromising the expression cassettes as described herein can be combined with suitable T-DNA flanking regions and introduced into a conventional Agrobacterium tumefaciens host vector. The virulence functions of the Agrobacterium tumefaciens host will direct the transfer of the T-DNA into plant cells when the cell is infected by the bacteria. Agrobacterium tumefaciens-mediated transformation techniques, including disarming and use of binary vectors, are well described in the scientific literature. See, for example, Horsch et al. Science 233:496-498 (1984), and Fraley et al. Proc. Natl. Acad. Sci. USA 80:4803 (1983).

In some embodiments, transformation will be performed on embryonic plant tissue. For example, Agrobacterium tumefaciens can be co-cultivated with seed embryo-derived secondary calluses (see, e.g., Sallaud, C. et al., Theor. Appl. Genet. 106, 1396-1408 (2003); U.S. Pat. No. 10,584,345; EP0290395; and US2011/0212525). In some embodiments, transformation will be performed on somatic tissue. In some embodiments, transformation will be performed on plant protoplasts. In some embodiments, transformation will be performed on immature leaves, inflorescences, pollen or other regenerable tissue. Transformed cells can subsequently be selected (e.g., selecting for antibiotic resistance or other selectable marker introduced with the T-DNA or as otherwise known in the art). Primary transformed cells can subsequently be regenerated into plants.

The plant manipulated as described herein can be any plant species. In some embodiments, the plant is a dicot plant. In some embodiments the plant is a monocot plant. In some embodiments, the plant is a grass. In some embodiments, the plant is a cereal (e.g., including but not limited to Poaceae, e.g., rice, barley, wheat, maize). In some embodiments, the plant is a species of plant of the genus Abelmoschus, Allium, Apium, Amaranthus, Arachis, Arabidopsis, Asparagus, Atropa, Avena, Benincasa, Beta, Brassica, Cannabis, Capsella, Cica, Cichorium, Citrus, Citrullus, Capsicum, Carthamus, Cocos, Coffea, Cucumis, Cucurbita, Cynasa, Daucus, Diplotaxis, Dioscorea, Elais, Eruca, Foeniculum, Fragaria, Glycine, Gossypium, Helianthus, Heterocallis, Hordeum, Hyoscyamus, Ipomea, Lactuca, Lagenaria, Lepidium, Linum, Lolium, Luffa, Luzula, Lycopersicon, Malus, Manihot, Majorana, Medicago, Momodica, Musa, Nicotiana, Olea, Oryza, Panicum, Pastinaca, Pennisetum, Persea, Petroselinium, Phaseolus, Physalis, Pinus, Pisum, Populus, Pyrus, Prunus, Raphanus, Saccharum, Secale, Senecio, Sesamum, Sinapis, Solanum, Sorghum, Spinacia, Theobroma, Trichosantes, Trigonella, Triticum, Turritis, Valerianelle, Vitis, Vigna, or Zea. In some embodiments, the plant is a rice, barley, wheat, maize, sorghum, millet, oat, triticale, rye, fonio, or other cereal plant. In some embodiments, the plant transformed is an F1 embryo is a hybrid resulting from a cross of two in-bred plant lines. For example, plant hybrids are offspring of parents that differ in genetically determined traits. In some embodiments, the F1 embryo is heterozygous, i.e. differing in nucleotide sequence between the parental alleles, in at least 10, 20, 30, 40, 50, 60, 70, 80% or more genes.

Use of a single construct to transform an F1 hybrid results in greatly increased efficiency of parthenogenesis compared to earlier methods of delivery of the relevant nucleic acids. See, e.g., Example 1. For example, in some embodiments, the efficiency of parthenogenesis of plants generated by the methods described herein (i.e., with one construct comprising all of the expression cassettes described) are at least 80%, 85%, 90% or 95%, meaning that at least this percentage of progeny of a plant generated by the methods will be clonal, i.e., genetically the same.

As noted in Example 2, while the methods are applicable to any plant, the methods can be used to generate agronomically viable seed from Fls resulting from crosses of diverse subspecies that would generally result in F1s having low or rare fertility. Exemplary subspecies that can be crossed to form an F1 embryo that is subsequently transformed by the single construct described herein can include, for example, rice subspecies indica and japonica and aus. For example in some embodiments, the F1 is the result of a cross between indica and japonica, aus and japonica, or indica and aus. In some embodiments, the F1 is the result of a cross between Common wheat (Triticum spp.) with rye (Secale cereale L.). This cross produces Triticale, which is a desirable crop due to its general vigor, seed quality and hardiness.

In some embodiments, the cross is between a wild and a domesticated plant. As an example, in rice, Oryza sativa is a cultivated species and its wild relative is Oryza rufipogon. In another embodiments, African cultivated rice is Oryza glaberrima and its wild relative is Oryza barthii. Accordingly in some embodiments, the F1 embryo is from a cross between Oryza sativa and Oryza rufipogon or between Oryza glaberrima and Oryza barthii. In some embodiments, the cross is between two different species that naturally produce infertile or inviable F1 progeny. Examples can include but are not limited to crosses between wheat and rye to make triticale.

The methods described herein can also be used to generate triploid or tetraploid or plants of higher ploidy that will have the capacity to reproduce, by forming clonal seeds. For example, sugarcane hybrids give rise to complex higher polyploids, that currently can only be propagated by cuttings. Because the methods described herein allow a plant to bypass meiosis in seed formation, a cross between a diploid and haploid plant, or two diploids, or a haploid and a triploid, or other ploidy combinations (for example but not limited to odd number ploidy such as 5, 7, 9, etc.) that would normally result in inviable or infertile progeny, can now be used to generate plants of any ploidy that produce clonal seeds of the same ploidy. For example, one can generate triploid embryonic tissue comprising the expression cassettes for egg expression of Babyboom and induce the MiMe phenotype as described herein by crossing a male or female diploid parent comprising expression cassettes for egg expression of Babyboom and induce the MiMe phenotype with a female or male (respectively) haploid parent expressing expression cassettes for egg expression of Babyboom and induce the MiMe phenotype. The diploid parent will produce diploid unreduced gametes and the haploid plant will produce haploid unreduced gametes. Because the endosperm will not develop properly (endosperm requires the genomes of the male and female gametes to have the same ploidy, and here a haploid and a diploid are crossed), the seeds from the cross will abort. However, embryo rescue can be performed from the immature seeds and cultured to form plants, using standard protocols for embryo rescue in plants. The regenerated plants will be triploid. Notably, the triploid plants will comprise the expression cassettes for egg expression of Babyboom and exhibit the MiMe phenotype, meaning that the triploid will produce clonal seed without going through meiosis. Accordingly, triploid plants that produce clonal seed, and comprising the expression cassettes as describe herein, are provided. The methods above can be applied to generate fertile triploid or plants of higher ploidy from any species of plant. In some embodiments, the plant can be for example a tuber crop plant (e.g., potato, cassava, yam, or other tubers), allowing for distribution of the plants by seed rather than as currently occurs (i.e., via tubers).

### EXAMPLES

### Example 1

In the present report, we introduced such an “all-in-one” T-DNA construct into the F1 seed embryo-derived calli of a commercial hybrid of rice. We observed a high frequency of clonal seeds (>80%) in the majority of the triple MiMe mutants and some lines attained a >95% frequency of clonal seeds, which was found to be stable over three generations. The phenotype and the genotype of the apomictic lines were similar to those of the original F1 hybrid and remained stable over generations.

### Results

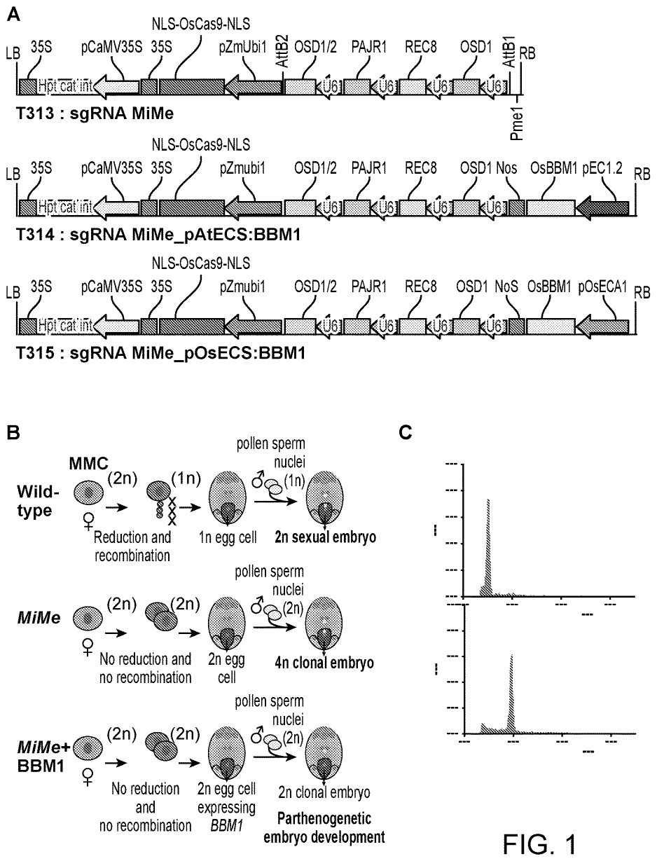

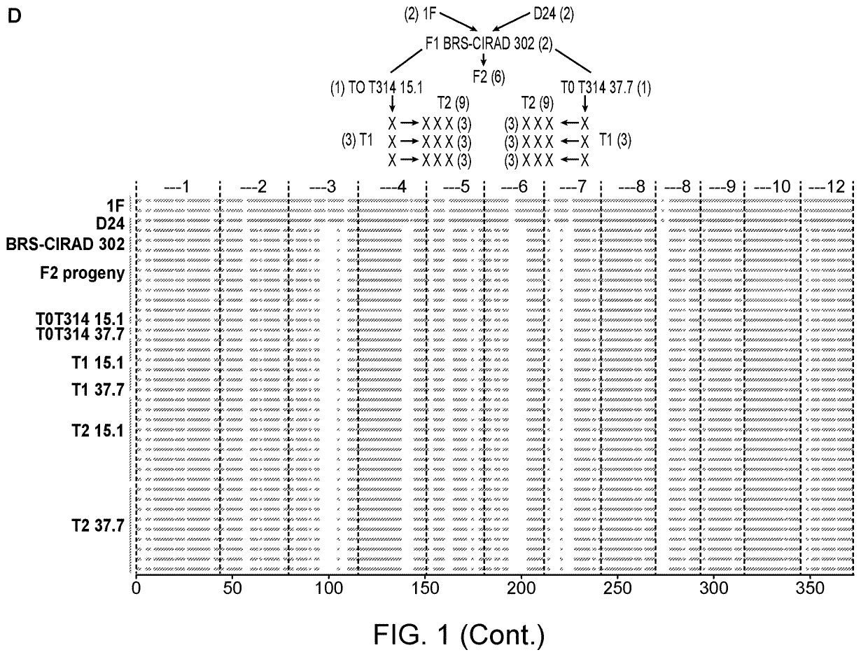

We selected the commercial F1 hybrid BRS-CIRAD 302 rice, which is known for its superior grain quality, to induce apomixis in a single step by combining both the inactivation system creating the triple MiMe mutations and the BBM1 parthenogenesis inducer. Three T-DNA constructs were prepared for introduction into BRS-CIRAD 302 F1 seed embryo-derived callus cells (FIG. 1A): the first construct (sgMiMe=T313) was designed to induce by CRISPR/Cas9 the simultaneous inactivation of PAIR1, REC8, and OSD1, leading to suppression of meiosis (apomeiosis) 9,19. The second construct (sgMiMe_pAtECS:BBM1=T314), created in the T313 background, additionally carries BBM1 driven by the egg cell-specific promoter of the Arabidopsis EC1.2 gene^{21,22}, a cassette triggering parthenogenesis from the egg cell¹⁹. As the level of activity of the Arabidopsis promoter in rice could be a limiting factor, possibly responsible for the as yet incomplete penetrance of synthetic apomixis, the third construct (sgMiMe_pOsECS:BBM1=T315) contains the OsBBM1 gene driven by the egg cell-specific promoter of the rice ortholog (ECA1.1) of the Arabidopsis EC1.2 gene. T314 and T315 were both designed to induce synthetic apomixis (FIG. 1B).

Agrobacterium-mediated transformation of mature F1 seed embryo-derived calli of BRS-CIRAD302 with T313, T314, and T315 T-DNAs generated 41, 49, and 88 confirmed primary (TO) transformants, respectively. The frequency of fertile TO plants was on average 53%, 49%, and 44%, in T313, T314, and T315 TO populations, respectively, with a large variation in harvested seeds ranging from 1 to close to 300. The full sterility of a significant fraction (here 50%) of the TO events was not unexpected since inactivation of only one or two of the three MiMe genes leads to meiosis failure and sterility, with only the single osd1 or the triple MiMe mutants retaining fertility^{9,11,23}

The efficiency of CRISPR/Cas9-mediated mutation was first evaluated by examining lesions at the OSD1 locus in all the TO plants. The frequency of biallelic/homozygous editing was 85%, 82%, and 73% in T313, T314, and T315 TO populations, respectively. There was no striking difference in the number of T1 seeds harvested from plants not edited at the OSD1 locus and those with frameshift lesions at this locus. We then examined the mutations at PAIR1 and REC8 in TO plants with more than 10 T1 seeds, which revealed that the vast majority of the events were either wild-type or harboring mutations at the two alleles of the three MiMe target loci. We identified 4, 10, and 18 MiMe mutants harboring triple frameshift biallelic/homozygous mutations in PAIR1, REC8, and OSD1 in the examined T313, T314, and T315 fertile TO plant populations. MiMe-only mutants should produce tetraploid progeny plants resulting from the fusion of male and female diploid gametes (FIG. 1B). Indeed, flow cytometry analysis showed that all the progeny plants of the four selected sgMiMe (T313) events were tetraploid (n=109). (Table 1 and FIG. 1C). In contrast, if parthenogenesis is triggered, a fraction of the T1 progeny should be diploid as previously shown at a frequency of 10-30% 19 (FIG. 1B). Surprisingly, 9 of the 10 sgMiMe_pAtECS:BBM1 (T314) and 13 of the 18 sgMiMe_pOsECS:BBM1 (T315) independent transformants tested produced more than 80% diploid progeny plants-several of them at a rate of more than 97%-suggesting a very high frequency of parthenogenesis (Table 1). The small number of other transformants produced either exclusively tetraploids or a lower frequency of diploids, suggesting a null or lower expression of the BBM1 transgene in these events. Ploidy level as determined by flow cytometry was systematically confirmed by phenotypic observation which allows easy discrimination of diagnostic traits of tetraploid plants (e.g., awned and large spikelets, darker and thicker leaves, lower tillering) from those of diploid plants. Altogether, these results indicate an unexpectedly high frequency of induction of parthenogenetic plants from the egg cell in TO T314 and T315 MiMe events.

To determine whether this high frequency remains stable across generations, we selected two T314 (15.1 and 37.7) and four T315 (3.2, 5.4, 8.1, and 8.2) events, which produced diploid progeny (T1) at a rate of more than 92%. Determination of ploidy level in T2 progeny plants (n>40) of five T1 plants per line showed a very high frequency of diploids, consistent with those observed in TO (Table 2). We then analyzed T3 progeny plants (n=100) from three T2 plants in two T314 (15.1 and 37.7) and two T315 (5.4 and 8.1) events. The germination frequency of T3 seeds was close to 100% in all four events. The four events exhibited average diploid plant formation frequencies that were higher than 90% with events T314 15.1 and T315 5.4 exhibiting frequencies close to 99% and 97%, respectively (Table 2). The high frequency of diploid plants along several generations suggests that the combination of MiMe with egg cell expression of BBM1 triggers clonal reproduction through seeds with high penetrance.

Clonal reproduction is expected to maintain the heterozygosity of the F1 hybrid across generations. To determine if heterozygosity was maintained in T1 and T2 progeny plants, we examined the segregation of four diagnostic markers, which are polymorphic between 1F and D24, the two parents of BRS-CIRAD 302, and distributed on different chromosomes. These four markers segregated independently in an F2 population, as expected. In contrast, the diploid T1 and T2 progenies of all the T314 and T315 events (n=765 T1s and n=880 T2s) exhibited the heterozygous genotype of the F1 hybrid at all four loci, strongly suggesting that they are clones. To test further whether clonal reproduction occurred, we carried out short-read, whole-genome sequencing of the 1F and D24 parents, BRS-CIRAD 302 F1, 6 F2 progenies, 2 TO (events 15.1 and 37.7), and 6 T1 and 18 T2 progenies (FIG. 1D). As expected, the F2 plants exhibited recombined genotypes showing heterozygous-to-homozygous transitions at cross-over positions. In contrast, all the TO, T1, and T2 plants retained the heterozygous parental genotype in the entire genome, showing that they are completely clonal. Altogether, this shows that synthetic apomixis, induced by combining MiMe mutations and egg cell expression of BBM1, allows very efficient clonal propagation of F1 hybrids through seeds.

To test whether the high frequency of apomixis achieved in the BRS-CIRAD 302 hybrid is a consequence of the hybrid genetic background or a function of the design of the “all-in-one” construct, we also transformed our all-in-one T314 and T315 constructs into the inbred Kitkaake cultivar. In contrast to our previous observation of ~30% apomixis frequency with the pAtECS:BBM1 construct¹⁹, up to ~84% of progeny from TO transgenics raised with the T314 construct in Kitaake were found to be apomictic diploids. A similarly high frequency of apomictic T1 progenies was also observed with T315 (rice egg cell promoter fusion) as well. In contrast, the rice egg cell promoter fusion pOsECS:BBM1 by itself, in the absence of the MiMe cassette, did not yield any parthenogenetic progeny (0% haploid out of at least 1,200 T1 plants from 20 independent TO transformants). Thus, it is clear from these results that the construct architecture, rather than genotype or number of transformants generated, is responsible for high-frequency apomixis with the all-in-one constructs.

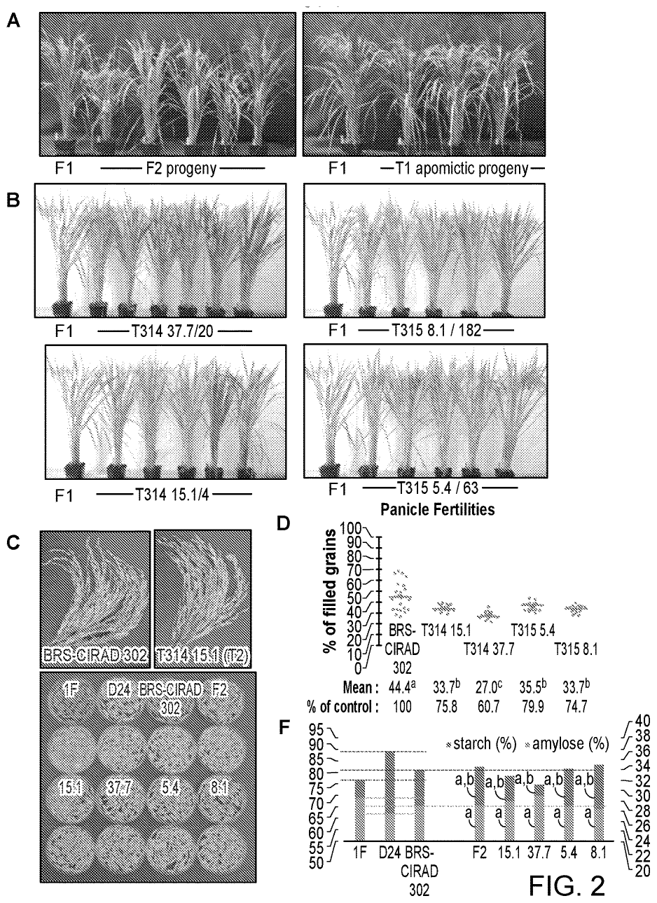

We next explored if clonal apomicts maintain the F1 hybrid phenotype. We first examined the gross phenotype of T1 clonal plants grown along F1 hybrid and sexual F2 progenies. Whereas F2 progenies exhibited obvious disjunction for traits such as heading date, plant height, plant habit, and seed coat color traits, the clonal progenies displayed a uniform phenotype for these traits and flowered synchronously with the F1 hybrid plants (FIG. 2A). Phenotypes were more thoroughly examined in T2 progenies of five T1 plants in two T314 (15.1 and 37.7) and two T315 (5.4 and 8.1) events, grown with control BRS-CIRAD 302 plants (FIG. 2B). No significant difference in plant traits was observed across the five T2 progeny lines derived each from a T1 plant in a given event. Furthermore, the apomictic lines were similar to the control F1 hybrid for most traits (Table 3). The few exceptions comprised a significantly higher tiller number in the T314 37.7 event and shorter flag leaf length and narrower antepenultimate leaf width in two lines (Table 3). Of note, apomictic lines exhibited significant differences between them: T315 5.4 plants were significantly taller than T314 15.1 and 8.1 plants, and T315 8.1 plants exhibited significantly longer panicles than T314 37.7 plants. These differences could be due to somaclonal variation, which is commonly observed in tissue culture-derived rice plants²⁴. In a clonal mode of reproduction, such variations are likely to be fixed. Altogether, phenotypic evaluation of progenies of T314 and T315 events shows that they exhibit a generally uniform phenotype recapitulating that of the F1 hybrid.

As incomplete penetrance of MiMe and/or failure in parthenogenetic embryo development affect fertility, a major trait to examine in apomictic lines was grain filling. We observed a rather large range of variation (20-65% filled seeds, mean=44.4%) in grain filling among the BRS-CIRAD 302 control plants grown in our greenhouse conditions (Table 3 and FIGS. 2C and D). It is not uncommon that lines with an indica genetic background are less fertile than japonica cultivars under greenhouse conditions. As hybrid plants carry the WA cytoplasmic male sterility (cms)²⁵, we examined pollen viability in BRS-CIRAD 302, which is expected to be close to 100% due to the sporophytic mode of restoration of WA26,27. In our greenhouse conditions, BRS-CIRAD 302 anthers exhibited average pollen viability of 60%. Nevertheless, pollen viability that is reduced by half is considered sufficient for full panicle fertilization in rice, as illustrated in commercial F1 hybrids carrying the rice Hong Liang (HL) and Boro II (BT) cms systems, which have a gametophytic mode of male fertility restoration². As for the T2 clonal progenies of T314 and T315 events, they also showed reduced panicle fertilities, with average grain filling rates falling within a narrow 27%-35.5% range (Table 3; FIGS. 2C and D). Thus, although fertilities were reduced in both BRS-CIRAD 302 control plants and apomictic progenies in greenhouse conditions, apomicts showed further reductions in fertility compared to the BRS-CIRAD 302 control plants (Table 3).

To determine the possible causes of this reduced fertility we first examined pollen formation and viability in apomictic lines: as anticipated in MiMe mutants of rice, dyads were formed instead of tetrads. Mature pollen viability reached 80-90%, demonstrating that pollen viability is unlikely to be the cause of the incomplete filling. As incomplete osd1 mutation penetrance in Arabidopsis female meiosis results in entry into the second meiotic division, causing a reduction in female fertility, we next examined the products of female meiosis in two apomictic lines (T314 15.1 and T314 37.7). In both events, a majority of ovules formed dyads (as verified by clearing and callose deposition patterns on meiotic cell walls), followed by degradation of the micropylar spore and selection of the chalazal functional megaspore, resulting in gametogenesis. A range of low-frequency abnormalities were also observed, such as tetrads, triads, and abortive or persisting dyads, which might result in abnormal gametogenesis that could partly explain the reduced female fertility. In addition, imbalance in chromosome segregation may occur at the first division leading to unbalanced 2n spores/gametophytes with reduced viability.

As we also produced T314 and T315 Kitaake plants with high-frequency (84%) apomixis, we next examined the seed set of T1 plants from these lines. While wild-type Kitaake plants exhibited nearly 90% fertility, panicle fertility of apomictic lines reached 74% in a small number of transgenic lines examined. Thus, although apomicts exhibited roughly 16% less fertility compared to wild type, events with high-frequency apomixis and reasonably high (but not yet complete) fertility can be generated in a different genetic background.

MiMe endosperm is expected to be initially hexaploid instead of triploid as it results from the fusion of the two diploid central cell nuclei and a diploid pollen sperm nucleus. To determine whether this may have an impact on grain quality in apomictic seeds, we examined the ploidy level of milky endosperm cell nuclei in developing seeds. As expected, the initial ploidy of control endosperm cells was 3n, whereas in MiMe-derived endosperm this initial 3n peak was replaced by a 6n peak. We also observed 6C and 12C peaks in the control endosperm, thereby confirming the well-reported phenomenon of endoreduplication occurring during the development of the cereal endosperm, which tolerates a mainly 12C-24C level at seed maturity^{28,29}. Because of this active natural endoreplication operating in the endosperm, we had anticipated that there would not be a major impact of the initial ploidy shift on grain quality.

Seed shape and size are important traits contributing to grain quality and end-user acceptance. We, therefore, investigated the variation for these traits in apomictic lines with regard to grains harvested from the F1 hybrid (FIG. 2E): In sexual reproduction, the F2 grain shape is determined by the spikelet shape of the mother F1 plant. If reproduction is clonal, then this initial shape should be preserved along generations. No significant differences were observed between F2 and T3 seeds, with the exception of a lower grain width found in line 37.7. The thousand-grain weights of apomictic lines were not significantly different from those of F2 seeds harvested from F1 hybrid plants, either (Table 3). We then analyzed the starch and amylose contents of T3 seeds of two T314 and two T315 events compared to those of F2 seeds collected from F1 hybrid plants, grown under the same conditions as the apomictic events (FIG. 2F). The embryo and endosperm of seeds borne on the F1 hybrid have a segregating, F2 genotype whereas those of apomictic seeds share the unique F1 genotype. BRS-CIRAD 302 F1 seeds exhibit intermediary starch and amylose contents when compared to the 1F and D24 parental lines. The F2 seeds harvested on F1 hybrid plants exhibited starch and amylose contents similar to those of F1 seeds. No significant difference was observed for starch and amylose contents between the sexual F2 and clonal T3 seeds of the four events. However, line 37.7 exhibited significantly lower starch content than line 8.1. Taken together, these results indicate that endosperms of clonal seeds harvested on an apomictic hybrid plant display overall identical shape and unaltered functional quality traits compared to sexual F2 seeds harvested from the F1 hybrid.

### Discussion

We report here that high-frequency (>95% over three generations) synthetic apomixis can be achieved in hybrid rice: A previous report using the same strategy, but in a two-step procedure, for inducing apomixis in the inbred cultivar Kitaake, reached a maximum efficiency of 29% 19. We have shown here that high-frequency apomictic events can also be generated in cv Kitaake, with a limited transformation effort. This indicates that the difference in efficiency arises from the new construct architecture rather than from the number of analyzed transformation events or the use of a specific genetic background. Whole-genome sequencing of T0, T1, and T2 generations confirmed a faithful transmission of the heterozygous genome through the synthetic apomixis mode of reproduction. Our study establishes for the first time that there is no intrinsic barrier preventing a very high level of synthetic apomixis in a crop species, stable over generations, which is a requisite for application in agriculture.

A related and more specific conclusion is that the requirement for a fertilized endosperm for viable seeds does not set an upper limit for the frequency of parthenogenesis. The necessity for an endosperm could have prevented high-efficiency clonal seed formation because of a potential conflict between parthenogenetic zygote formation and central cell fertilization that triggers endosperm development. The fertilization of the central cell requires the Egg Cell 1 (EC1) protein, which is secreted exclusively by the egg cell and necessary for inducing the two pollen-delivered sperm cells to fuse with both the egg cell and the central cell³⁰. Since EC1 expression becomes undetectable in the zygote^{22,31}, efficient initiation of parthenogenesis could lead to frequent failure of central cell fertilization and jeopardize the formation of viable seeds, setting an upper limit on apomixis frequency. Possible explanations for the very high apomixis efficiency (90-95%) obtained here are that sufficient EC1 protein persists even after initiation of parthenogenesis to activate the sperm cell for fusion with the central cell, or alternately, that the central cell—but not the egg cell—is fertilized before the egg cell develops into an embryo. In any case, our results eliminate a major concern regarding the practical applicability of this system. Mutants with the development of endosperm in the absence of fertilization have been described in Arabidopsis and rice^{32,33}, but these endosperms abort due to the deviation from the essential 1:2 paternal to maternal genomes ratio³². As shown in this study, agronomically useful efficiencies of apomixis can be achieved without incorporating autonomous endosperm development, simplifying its implementation in crop plants.

We also demonstrate that synthetic apomixis allows faithful reproduction of the F1 phenotype in apomictic lines. We observed no major differences in phenological, morphological, and grain quality traits between apomictic lines and control F1 hybrid plants, although some lines exhibited slight differences that can be likely ascribed to a somaclonal source of variation that was fixed through the apomictic mode of reproduction. Generating several apomictic lines may allow an easy selection of lines that are essentially identical to the control hybrid, as illustrated by line T314 15.1 in our study. The genetic basis of heterosis has been intensely studied, and epigenetic mechanisms have been proposed as one of the important contributions to hybrid vigor^{34,35}. A study in Hieracium, which is a natural apomict, used recessive apomixis-deficient mutants to make apomictic hybrids³⁶. These hybrids retained hybrid vigor after asexual propagation, showing that heterosis is heritable through asexual propagation. However, as Hieracium is a facultative apomictic species, its epigenetic control of gene expression can be expected to be adapted for asexual reproduction. In contrast, cultivated Asian rice has reproduced sexually since its domestication ~10 kya, and likely since the origin of the genus Oryza~10 Mya. Studies in the obligate sexual species Arabidopsis have reported that hybrids are sensitive to the epigenetic state of the parents, including parent-of-origin effects^{34,37,38}. Given the evidence for parental epigenetic contributions to heterosis, the absence of any paternal genome contributions in the clonal propagation of hybrids through parthenogenesis might lead to the loss of heterosis. Nevertheless, our observations that hybrid traits are retained for two consecutive generations of clones without obvious negative consequences suggest that heterosis might be predominantly genetically controlled, and demonstrate that epigenetic factors do not restrict the utility of synthetic apomixis for rice hybrids, and possibly for other sexually reproducing crop plants of agricultural significance. Trueness-to-type and hybrid vigor should ultimately be evaluated under a field trial to confirm greenhouse findings.

The BRS-CIRAD 302 control plants exhibited incomplete and highly variable panicle fertility that ranged from 25 to 65% under greenhouse conditions. Pollen viability in the hybrid, which was on average 60%, can probably not explain the defective seed setting rate in the F1 hybrid plants. Also, in the apomictic lines, higher pollen viability (80-90%) was observed without being associated with an improved seed set. A tentative cause of the reduced panicle fertility of the hybrid could be a detrimental interaction of the WA cytoplasm with the greenhouse environment through pleiotropic effects affecting other traits involved in fertilization. The apomictic lines were not fully fertile either under our greenhouse conditions and exhibited an average grain filling rate ranging from 27 to 35% across the lines, representing 60-80% of that of the control F1 hybrid. To determine whether the observed variation can be ascribed to interactions between the genotype and the greenhouse environment, fertility will need to be re-examined in apomictic plants generated from manually produced F1 seeds of hybrid combinations known to exhibit full panicle fertility under greenhouse conditions. The identification of Kitaake T314 and T315 events with both high-frequency apomixis and reasonably high fertility is very encouraging in this respect.

However, as fertility seems to be consistently and significantly reduced in the apomictic lines as compared to control untransformed plants, an alternative/complementary explanation could be an incomplete penetrance of MiMe in female meiosis. Indeed, in both rice and Arabidopsis osd1 mutants, a proportion (10-15%) of the female meiocytes still enter meiosis II,^{9,13}, which is catastrophic in the pair1 rec8 context as massive chromosome mis-segregation occurs at meiosis II. We observed here that the majority of products of female meiosis in apomictic rice lines were dyads, although tetrads and triads also occurred at low frequency, suggesting that entry into meiosis II may occur occasionally, as has been found in Arabidopsis¹³¹³ and rice⁹ osd1. If incomplete penetrance of osd1 reduces the fertility of the apomictic lines, a more robust prevention of meiosis II entry may be obtained by combining manipulation of OSD1 with an additional meiotic cell cycle regulator such as TAM³⁹ or TDM⁴⁰. We also cannot exclude that post-meiotic defects, such as developmental failure of the embryo or the endosperm, also contribute to reduced fertility. The fertility and stability of synthetic apomixis will ultimately have to be tested under a range of environmental situations before a wide application in agriculture.

Parthenogenesis by the expression of BBM-related genes from apomicts was previously demonstrated in multiple cereals^{41,42}. The possibility that zygote-expressed BBM genes from sexual plants can be similarly utilized to bypass fertilization has been proposed³¹ and subsequently demonstrated¹⁹. The MiMe system to make unreduced gametes was originally developed in Arabidopsis⁸. Thus, the results from this study could have wider applicability to efforts to engineer apomixis in other crop plants. As previously discussed by several authors^{4,5,7}, engineered synthetic apomixis can provide a low-cost, immortalized source of F1 seeds that will allow their use by smallholder farmers. In addition, synthetic apomixis holds several specific advantages compared to male sterility-based F1 hybrid seed production. The first is a potential improvement in grain quality, which has long been a limiting factor in F1 hybrids; this has restricted their adoption and has been partly ascribed to the segregating F2 genotype of harvested seed endosperms. In synthetic apomicts, all the endosperms share the same F1 genotype, and this should result in more predictable grain quality features. A second advantage is avoiding the use of a single cytoplasm (e.g., WA cytoplasm) over large acreages of cultivation, which may make F1 hybrids more susceptible to disease outbreak⁴³ or reliant on very specific environments for environmental male sterility seed production systems. A third advantage is that it could widen the breadth of tested hybrid combinations that have so far been restricted by the long and tedious preliminary process of converting the parental lines to cms prior to multisite evaluation. Progress in understanding and harnessing the dispensable genome, which has been demonstrated to harbor a wealth of adaptation genes⁴⁴, may allow a more informed choice of genome combinations for developing future climate-smart apomictic hybrids. Having an efficient tool for converting hybrids to apomixis will be very valuable in this respect. Beyond the well reported yield performance and stability qualities, apomixis should therefore allow breeders to harness the potential of F1 hybrids that exhibit biotic and abiotic stress tolerance and are thus better equipped to deal with the challenges posed by global climate change and increased food demand.

### Example 2

Hybrids are agriculturally important because they can greatly outperform standard varieties through a phenomenon called heterosis or hybrid vigour. In rice, hybrids have been used to increase yields by 25% to 50%. There are two subspecies of rice, japonica and indica. The hybrids currently used are within the subspecies, obtained by crossing two indica parents or two japonica parents. Hybrids of crosses between the subspecies indica and japonica display much greater heterosis than hybrids within each subspecies. Furthermore, under low nitrogen indica/japonica hybrid rice has significantly higher yields than japonica/japonica hybrid rice (Chu et al. 2019, Field Crops Research, Volume 243, 107625, ISSN 0378-4290). However, these inter-subspecies hybrids also show extensive infertility and therefore are not agriculturally useful. Over the years, many efforts have gone into overcoming this fertility barrier through genetic mutants, etc., but with limited success (e.g., Guo et al. 2016 Scientific Reports 6:26878 and Zhang, Journal of Integrative Agriculture Volume 19, Issue 1, January 2020, Pages 1-10). This fertility barrier arises from a phenomenon called pre-zygotic reproductive isolation. In the case of indica/japonica hybrids, it appears to be due to silencing of the japonica allele of gametophyte development genes in the germline. Another source of sterility is the creation of deleterious combination of alleles due to meiotic recombination between the two parental genomes. If advanced progeny lines do recover their fertility, they generally exhibit the parental phenotypes and have lost hybrid vigour. Consequently, the use of japonica/indica hybrids in breeding has been hampered by a lack of useful progeny plants combining favorable traits from the two parents. By allowing the generation of gametes containing both parental allele complements and avoiding meiotic recombination, apomixis has the potential to fully harness the potential of japonica/indica hybrids by circumventing the hybrid sterility and breakdown issues.

It has been discovered that this reproductive isolation barrier can be overcome by using a methods described herein, resulting in synthetic apomixis. A method in rice, called synthetic apomixis, that results in clonal seed formation at frequencies up to 29% (Khanday et al. 2019 Nature) has been described previously. This technique can be used for stable propagation of hybrids without genetic segregation. It involves substitution of mitosis for meiosis in the germline (MiMe), followed by parthenogenesis induced by expressing a transcription factor BBM1 in the egg cell. As discussed in Example 1, improved apomixis vectors for transforming rice hybrids that combine MiMe+BBM1 within a single DNA molecule construct results in high frequencies of apomixis in hybrid rice, resulting in as much as 90-95% clonal progeny.

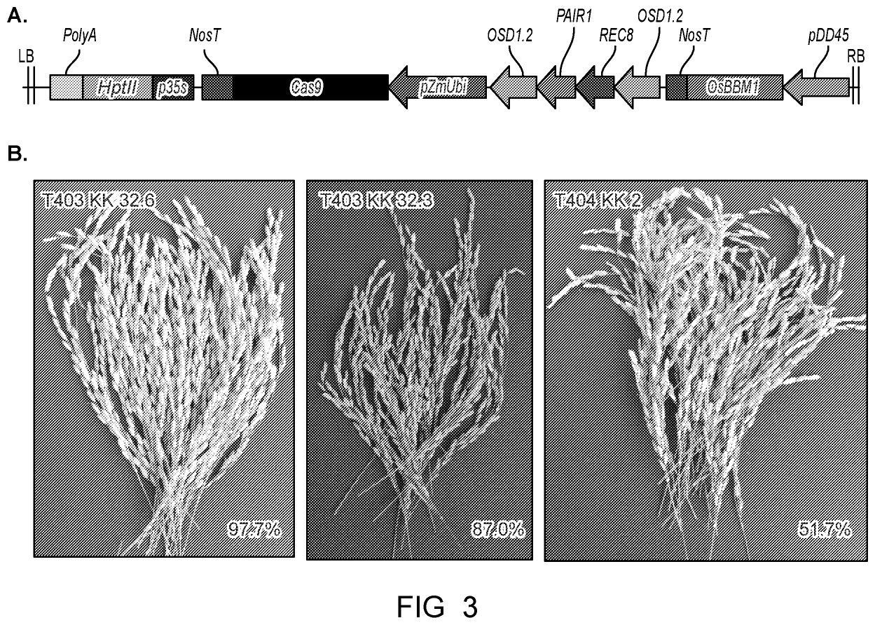

Inter sub-species crosses of japonica cv. Kitaake with indica cv. Kalinga III result in vigorous hybrids but they have low fertility (~50%) preventing their use in agriculture. Transformation of KKIII (Kitaake-KalingaIII) hybrids, i.e., embryonic tissue from hybrids, with the MiMe+BBM1 apomixis construct of Example 1 was performed. See, FIG. 3A. Surprisingly, it was observed that many of the KKIII transformants have significantly elevated fertility, ranging from 87% to 97% in the five best transformants. See, FIG. 3B. This fertility range is in the same range as that of typical intra-subspecies hybrids, as well as for non-hybrid rice. This restoration of fertility can be attributed to the abolition of meiosis and formation of diploid gametes, which will bypass the reproductive isolation between japonica and indica. In addition, most of the KKIII hybrids produced viable clonal diploid progeny through parthenogenesis, at frequencies ranging from 4% to 100%. At least one line KK32.3 displayed a balance of an acceptable fertility of 87% and moderate parthenogenesis frequency of 60%. It is expected that with more events transformants that have higher parthenogenesis frequencies in addition to being fertile can be obtained by these same methods. These results demonstrate that KKIII hybrids (as well as other subspecies hybrids) can be propagated through seeds while retaining agriculturally acceptable fertility.

This method can be applied to inter-subspecies hybrids arising from different combinations of hundreds of indica and japonica genotypes, greatly expanding the availability of hybrid rice by exploiting a new landscape of genetic diversity and heterosis that is currently inaccessible. The method can also applied to other crop plants, in situations where desirable hybrid combinations cannot be cultivated because of sterility arising from pre-zygotic reproductive isolation. Viable zygotes resulting from intergeneric and interspecific hybridization in plants that carry parental chromosomes from both species will produce infertile flowers due to abnormal segregation of parental chromosomes at meiosis (e.g., crosses between wheat and rye, used for making triticale). Skipping meiosis of the intergeneric/interspecific hybrid through apomixis should allow novel combinations of genomes, and propagate novel genome associations through seeds.

The method can also be used to propagate other examples of high performing genetic combinations that have vigorous vegetative growth but poor fertility. For example, triploid plants are more vigorous than diploid plants, but they have high sterility due to failure of gamete formation after meiosis. Because the method eliminates meiosis, viable gametes can be formed even from triploids. This is followed by parthenogenesis using the synthetic apomixis construct, ensuring that the progeny will also be triploid. This step is necessary to avoid the doubling the chromosome number in every generation, that would otherwise occur with sexual embryos after the elimination of meiosis.

Although the prospect of apomixis in crop plants has been rightly hailed as a method of maintaining known commercial hybrids, it has not been previously considered as a method for rescuing vigorous but infertile hybrids. We demonstrate this in a specific hybrid combination in rice. More generally, other barriers to successful reproduction from high performing combinations of genomes, including from odd numbers of genomes such as triploidy, can also be overcome with this new strategy.

### Methods

Plant material and growth conditions: Commercial F1 seeds of BRS-CIRAD 302 (a CIRAD-EMBRAPA hybrid of rice released in 2010 in Brazil), were used in this study. BRS-CIRAD 302 is a high-yielding indica/indica F1 hybrid resulting from crossing a male sterile line of 1F (CIRAD 464), bearing the Wild Abortive (WA) cytoplasmic male sterility, and the D24 line carrying the restorer nuclear genes. Grains harvested on BRS-CIRAD 302 exhibit superior quality, according to Graham's⁴⁵ classification, i.e., a long and slender shape, low breakage rate, and high amylose content. For ascertaining purity in hybrid seed production, 1F harbors a recessive, brown-colored seed coat trait while D24 harbors a dominant, straw-colored seed coat trait. Seed husks borne by the F1 hybrid are therefore straw in color. Transgenic plants (TO, T1, T2, and T3 generations), parental lines 1F and D24, BRS-CIRAD 302 hybrid, and F2 progenies were grown in containment greenhouse facilities under natural light supplemented by light provided by LEDs (12h: 12 h photoperiod) under 60% hygrometry and 28 °C day and 20 °C night temperatures. Wild-type and transgenic events (T0 and T1 plants) of cv. Kitaake were grown at UC DAVIS under greenhouse conditions as previously described¹⁹.

T-DNA construct preparation: Three constructs were prepared (FIG. 1A): 1. the 4sgMiMe (T313) construct was created by inserting a 2,067 bp attB1-Attb2 fragment from the T-DNA of the pCAMBIA2300-MiMe 4sgRs vector¹⁹, containing four single guide RNA (sgRNA) cassettes each driven by an OsU6 promoter⁴⁶, into a pDONR207 vector by Gateway (Clontech) cloning. Two guide RNAs (GAGAAATTCCGGCGGTAGGG and GCGCTCGCCGACCCCTCGGG) were used for OSD1 and one each for PAIR1 (TCGACGACAACCTCCTCACC) and REC8 (GTGTGGCGATCGTGTACGAG). The fragment was then transferred into a pENTRY vector and eventually into the pCAMBIA 1300-based (www.cambia.org) 2G9 binary plasmid through an LR reaction. The 2G9 binary vector T-DNA region harbors a rice codon-optimized Cas9 coding region⁴⁷ driven by the maize ubiquitin promoter⁴⁸, a selectable cassette containing the hpt gene with a catalase intron⁴⁹, driven by the CaMV 35S promoter, and a cmR ccdb Gateway cloning site. 2. The sgMiMe_pAtECS:BBM1 (T314) vector was prepared following blunt-end insertion of the 3,617 bp EcoRI fragment of pCAMBIA1300-DD45: BBM1: Nos¹⁹ containing the Arabidopsis EC1.2 egg cell-specific promoter driving the OsBABYBOOM1 (OsBBM1) coding sequence terminated by the Nopaline synthase (Nos) polyadenylation signal, into the compatible PmeI site of T313, situated proximal to the right border of the T-DNA. 3. The sgMiMe_pOsECS:BBM1 (T315) vector was prepared by releasing the 2,828 bp XbaI-AseI fragment of the pCAMBIA1300_pOsECA1.1: OsBBM1: nosT plasmid, containing the promoter of the rice egg cell-specific OsECA1 (LOC_Os03g18530) gene^{31,50} driving OsBBM1 terminated by the Nos polyadenylation signal (Khanday and Sundaresan, unpublished), followed by its blunt-end insertion into the PmeI cloning site of T313. The inward orientation of the egg cell-specific promoter: OsBBM1 cassettes adjacent to the T-DNA RB was ascertained in both T314 and T315 by sequencing. The sequences of the sgRNAs targeting rice PAIR1, REC8 and OSD1, the BBM1 coding region, and the Arabidopsis thaliana EC1.2 promoter region sequences were those originally detailed in refs.^{21,30}. The binary vectors were introduced into Agrobacterium tumefaciens strain EHA105 by electroporation. The three constructs were cloned by the GENSCRIPT company (Leiden, the Netherlands).

Plant transformation: Transformation of BRS-CIRAD 302 was carried out by co-cultivation of mature F1 seed embryo-derived secondary calluses with EHA105 Agrobacterium cell suspensions, selection of transformed cell lines based on hygromycin tolerance, and primary transformant (TO) regeneration following the procedure detailed by Sallaud and co-workers⁵¹. Slight changes to the procedure include the reduction of Agrobacterium cell suspension OD to 0.01 and lengthening of some phases of selection due to slower growth of transformed indica cell lines compared to those of standard japonica cultivars. Two rounds of transformation were carried out for the T314 and T315 vectors. Transformation efficiencies using the T313, T314, and T315 vectors in BRS-CIRAD 302 were determined. Eventually, 41, 49, and 88 primary transformants of the 3 respective populations were transferred to the containment greenhouse and grown until the harvesting of T1 seeds. The primary transformants are numbered after both the co-cultivated callus number (e.g., callus 21) and the hygromycin-resistant cell line number (e.g., hygromycin-resistant cell lines 21.1, 21.2 and 21.3) they originate from. The several hygromycin-resistant cell lines deriving from a single co-cultivated callus are generally independent transformation events⁵¹. Twenty primary transformants for each of the T314 and T315 T-DNA vectors were raised in the Kitaake cultivar using Agrobacterium-mediated transformation with the EHA105 strain.

Molecular characterization of primary transformants and progeny: DNA from primary transformants and T1 and T2 progeny, as well as of control plants, was isolated using MATAB and automated procedures in 96-well dishes. Quantification of T-DNA copy number in primary transformants was achieved by Q-PCR analysis of the hpt gene copies according to ref.⁵². The presence of both Cas9 and of the egg-cell specific cassettes was ascertained by PCR in TO transformants selected for analysis. The efficiency of the sgRNA at generating mutations at their respective target sites was evaluated through PCR and sequencing, using primers amplifying their target regions in the two alleles of BRS-CIRAD 302. For OSD1, natural polymorphisms existing in the two parental sequences compared to the Osd1-1 crRNA target sequence, originally designed for the japonica cv. Kitaake, made the Osd1-1 sgRNA inefficient at inducing lesions at this site. A second target site in OsOSD1 was originally added because of the rather low efficiency of Osd1-1 at creating lesions in cv. Kitaake (Khanday and Sundaresan, unpublished). All the T1 and T2 progeny plants for which ploidy was determined were also analyzed, along with parental and hybrid and F2 control plants, for segregation of four polymorphic microsatellites markers located in the middle of the long arms of chromosomes 1 (RM1), 8 (RM25), 9 (RM215) and 11 (RM287). The F2 progenies (n=12) were analyzed to check for the free segregation of the parental alleles. The SSR work was carried out at the genotyping facility of Cirad in Montpellier (France). Whole-genome sequencing of 1F, D24, BRS CIRAD 302 (2 plants each), 6 F2 plants, 2 TO plants (T314 15.1 and 37.7), 6 T1 plants and 18 T2 plants was carried out on the Illumina sequencing platform of the MPIPZ in Cologne (Germany). The genealogy of the materials used for sequencing is summarized in FIG. 1D.

Whole-genome sequencing and genotype calling: The reference rice genome (Nipponbare/japonica) was downloaded from the Phytozome database^{53,54}. The paired-end raw reads were first quality-evaluated using FastQC v0.11.9 (http://www.bioinformatics.babraham.ac.uk/projects/fastqc/)⁵⁵ and then mapped to the reference genome using BWA v0.7.15-r1140 with default parameters. Tandem Repeats Finder v4.09⁵⁶ was used with default parameters to scan the genome tandem repetition of DNA sequences.

To obtain high-confidence SNP markers that can differentiate the two parental alleles, we used the analysis strategy described below. First, for parental and F1 samples (with a mean depth of 14.3×), SNPs and SVs were predicted from whole-genome resequencing datasets using inGAP-family⁵⁷. Then, potential false-positive SNPs that come from sequencing errors, small indels, tandem repeats and SVs were identified and filtered by inGAP-family as described previously^{57,58}. Furthermore, those SNPs only present in one of the parents (each with two replicates, homozygous genotype), and with a heterozygous genotype in F1 plants (two replicates, reference allelic ratio larger than 0.3 and less than 0.7) and sequencing depths larger than three, were kept as high-confidence markers. In the end, we obtained a set of 267,753 high-confidence SNP markers for subsequent analysis.

For F2, TO, T1, and T2 samples (with a mean depth of 1.9×), the read count and genotype profile for SNPs were generated by inGAP-family, and then a sliding window-based method was used to construct the genome-wide genotype landscape across samples, with a window size of 200 kb and a sliding size of 100 kb.

Flow cytometry analysis: DNA content of DAPi-stained cell nuclei isolated from developing leaf blades and seed endosperms was determined by FACS using a PARTEC cell analyzer and Sysmex CyStain® UV Ploidy 05-5001 buffer (www.sysmex.de). The method for releasing nuclei into the buffer using manual chopping of leaf blade segments with a razor blade was that described in⁹. For endosperm nuclei, the pear-shaped developing seed was gently separated from the lemma and palea and allowed to release its milky endosperm into 0.5 ml of buffer solution using a pipette tip. Then, the turbid suspension containing nuclei was diluted in 3 ml of buffer and vortexed before FACS analysis.

Morphological trait phenotyping: Thirty-four to forty T2 plants derived from T1 plants (n=5) of each of four TO events (T314 15.1, 37.7 and T315 5.4 and 8.1) were grown in the containment greenhouse until maturity along with fifteen BRS-CIRAD 302 F1 control plants. All the plants flowered in a synchronous manner. The following traits were recorded at the time of harvesting: Master tiller height, tiller number, antepenultimate (n-1) leaf blade length and width, flag leaf blade length and width, master tiller panicle length, number of spikelets per panicle (average of three master tillers), filled grain frequency (%), and one-thousand grain weight.

Plant panicle fertility and pollen viability determinations: The panicle fertility of T1 and T2 plants as well as that of control BRS-CIRAD 302 plants grown along the transgenic plants, was determined by averaging the filled spikelet/total spikelet ratio of the three main tillers of each plant. The panicle fertilities of four T2 progeny plants of each of five T1 plants (i.e., 20 T2 plants) were analyzed per TO event. The panicle fertility of Kitaake T1 plants of two T314 and two T315 events grown alongside wild-type plants was estimated from the master tillers of five T1 plants. The pollen viability was determined by counting at least 1,500 Alexander's⁵⁹ solution-stained pollen grains released from the mature anthers of two flowers collected on four T2 plants in each of four selected TO events (T314 15.1 and 37.7, T315 5.4 and 8.1) and in control BRS-CIRAD 302. Viable pollen appear pink-colored whereas empty, unviable pollen grains appear green-colored. Alexander staining is known to overestimate pollen viability.

Microscopy analysis of meiotic products by clearing and callose detection by aniline blue staining: Ovaries and anthers collected at pre-anthesis stages (white or pale-yellow anthers) from independent T2 plants of apomictic events T314 15.1 and T314 37.7 (and of replicated samples collected on T3 plants of event T314 15.1) were fixed in Carnoy's or FAA fixatives and rinsed in 70% ethanol. Dissected ovaries were mounted in Hoyer's clearing medium and gently squashed by pressure on the coverslip to expose ovules. Callose detection using aniline blue staining was performed as described⁶⁰. Observation and imaging were done on a Zeiss Axioimager Z2 microscope equipped with DIC, CFP emission filter; 40× or 63× (NA1,4) oil immersion lenses, and a sCMOS camera (Hamamatsu ORCA Flash V2). Images were processed using ImageJ software.

Material for grain quality characterization: All oven-dried rice kernels were dehulled prior to the manual removal of embryos. Cargo rice seeds were then ground using a ball mill (Dangoumill 300, Prolabo, France). After determination of the moisture content of an aliquot of the fine-ground rice flours by thermogravimetry in a ventilated oven at 104 °C until constant weight was reached, samples were hermetically stored at ambient temperature until use.

Grain morphological feature: A flatbed scanner (EPSON Expression 10 000 XL) was used to acquire 800 ppi RGB images of about 50 kernels of each paddy and cargo rice seeds. The size and shape of kernels were obtained based on digital image processing to avoid the laborious measurement of individual grains with a calliper. The major axis (length) and minor axis (width) were determined from an ellipse that best fits the projected area of the binarized image of each kernel using the open source ImageJ program. For each sample, shape (length/width), thickness (E), and volume (V) were then estimated, assuming that the width was proportional to the thickness of the spheroid grain⁶¹.

Starch enzymatic determination: Without removal of negligible soluble oligosaccharides, a dried starchy sample aliquot (25 mg) was gelatinized at 90 °C for 1.5 h with NaOH (1 mL 0.02 N) prior to being hydrolyzed at 50 °C for 1.5 h with α-amyloglucosidase in a pH 4.5 citrate buffer. D-glucose was then indirectly evaluated via the production of gluconate through the reduction of nicotinamide adenine dinucleotide phosphate (NADPH) using hexokinase and glucose-6-phosphate-dehydrogenase enzymes by the UV method of spectrophotometry at 340 nm (R-Biopharm, 2021). The amount of starch was expressed in percent on a dry-weight basis (g starch per 100 g dried sample), using an estimated conversion factor of 0.9 between starch and D-glucose.

Amylose calorimetric determination: Amylose content was measured by differential scanning calorimetry with a DSC 8500 apparatus (Perkin Elmer, Norwalk, USA) using 9-10 mg db dried sample and 40 μL of 2% (W/v) L-α-lysophosphatidylcholine solution (Sigma Chemical Co., St Louis, USA) in a hermetically sealed micropan⁶². The energy of the amylose/lyso-phospholipid complex formation of the sample (J/g db) against a pure amylose standard (Avebe, Veendam, Netherlands) was estimated in duplicate during the cooling stage from 160 °C to 40 °C at a rate of 10 °C/min. The amount of amylose was expressed in percentage, on dry starch weight basis (g amylose per 100 g dried starch).

Statistical analyses: As most traits did not follow a normal distribution, the statistical significance of differences between transgenic lines and controls was determined using non-parametric Kruskal-Wallis tests and an α-risk of 0.05. Multiple pairwise comparison was performed according to Dunn's procedure with a Bonferroni-corrected significance level of the p-value. For panicle fertility, we used Duncan's test with a significance of 0.05%. The XLSTAT software (Addinsoft, Paris, France) was used for the statistical analyses.

Data availability: The whole-genome resequencing data of all individuals are available for download in the ArrayExpress database at EMBL-EBI under accession number E-MTAB-11931.

TABLE 1
Ploidy of T1 progeny plants of MiMe BRS-CIRAD 302 primary
transformants harboring the sgMiMe (T313), sgMiMe_pAtECS:
BBM1 (T314), and sgMiMe_pOsECS: BBM1 (T315) T-DNAs.

| | | TI | | | |
|---|---|---|---|---|---|
| | TO | plants | | | % |
| T-DNA construct | event | analyzed | 2n | 4n | diploids |
| 1313: | 4.3 | 27 | 0 | 27 | 0 |
| sgMiMe | 9.1 | 22 | 0 | 22 | 0 |
| | 12.1 | 20 | 0 | 20 | 0 |
| | 21 | 20 | 0 | 20 | 0 |
| T314: | 12.2 | 48 | 44 | 4 | 92 |
| sgMiMe_pAtECS: | 15.1 | 65 | 64 | 1 | 98 |
| BBM1 | 15.3 | 41 | 40 | 1 | 98 |
| | 16.1 | 37 | 30 | 7 | 81 |
| T1315: | 23.1 | 16 | 0 | 16 | 0 |
| sgMiMe_pOsECS: | 37.7 | 38 | 35 | 3* | 92 |
| BBM1 | 37.9 | 13 | 13 | 0 | 100 |
| | 44.1 | 12 | 11 | 1 | 92 |

The presence of the egg cell-specific promoter: BBM1 cassette was ascertained in all the T314 and T315 events. The number of plants analyzed varies according to T1 seed availability. Events selected for further analysis on the basis on both the score and confidence of diploid frequency appear in bold. * includes one 2n/4n chimeric plant; ** includes two 2n/4n chimeric plants

TABLE 2
Frequency of diploid plants in T2 and T3 progenies of selected
sgMiMe_pAtECS:BBM1 (T314) and sgMiMe_pOsECS:BBM1 (T315) events.

| | Generation | | | | | |
|---|---|---|---|---|---|---|
| | | | T2 | | T3 | |
| | | | Progenies of T1 plants | | Progenies of 3 T2 | |
| | T1 | | n > 40 T2 plants per | | plants n = 100 T3 plants | |
| | | n > 40 T1 | progeny | | per progeny | |
| T-DNA constructs | Event | Progenies | Average | Range (%) | Average | Range (%) |
| T314:sgMiMe_pAtECS:BBM1 | 151.1 | 98 | 96.6 | 95-100 | 98.7 | 98-99 |
| | 37.7 | 92 | 89.5 | 80-96 | 93.7 | 92-96 |
| T1315:sgMiMe_pOsECS:BBM1 | 3.2 | 96 | 93.2 | 86-100 | ND | ND |
| | 5.4 | 93 | 94.8 | 92-98 | 96.7 | 96-98 |
| | 8.1 | 95 | 93.8 | 83-11 | 92.7 | 92-94 |
| | 8.2 | 97 | 88.7 | 83-95 | ND | ND |

For T2 ploidy determination, at least 40 progeny plants from 5 individual T1 plants (i.e., at least 200 plants per event) were analyzed. For T3 ploidy determination, 100 plants from 3 individual T2 plants (i.e., 300 plants per event) were analyzed. Observations at the T1 generation are provided to facilitate interpretation of results.

TABLE 3
Phenotypes of T2 progenies of five T1 plants of sgMiMe_pAtECS:BBM1 (T314) and
sgMiMe_pOsECS:BBM1 (T315) events (n > 34 for each event) grown alongside
control BRS-CIRAD 302 F1 hybrid plants (n = 15) under controlled greenhouse conditions.

| | | Plant | | | | | |
|---|---|---|---|---|---|---|---|
| | | Traits | Number | n-1 | n-1 | Flag | Flag |
| Plant | | Plant | of | Leaf | Leaf | leaf | leaf |
| Materials | | Height | tillers | length | width | length | width |
| BRS-CIRAD | Mean | 84.8 a, b | 13.4 a | 501 a | 1.3 a | 3.19 c | 1.6 a, b, c |
| 302 | SD | 2.7 | 1.8 | 4.6 | 0.19 | 6.0 | 0.13 |
| T314 15.1 | Mean | 82.8 a | 14.4 a, b | 48.5 a | 1.4 a, b | 28.2 a, b, c | 1.6 a, b |
| | SD | 3.9 | 2.5 | 7.1 | 0.2 | 3.7 | 0.1 |
| T314 37.7 | Mean | 85.0 a, b | 15.2 | 46.0 a | 1.4 a, b | 26.9 a, b | 1.5 |
| | SD | 3.3 | 1.7 | 5.1 | 0.13 | 3.7 | .1 |
| T315 5.4 | Mean | 87.5 b | 14.2 a, b | 46.4 a | 1.6 c | 25.9 a | 1.6 b |
| | SD | 3.3 | 1.9 | 4.1 | 0.1 | 3.0 | 0.1 |
| T315 8.1 | Mean | 83.3 a | 13.8 a | 48.4 a | 1.5 b, c | 28.6 b, c | 1.6 b |
| | SD | 4.1 | 1.6 | 4.1 | 0.1 | 2.9 | 0.09 |

| | | Panicle | | | |
|---|---|---|---|---|---|
| | | traits | | | |
| | | Main | Spikelets | | 1000- |
| Plant | | Panicle | per | Penicle | grain |
| Materials | | length | panicle | fertility(%) | weight |
| BRS-CIRAD | Mean | 24.5 a, b, c | 176.4 a, b | 44.5 a | 21.1 a, b |
| 302 | SD | 1.4 | 7.9 | 3.7 | 0.2 |
| T314 15.1 | Mean | 24.3 a, b | 184 a, b | 33.8 a, b | 20.8 b |
| | SD | 1.5 | 11.1 | 2.0 | 0.3 |
| T314 37.7 | Mean | 23.6 a | 161.9 b | 27.0 c | 21.2 a |
| | SD | 1.0 | 8.2 | 1.4 | 0.4 |
| T315 5.4 | Mean | 24.7 b, c | 208.8 a | 35.5 b | 22.2 a |
| | SD | 0.9 | 12.6 | 2.4 | 0.3 |
| T315 8.1 | Mean | 25.4 c | 204.4 a | 33.1 b | 21.9 a, b |
| | SD | 1.3 | 8.6 | 2.2 | 0.2 |

Significant differences were tested using a non-parametric Kruskal-Wallis test with an α-risk of 0.05 followed by a pairwise comparison according to Dunn's test. For panicle fertility differences, Duncan's multiple range test was used. Significant differences with regards to the control at the risk of 0.05 appear in red.

### REFERENCES

1. Schnable, P. S. & Springer, N. M. Progress Toward Understanding Heterosis in Crop Plants. Annu. Rev. Plant Biol. 64, 71-88 (2013).
2. Huang, J.-Z., E, Z.-G., Zhang, H.-L. & Shu, Q.-Y. Workable male sterility systems for hybrid rice: Genetics, biochemistry, molecular biology, and utilization. Rice 7, 13 (2014).
3. Carman, J. G. Asynchronous expression of duplicate genes in angiosperms may cause apomixis, bispory, tetraspory, and polyembryony. Biological Journal of the Linnean Society 61, 51-94 (1997).
4. Ozias-Akins, P. & van Dijk, P. J. Mendelian genetics of apomixis in plants. Annu. Rev. Genet. 41, 509-537 (2007).
5. Ozias-Akins, P. & Conner, J. A. Clonal Reproduction through Seeds in Sight for Crops. Trends in Genetics 36, 215-226 (2020).
6. Underwood, C. J. & Mercier, R. Engineering Apomixis: Clonal Seeds Approaching the Fields. Annu. Rev. Plant Biol. 73, 201-225 (2022).

7 Hand, M. L. & Koltunow, A. M. G. The genetic control of apomixis: asexual seed formation. Genetics 197, 441-450 (2014).
8. d'Erfurth, I. et al. Turning meiosis into mitosis. PLOS Biol. 7, e1000124 (2009).
9. Mieulet, D. et al. Turning rice meiosis into mitosis. Cell Res. 26, 1242-1254 (2016).
10. Grelon, M., Vezon, D., Gendrot, G. & Pelletier, G. AtSPO11-1 is necessary for efficient meiotic recombination in plants. EMBO J. 20, 589-600 (2001).
11. Nonomura, K.-I. et al. The novel gene HOMOLOGOUS PAIR1NG ABERRATION IN RICE MEIOSIS1 of rice encodes a putative coiled-coil protein required for homologous chromosome pairing in meiosis. Plant Cell 16, 1008-1020 (2004).
12. Bai, X., Peirson, B. N., Dong, F., Xue, C. & Makaroff, C. A. Isolation and Characterization of SYN1, a RAD21-like Gene Essential for Meiosis in Arabidopsis. The Plant Cell 11, 417-430 (1999).
13. Cromer, L. et al. OSD1 promotes meiotic progression via APC/C inhibition and forms a regulatory network with TDM and CYCA1;2/TAM. PLOS Genet. 8, e1002865 (2012).
14. Marimuthu, M. P. A. et al. Synthetic clonal reproduction through seeds. Science 331, 876 (2011).
15. Ravi, M. & Chan, S. W. L. Haploid plants produced by centromere-mediated genome elimination. Nature 464, 615-618 (2010).
16. Yao, L. et al. OsMATL mutation induces haploid seed formation in indica rice. Nat Plants (2018) doi:10.1038/s41477-018-0193-y.
17. Wang, C. et al. Clonal seeds from hybrid rice by simultaneous genome engineering of meiosis and fertilization genes. Nature Biotechnology 1 (2019) doi: 10.1038/s41587-018-0003-0.
18. Li, X. et al. Single nucleus sequencing reveals spermatid chromosome fragmentation as a possible cause of maize haploid induction. Nat Commun 8, 991 (2017).
19. Khanday, I., Skinner, D., Yang, B., Mercier, R. & Sundaresan, V. A male-expressed rice embryogenic trigger redirected for asexual propagation through seeds. Nature 565, 91-95 (2019).
20. Khanday, I. & Sundaresan, V. Plant zygote development: recent insights and applications to clonal seeds. Current Opinion in Plant Biology 101993 (2021).
21. Steffen, J. G., Kang, I.-H., Macfarlane, J. & Drews, G. N. Identification of genes expressed in the Arabidopsis female gametophyte. The Plant Journal 51, 281-292 (2007).
22. Sprunck Stefanie et al. Egg Cell-Secreted EC1 Triggers Sperm Cell Activation During Double Fertilization. Science 338, 1093-1097 (2012).
23. Shao, T. et al. OsREC8 is essential for chromatid cohesion and metaphase I monopolar orientation in rice meiosis. Plant Physiol. 156, 1386-1396 (2011).
24. Bairu, M. W., Aremu, A. O. & Van Staden, J. Somaclonal variation in plants: causes and detection methods. Plant Growth Regulation 63, 147-173 (2011).
25. Luo, D. et al. A detrimental mitochondrial-nuclear interaction causes cytoplasmic male sterility in rice. Nature Genetics 45, 573-577 (2013).
26. Zhang, G., Lu, Y., Bharaj, T. S., Virmani, S. S. & Huang, N. Mapping of the Rf-3 nuclear fertility-restoring gene for WA cytoplasmic male sterility in rice using RAPD and RFLP markers. Theoretical and Applied Genetics 94, 27-33 (1997).
27. Zhang Q Y, Liu Y G, Zhang G Q, Mei M T. Molecular mapping of the fertility restorer gene Rf-4 for WA cytoplasmic male sterility in rice. Yi Chuan Xue Bao 29, 1001-1004 (2002).
28. Ramachandran, C. & Raghavan, V. Changes in Nuclear DNA Content of Endosperm Cells During Grain Development in Rice (Oryza sativa). Annals of Botany 64, 459-468 (1989).
29. Sabelli, P. A. Replicate and die for your own good: Endoreduplication and cell death in the cereal endosperm. Journal of Cereal Science 56, 9-20 (2012).
30. Sprunck Stefanie et al. Egg Cell-Secreted EC1 Triggers Sperm Cell Activation During Double Fertilization. Science 338, 1093-1097 (2012).
31. Anderson, S. N. et al. The Zygotic Transition Is Initiated in Unicellular Plant Zygotes with Asymmetric Activation of Parental Genomes. Developmental Cell 43, 349-358.e4 (2017).
32. Köhler, C., Dziasek, K. & Del Toro-De León, G. Postzygotic reproductive isolation established in the endosperm: mechanisms, drivers and relevance. Philosophical Transactions of the Royal Society B: Biological Sciences 376, 20200118 (2021).
33. Cheng, X. et al. The maternally expressed polycomb group gene OsEMF2a is essential for endosperm cellularization and imprinting in rice. Plant Communications 2, 100092 (2021).
34. Chen, Z. J. Genomic and epigenetic insights into the molecular bases of heterosis. Nature Reviews Genetics 14, 471-482 (2013).
35. Liu, J., Li, M., Zhang, Q., Wei, X. & Huang, X. Exploring the molecular basis of heterosis for plant breeding. Journal of Integrative Plant Biology 62, 287-298 (2020).
36. Sailer, C., Schmid, B. & Grossniklaus, U. Apomixis Allows the Transgenerational Fixation of Phenotypes in Hybrid Plants. Current Biology 26, 331-337 (2016).
37. Dapp, M. et al. Heterosis and inbreeding depression of epigenetic Arabidopsis hybrids. Nature Plants 1, 15092 (2015).
38. Lauss, K. et al. Parental DNA Methylation States Are Associated with Heterosis in Epigenetic Hybrids. Plant Physiology 176, 1627-1645 (2018).
39. d'Erfurth, I. et al. The cyclin-A CYCA1;2/TAM is required for the meiosis I to meiosis II transition and cooperates with OSD1 for the prophase to first meiotic division transition. PLOS Genet. 6, e1000989 (2010).
40. Cifuentes, M. et al. TDM1 Regulation Determines the Number of Meiotic Divisions. PLOS Genet. 12, e1005856 (2016).
41. Conner, J. A., Mookkan, M., Huo, H., Chae, K. & Ozias-Akins, P. A parthenogenesis gene of apomict origin elicits embryo formation from unfertilized eggs in a sexual plant. Proc Natl Acad Sci USA 112, 11205 (2015).
42. Conner, J. A., Podio, M. & Ozias-Akins, P. Haploid embryo production in rice and maize induced by PsASGR-BBML transgenes. Plant Reprod 30, 41-52 (2017).
43. Levings Charles S. The Texas Cytoplasm of Maize: Cytoplasmic Male Sterility and Disease Susceptibility. Science 250, 942-947 (1990).
44. Wing, R. A. Harvesting rice's dispensable genome. Genome Biology 16, 217 (2015).
45. Graham R. A Proposal for IRRI to Establish a Grain Quality and Nutrition Research Center. pp15 (2002).
46. Zhou, H., Liu, B., Weeks, D. P., Spalding, M. H. & Yang, B. Large chromosomal deletions and heritable small genetic changes induced by CRISPR/Cas9 in rice. Nucleic Acids Res 42, 10903-10914 (2014).
47. Miao, J. et al. Targeted mutagenesis in rice using CRISPR-Cas system. Cell Research 23, 1233-1236 (2013).
48. Christensen, A. H. & Quail, P. H. Ubiquitin promoter-based vectors for high-level expression of selectable and/or screenable marker genes in monocotyledonous plants. Transgenic Res. 5, 213-218 (1996).
49. Ouwerkerk, P. B., de Kam, R. J., Hoge, J. H. & Meijer, A. H. Glucocorticoid-inducible gene expression in rice. Planta 213, 370-378 (2001).
50. Ohnishi, T. et al. Distinct Gene Expression Profiles in Egg and Synergid Cells of Rice as Revealed by Cell Type-Specific Microarrays. Plant Physiology 155, 881-891 (2011).
51. Sallaud, C. et al. Highly efficient production and characterization of T-DNA plants for rice (Oryza sativa L.) functional genomics. Theor. Appl. Genet. 106, 1396-1408 (2003).
52. Yang, L. et al. Estimating the copy number of transgenes in transformed rice by real-time quantitative PCR. Plant Cell Rep 23, 759-763 (2005).
53. Goodstein, D. M. et al. Phytozome: a comparative platform for green plant genomics. Nucleic Acids Research 40, D1178-D1186 (2012).
54. Ouyang, S. et al. The TIGR Rice Genome Annotation Resource: improvements and new features. Nucleic Acids Research 35, D883-D887 (2007).
55. Li, H. & Durbin, R. Fast and accurate short read alignment with Burrows-Wheeler transform. Bioinformatics 25, 1754-1760 (2009).
56. Benson, G. Tandem repeats finder: a program to analyze DNA sequences. Nucleic Acids Research 27, 573-580 (1999).
57. Lian, Q., Chen, Y., Chang, F., Fu, Y. & Qi, J. inGAP-family: Accurate Detection of Meiotic Recombination Loci and Causal Mutations by Filtering Out Artificial Variants due to Genome Complexities. Genomics, Proteomics & Bioinformatics (2021) doi:10.1016/j.gpb.2019.11.014.
58. Qi, J., Chen, Y., Copenhaver, G. P. & Ma, H. Detection of genomic variations and DNA polymorphisms and impact on analysis of meiotic recombination and genetic mapping. Proc Natl Acad Sci USA 111, 10007 (2014).
59. Alexander, M. P. Differential Staining of Aborted and Nonaborted Pollen. null 44, 117-122 (1969).
60. Cao, L. et al. Arabidopsis ICK/KRP cyclin-dependent kinase inhibitors function to ensure the formation of one megaspore mother cell and one functional megaspore per ovule. PLOS Genetics 14, e1007230 (2018).
61. Cleva, M. S., Sampallo, G. M., Gonzalez Thomas, A. O., Acosta, C. A., Método para la determinación del volumen de una muestra de granos de arroz mediante el procesamiento digital de imágenes. Revista de Investigaciones Agropecuarias 39, 185-190 (2013).
62. Pérez, E. et al. Isolated starches from yams (Dioscorea sp) grown at the Venezuelan

```
SEQUENCES
SEQ ID NO: 1 OsBBM1 GenBank accession number: AAX95437.1
MASITNWLGFSSSSFSGAGADPVLPHPPLQEWGSAYEGGGTVAAAGGEETAAPKLEDELG
MQVQQETAAAAAGHGRGGSSSVVGLSMIKNWLRSQPPPAVVGGEDAMMALAVSTSASPPV
DATVPACISPDGMGSKAADGGGAAEAAAAAAAQRMKAAMDTFGQRTSIYRGVTKHRWTGR
YEAHLWDNSCRREGQTRKGRQVNAGGYDKEEKAARAYDLAALKYWGTTTTTNFPVSNYEK
ELDEMKHMNRQEFVASLRRKSSGFSRGASIYRGVTRHHQHGRWQARIGRVAGNKDLYLGT
FGTQEEAAEAYDIAAIKFRGLNAVTNFDMSRYDVKSIIESSNLPIGTGTTRRLKDSSDHT
DNVMDINVNTEPNNVVSSHFTNGVGNYGSQHYGYNGWSPISMQPIPSQYANGQPRAWLKQ
EQDSSVVTAAQNLHNLHHFSSLGYTHNFFQQSDVPDVTGFVDAPSRSSDSYSFRYNGTNG
FHGLPGGISYAMPVATAVDQGQGIHGYGEDGVAGIDTTHDLYGSRNVYYLSEGSLLADVE
KEGDYGQSVGGNSWVLPTP*
SEQ ID NO: 2
Arabidopsis thaliana BABY BOOM (AtBBM),
NCBI Protein Accession # NP_001332647.1; NP_197245
    1 MNSMNNWLGF SLSPHDQNHH RTDVDSSTTR TAVDVAGGYC FDLAAPSDES
  51 SAVQTSFLSP FGVTLEAFTR DNNSHSRDWD INGGACNNIN NNEQNGPKLE
101 NFLGRTTTIY NTNETVVDGN GDCGGGDGGG GGSLGLSMIK TWLSNHSVAN
151 ANHQDNGNGA RGLSLSMNSS TSDSNNYNNN DDVVQEKTIV DVVETTPKKT
201 IESFGQRTSI YRGVTRHRWT GRYEAHLWDN SCKREGQTRK GRQVYLGGYD
251 KEEKAARAYD LAALKYWGTT TTTNFPLSEY EKEVEEMKHM TRQEYVASLR
301 RKSSGFSRGA SIYRGVTRHH QHGRWQARIG RVAGNKDLYL GTFGTQEEAA
351 EAYDIAAIKF RGLSAVTNED MNRYNVKAIL ESPSLPIGSS AKRLKDVNNP
401 VPAMMISNNV SESANNVSGW QNTAFQHHQG MDLSLLQQQQ ERYVGYYNGG
451 NLSTESTRVC FKQEEEQQHE LRNSPSHMTN VDHHSSTSDD SVTVCGNVVS
501 YGGYQGFAIP VGTSVNYDPF TAAEIAYNAR NHYYYAQHQQ QQQIQQSPGG
551 DFPVAISNNH SSNMYFHGEG GGEGAPTESV WNDT
SEQ ID NO: 3
Brassica napus BABY BOOM1 (BnBBM1)
NCBI Protein Accession # NP 001302749; AAM33802
    1 mnnnwlgfsl spyeqnhhrk dvyssttttv vdvageycyd ptaasdessa iqtsfpspfg
  61 vvvdaftrdn nshsrdwdin gcacnnihnd eqdgpklenf lgrtttiynt nenvgdgsgs
121 gcygggdggg gslglsmikt wlrnqpvdnv dnqengnaak glslsmnsst scdnnndsnn
181 nvvaqgktid dsveatpkkt iesfgqrtsi yrgvtrhrwt gryeahlwdn sckregqtrk
241 grqvylggyd keekaarayd laalkywgtt tttnfpmsey ekeveemkhm trqeyvaslr
301 rkssgfsrga siyrgvtrhh qhgrwqarig rvagnkdlyl gtfgtqeeaa eaydiaaikf
361 rgltavtnfd mnrynvkail espslpigsa akrlkeanrp vpsmmmisnn vsesensasg
421 wqnaavqhhq gvdlsllhqh qeryngyyyn ggnlssesar acfkqeddgh hflsntqslm
481 tnidhqssvs ddsvtvcgnv vgyggyqgfa apvncdayaa sefdynarnh yyfaqqqqtq
541 qspggdfpaa mtnnvgsnmy yhgegggeva ptftvwndn
SEQ ID NO: 4
Brassica napus BABY BOOM2 (BnBBM2)
NCBI Protein Accession # AAM33801; NP_001303138
    1 mnnnwlgfsl spyeqnhhrk dvcsstttta vdvageycyd ptaasdessa iqtsfpspfg
  61 vvldaftrdn nshsrdwdin gsacnnihnd eqdgpklenf lgrtttiynt nenvgdidgs
121 gcygggdggg gslglsmikt wlrnqpvdnv dnqengngak glslsmnsst scdnnnyssn
181 nlvaqgktid dsveatpkkt iesfgqrtsi yrgvtrhrwt gryeahlwdn sckregqtrk
241 grqvylggyd keekaarayd laalkywgtt tttnfpmsey ekeieemkhm trqeyvaslr
301 rkssgfsrga siyrgvtrhh qhgrwqarig rvagnkdlyl gtfgtqeeaa eaydiaaikf
361 rgltavtnfd mnrynvkail espslpigsa akrlkeanrp vpsmmmisnn vsesennasg
421 wqnaavqhhq gvdlsllqqh qeryngyyyn ggnlssesar acfkqeddqh hflsntqslm
481 tnidhqssvs ddsvtvcgnv vgyggyqgfa apvncdayaa sefdynarnh yyfaqqqqtq
541 hspggdfpaa mtnnvgsnmy yhgegggeva ptftvwndn
SEQ ID NO: 5
Oryza sativa BABY BOOM1 (OsBBM1)
NCBI Protein Accession # XP_015616214
    1 masitnwlgf ssssfsgaga dpvlphpplq ewgsayeggg tvaaaggeet aapkledflg
  61 mqvqgetaaa aaghgrggss svvglsmikn wlrsqpppav vggedammal avstsasppv
121 datvpacisp dgmgskaadg ggaaeaaaaa aaqrmkaamd tfgqrtsiyr gvtkhrwtgr
181 yeahlwdnsc rregqtrkgr qvylggydke ekaaraydla alkywgtttt tnfpvsnyek
241 eldemkhmnr qefvaslrrk ssgfsrgasi yrgvtrhhqh grwqarigrv agnkdlylgt
301 fgtqeeaaea ydiaaikfrg lnavtnfdms rydvksiies snlpigtgtt rrlkdssdht
361 dnvmdinvnt epnnvvsshf tngvgnygsq hygyngwspi smqpipsqya ngqprawlkq
421 eqdssvvtaa qnlhnlhhfs slgythnffq qsdvpdvtgf vdapsrssds ysfryngtng
481 fhglpggisy ampvatavdq gqgihgyged gvagidtthd lygsrnvyyl segslladve
541 kegdygqsvg gnswvlptp
SEQ ID NO: 6
Oryza sativa BABY BOOM (OsBBM)
NCBI Protein Accession # XP_015634444
    1 matmnnwlaf slspqdqlpp sqtnstlisa aattttagds stgdvcfnip qdwsmrgsel
  61 salvaepkle dflggisfse qqhhhggkgg vipssaaacy assgssvgyl ypppsssslq
121 fadsvmvats spvvahdgvs gggmvsaaaa aaasgnggig lsmiknwlrs qpapqpaqal
181 slsmnmagtt taqgggamal lagagergrt tpaseslsts ahgattatma ggrkeineeg
241 sgsagavvav gsesggsgav veagaaaaaa rksvdtfgqr tsiyrgvtrh rwtgryeahl
301 wdnscrregq trkgrqvylg gydkeekaar aydlaalkyw gpttttnfpv nnyekeleem
361 khmtrqefva slrrkssgfs rgasiyrgvt rhhqhgrwqa rigrvagnkd lylgtfstqe
421 eaaeaydiaa ikfrglnavt nfdmsrydvk sildsaalpv gtaakrlkda eaaaaydvgr
481 iashlggdga yaahyghhhh saaaawptia fqaaaappph aaglyhpyaq plrgwckqeq
541 dhaviaaahs lqdlhhlnlg aaaaahdffs qamqqqhglg sidnaslehs tgsnsvvyng
601 dnggggggyi mapmsavsat atavasshdh ggdggkqvqm gydsylvgad ayggggagrm
661 pswamtpasa paatsssdmt gvchgaqlfs vwndt
SEQ ID NO: 7
Zea mays BABY BOOM1 (ZmBBM1) GRMZM2G366434
NCBI Protein Accession # NP_001147535
    1 masannwlgf slsgqdnpqp nadsspaagi disgasdfyg lptqqgsdgh 1gvpglrddh
  61 asygimeayn rvpqetqdwn mrgldynggg selsmlvgss gggggngkra vedsepkled
121 flggnsfvsd qdqsggylfs gvpiassans nsgsntmels miktwlrnnq vaqpqppaph
181 qpqpeemstd asgssfgcsd smgrnsmvaa ggssqslals mstgshlpmv vpsgaasgaa
241 sestssenkr asgamdspgs aveavprksi dtfgqrtsiy rgvtrhrwtg ryeahlwdns
301 crregqsrkg rqvylggydk edkaaraydl aalkywgttt ttnfpisnye keleemkhmt
361 rqeyiaylrr nssgfsrgas kyrgvtrhhq hgrwqarigr vagnkdlylg tfsteeeaae
421 aydiaaikfr glnavtnfdm srydvksile sstlpvggaa rrlkdavdhv eagatiwrad
481 mdgavisqla eagmggyasy ghhgwptiaf qqpsplsvhy pygqpsrgwc kpeqdaaaaa
541 ahslqdlqql hlgsaahnff qasssstvyn ggagasggyq glgggssflm psstvvaaad
601 qghsstanqg stcsygddhq egkligydaa mvataaggdp yaaarngyqf sqgsgstvsi
661 arangyannw sspfnngmg
SEQ ID NO: 8
Glycine max BABY BOOM1 (GmBBM1)
NCBI Protein Accession # XP 006586645.1; ADP37371
    1 mgsmnllgfs lspheehpss qdhsqttpsr fsfnpdgsis stdvaggcfd ltsdstphll
  61 nlpsygiyea fhrnnsintt qdwkenynsq nlllgtscnk qnmnqnqqqq pklenflggh
121 sfgeheqtyg gnsastdymf paqpvsaggg gsgggsnnnn nsnsiglsmi ktwlrnqppn
181 seninnnnes ggnirssvqq tlslsmstgs qsstslpllt asvdngesps dnkqpntsaa
241 ldstqtgaie taprksidtf gqrtsiyrgv trhrwtgrye ahlwdnscrr egqtrkgrqv
301 ylggydkeek aaraydlaal kywgtttttn fpishyekel eemkhmtrqe yvaslrrkss
361 gfsrgasiyr gvtrhhqhgr wqarigrvag nkdlylgtfs tqeeaaeayd vaaikfrgls
421 avtnfdmsry dvksilestt lpiggaakrl kdmeqvelsv dnghradqvd hsiimsshlt
481 qginnnyagg gtathhnwhn ahafhqpqpc ttmhypygqr inwckqeqqd nsdaphslsy
541 sdihqlqlgn ngthnffhtn sglhpmlsmd sasidnssss nsvvydgygg gggynvmpmg
601 tttavvasdg dqnprsnhgf gdneikalgy esvygsatds yhaharnlyy ltqqqsssvd
661 tvkasaydqg sacntwvpta ipthaprstt smalchgatt pfsllhe
SEQ ID NO: 9
Capsicum annuum BABY BOOM (CaBBM)
NCBI Protein Accession # XP_016568915
    1 mkmksmndds sssnnsnsnn nnhssaatns nnwlgfslsp hmkmevtnas etqqqqhphq
  61 qqfaqsfyls sspttmnvst asalcyennp fhsslsvmpl ksdgslcime alsrshadam
121 vqssspkled flggasqygs hereamalsl dslyyhqnde diqvhshhpy yspmhchgmy
181 qeslleetkp tqisncdaqm tgnemkswgh yaidqhindt csmvaaaaav aagggggtvg
241 cndlqslsls mnpgtqsscv tprqisptgl ecvaieskkr asakvaqkqp vhrksidtfg
301 qrtsqyrgvt rhrwtgryea hlwdnsckke gqtrkgrqvy lggydmedka araydqaalk
361 ywgpsthinf plenyqkele emknmtrqey vahlrrkssg fsrgasiyrg vtrhhqhgrw
421 qarigrvagn kdlylgtfst qeeaaeaydv aaikfrgvna vtnfdisryd vekimasnnl
481 pagelarrtk erepresiey nnisvhknee cvqnnnnngn itdwkmvlyq asnpsigsnn
541 yrnpsfsval qdligidsin nstshatild heqnkiganh fsnasslvts lgssreaspd
601 ktaaslvfak ptkfvvpttn vnacipsaql rpipvsmahl pvfaalnda
SEQ ID NO:10
Medicago truncatula BABY BOOM (MtBBM),
NCBI Protein Accession # XP_003624212
    1 masmnllgfs lspqeqhpst qdqtvasrfg fnpneisgsd vqgdhcydls shttphhsln
  61 lshpfsiyea fhtnnnihtt qdwkenynnq nlllgtscmn qnvnnnnqqa qpklenflgg
121 hsftdhqeyg gsnsysslhl pphqpeascg ggdgstsnnn siglsmiktw 1rnqppppen
181 nnnnnnesga rvqtlslsms tgsqssssvp llnanvmsge isssenkqpp ttavvldsnq
241 tsvvesavpr ksvdtfgqrt siyrgvtrhr wtgryeahlw dnscrregqt rkgrqvylgg
301 ydkeekaara ydlaalkywg tttttnfpis hyekeveemk hmtrqeyvas lrrkssgfsr
361 gasiyrgvtr hhqhgrwqar igrvagnkdl ylgtfstqee aaeaydvaai kfrglsavtn
421 fdmsrydvkt ilesstlpig gaakrlkdme qvelnhvnvd ishrteqdhs iinntshlte
481 qaiyaatnas nwhalsfqhq qphhhynann mqlqnypygt qtqklwckqe qdsddhstyt
541 tatdihqlql gnnnnnthnf fglqnimsmd sasmdnssgs nsvvygggdh ggyggnggym
601 ipmaiandgn qnprsnnnfg eseikgfgye nvfgtttdpy hagaarnlyy qpqqlsvdqg
661 snwvptaipt laprttnvsl cppftllhe
SEQ ID NO: 11
Cenchrus ciliaris CcASGR-BBM-like1
NCBI Protein Accession # ACD80125
    1 mgstnnwlrf vsfsggggak daaallplpp sprgdvdeag aepkledflg lqepsaaavg
  61 agrpfavggg assiglsmik nwlrsqpapa gpaagvdsmv laaaaastev agdgaeggga
121 vadavqqrka aavdtfgqrt siyrgvtkhr wtgryeahlw dnscrregqt rkgrqvylgg
181 ydkeekaara ydlaalkyrg tttttnfpms nyekeleemk hmsrqeyvas lrrkssgfsr
241 gasiyrgvtr hhqhgrwqar igsvagnkdl ylgtfstqee aaeaydiaai kfrglnavtn
301 fdmsrydvks iiessslpvq gapkrlkevp dqsdmginin gdsaghmtai nlltdgndsy
361 gaesygysgw cptamtpipf qfsighdhsr lwckpeqdna vvaalhnlhh lqhlpapvgt
421 hnffqpspvq dmtgvadass ppvesnsfly ngdvgyhgam ggsyampvat lvegnsagsg
481 ygveegtgse ifggrnlysl aqgssgantg kadayeswdp smlvisqksa nvtvchgapv
541 fsvwk
SEQ ID NO: 12
Pennisetum squamulatum PsASGR-BBM-like1
NCBI Protein Accession # ACD80127
    1 mgstnnwlrf asfsggggak daaallplpp sprgdvdeag aepkledflg lqepsaaavg
  61 agrpfavggg assiglsmir nwlrsqpapa gpaagvdsmv laaaaastev agdgaeggga
121 vadavqqrka aavdtfgqrt siyrgvtkhr wtgryeahlw dnscrregqt rkgroggydk
181 eekaaraydl aalkyrgttt ttnfpmsnye keleemkhms rqeyvaslrr kssgfsrgas
241 iyrgvtrhhq hgrwqarigs vagnkdlylg tistqeeaae aydiaaikfr glnavtnfdm
301 srydvksiie ssslpvggtp krlkevpdqs dmginingds aghmtainll tdgndsygae
361 sygysgwcpt amtpipfqfs nghdhsrlwc kpeqdnavva alhnlhhlqh lpapvgthnf
421 fqpspvqdmt gvadassppv esnsflyngd vgyhgamggs yampvatlve gnsagsgygv
481 eegtgseifg grnlyslsqg ssgantgkad ayeswdpsml visqksanvt vchgapvfsv
541 wk
SEQ ID NO: 13
Pennisetum squamulatum PsASGR-BBM-like2
NCBI Protein Accession # ACD80124.2
    1 mgstnnwlrf asfsggggak daaallplpp sprgdvdeag aepkledflg lqepsaaavg
  61 agrpfavggg assiglsmir nwlrsqpapa gpaagvdsmv laaaaastev agdgaeggga
121 vadavqqrka aavdtfgqrt siyrgvtkhr wtgryeahlw dnscrregqt rkgrqvylgg
181 ydkeekaara ydlaalkyrg tttttnfpms nyekeleemk hmsrqeyvas lrrkssgfsr
241 gasiyrgvtr hhqhgrwqar igsvagnkdl ylgtfstqee aaeaydiaai kfrglnavtn
301 fdmsrydvks iiessslpvg gtpkrlkevp dqsdmginin gdsaghmtai nlltdgndsy
361 gaesygysgw cptamtpipf qfsnghdhsr lwckpeqdna vvaalhnlhh lqhlpapvgt
421 hnffqpspvq dmtgvadass ppvesnsfly ngdvgyhgam ggsyampvat lvegnsagsg
481 ygveegtgse ifggrnlysl sqgssgantg kadayeswdp smlvisqksa nvtvchgapv
541 fsvwk
SEQ ID NO: 14
Rosa canina BABY BOOM1 (RcBBM1)
NCBI Protein Accession # AGZ02154.1
    1 mnmnwlgfsl spqeddhapi shqladgetl asrlgfnsne iqgagggtdv sgggssecfd
  61 vtnsdstasv nhhltpsifg iheaaafnrn ndhihsqdwn mkgagmnssd snnykrtsss
121 dlstmlmgsn tsttstsysi isqqanlenh hqqlpklenf lgrhsfadhd sssaghdymf
181 dmnngpgpvs snvmntktns nniglsmikt wlrnqpsqpr dnhhqleges knskesrnqp
241 qsslslsmgt gslqtvttat aggaatgett sssdkntkqs pvvtattttg tdaqtqstgg
301 aieavprkai dtfgqrtsiy rgvtrhrwtg ryeahlwdns crregqtrkg rqvylggydk
361 edkaaraydl aalkywgttt ttnfpissye keidemkpmt rqeyvaslrr kssgfsrgas
421 iyrgvtrhhq hgrwqarigr vagnkdlylg tfstqeeaae aydiaaikfr glnavtnfdm
481 srydvksile ssalpittga takrlkdvqq qqppppadhh hqimlssvld hhgqvirsss
541 stehdimsnv ysaygsygaq qgcswptlaf ngaqaqaaaa phqapfaagi ngmqlhyspy
601 gygygnahaq rvwckqeqdt nsnqersfhh qdddhlrqql qlggthnffh dhdqqqqqqq
661 qqtsglmglm dssaasmehs sgsnsviysg gdhhgnnngy gssttgtggg yimpmvmstv
721 vanddqnqad gnnnningfg dgddqeanik aqqlgydhhp qnmflgssst idpayqhhas
781 nrnlyyhlpv qddqhesssv avatsstton mnwvptavpt lahptftvwn dt
SEQ ID NO: 15
Rosa canina BABY BOOM2 (RcBBM2)
NCBI Protein Accession # AGZ02155.1
    1 mnmnwlgfsl spqeddhapi shqladqetl asrlgfnsne ihgagggtdv sgggssecfd
  61 ltnsdstasv nhhltpsifg iheaaafnrn ndhihsqdwn mkgagmnssd snnykrtsss
121 dlstmlmgsn tttstscsii sqqanlenhh qqlpklenfl grhsfadhdr ssaghdymfd
181 mnngpgpvss nvmntktnsn niglsmiktw lrnqpsqprd nhhqlegesk nskesrnqpq
241 sslslsmgtg slqtvttata ggaataatge ttcssdkntk qspvvtattt tgtdaqtqst
301 ggaieavprk aidtfgqrts iyrgvtrhrw tgryeahlwd nscrregqtr kgrqvylggy
361 dkedkaaray dlaalkywgt ttttnfpiss yekeidemkp mtrqeyvasl rrkssgfsrg
421 asiyrgvtrh hqhgrwqari grvagnkdly lgtfstqeea aeaydiaaik frglnavtnf
481 dmsrydvksi lessalpitt gatakrlkev qqqqppppad hhhqimlssv ldhhgqiirs
541 ssstehdims nvysaygsyg aqqgcswptl afnqaqaqaa aaphqapfaa gingmqlhys
601 pygygygnah aqrvwckqeq dtnsnqessf hhqdddhlrq qlqlggthnf fhdhdqqqts
661 glmglmdssa asmehssgsn sviysggdhh gnnngygsst tgtgggyimp mvmstvvand
721 dqnqadgnnn ningfedgdd qeanikaqql gydhhhqnmf lgssstidpa yqhhasnrnl
781 yyhlpvqddq hesssvavat ssttonmnwv ptavptlahp tftvwndt
SEQ ID NO: 16
Ricinus communis AIL6
NCBI Protein Accession # XP 015583464
    1 mapattnwls fslspmemlr sstesqfisy egsstatpsp hyfidnfyan gwgnpkeaqg
  61 attmaaetsi ltsfidpeth hqqvpkledf lgdsssivry sdnsqtdtqd sslthiydqg
121 saayfseqqd lkaiagfqaf stnsgsevdd sasiarthlg gefmghsids sgndqlggfs
181 nctaannals lavnnnnnnn gnqsatnskt iapviesdcp kkiadtfgqr tsiyrgvtrh
241 rwtgryeahl wdnscrregq arkgrqgalf flfspsssyh lslfvacffn yssvkilgiy
301 axsaiphghv lyffqvtnyt keldemkyvs kqefiaslrr kssgfsrgas iyrgvtrhhq
361 qgrwqarigr vagnkdlylg tfateeeaae aydiaaikfr gmnavtnfem srydveaimk
421 salpiggaak rlklsleseq kpnlnheqqp qgsssnsssn nisfasmppv taipcgipfe
481 nttaqlyhhh hhhhhhqhhn lfhhlqttnn nlggttdiss gsttssmatt msmlpqtaef
541 flwphhqsy
SEQ ID NO: 17
Elaeis guineensis EgAP2-1
NCBI Protein Accession # AAV98627; NP_001290493
    1 mdmdtshswl afslsyhqpy llealssapp hggggmtaee rggsaevaam avvgpkledf
  61 lggcgepmgr yaggetgdag giydselkhi aagylqglpa teqqdsemak vaapaesrka
121 vetfgqrtsi yrgvtrhrwt gryeahlwdn scrregqsrk grqvylggyd keekaarayd
181 laalkywgpt tttnfpisny ekeleemknm trqefvaslr rkssgfsrga siyrgvtrhh
241 qhgrwqarig rvagnkdlyl gtfstqeeaa eaydiaaikf rglnavtnfd isrydvksia
301 nsnlpiggmt grpskatess pssssdamtv eakqlldgrd psaslgfaal pikhdqdfws
361 lfalqqqqqq qqqqsnqasg fglfssgvtm dfstasngvi sqgcggslvw nggvvgqqqe
421 qsqnnscssi pyatpiafgg nyegssyvgs wvtpppsyyh epakpnvavf qtpifgme
SEQ ID NO: 18
Populus trichocarpa BABY BOOM1 (PtBBM1)
NCBI Protein Accession # XP_002316179
    1 masmnnwlgf slshqelpss qsdhhqdhsq ntdsrlgfhs deisgtnvsg ecfdltsdst
  61 apslnlpatf gileafrnnq pqdwnmkslg mnpdtnykta sglpifmgts cnsqtidqnq
121 epklenflgg hsfgnhehkl ngcntmydtt gdyvfqncs1 qlpseatsne rtsnngggdn
181 knssiglsmi ktwlrnqpap tqqdtnnknn ggaqslslsm stgsqsaasa lpllavnggv
241 nntggdqsss dnnkqqkstt psldsqtgai esvprksidt fgqrtsiyrg vtrhrwtgry
301 eahlwdnscr regqtrkgrq vylggydkee kaaraydlaa lkywgttttt nfpitnyeke
361 ieemkhmtrq eyvaslrrks sgfsrgasiy rgvtrhhqhg rwqarigrva gnkdlylgtf
421 stqeeaaeay diaaikfrgl navtnfdmsr ydvnsiless tlpiggaakr lkeaehaeia
481 mdiaqrtddh dnmgsqltdg issygavqhg wptvafqqaq pfsmhypygq rlwckqeqds
541 dnrsfqelhq lqlgnthnff qpsvlhnlvs mdsssmehss gsnsvvyssg vndgtstgtn
601 ggyqgigygs sagyavpmat visnndnnhn qgngygdgdq vkalgyenmf spsdpyharn
661 lhylsqqpsa ggikasaydq gsacynwvpt avptiaaars nnmavchgaq pftvwndgt
SEQ ID NO: 19
Populus trichocarpa BABY BOOM2 (PtBBM2)
NCBI Protein Accession # XP_002311259
    1 mastnnwlgf slspqelpss qsdhhdhpqn tdsrlrfhsd eisgtdvsge sfdltsdsta
  61 pslnlpasfg ileafrnnqs qdwnnmkrsg inedtsyntt sdvpifmgss cnsqnidqnq
121 epklenflgg hsfgnhehkl nvcstmygst ghymfhncsl qlpsedasne rtssnggadt
181 sinnnntnss iglsmiktwl knqpaptqqd tnnksnggaq slslsmstgs qsgsdlplla
241 vngggnrtrg eqsssdnnkq qkttpsldsq tgaievvprk sidtfgqrts iyrgvtrhrw
301 tgryeahlwd nscrregqtr kgroggydke dkaaraydla alkywgtttt tnfpmsnyek
361 eieemkhmtr qehvaslrrk ssgfsrgasi yrgvtrhhqh grwqarigrv agnkdlylgt
421 fstqeeaaea ydiaaikfrg Inavtnfdmn rydvnsimes stlpiggaak rlkeaehaei
481 ttrvqrtddh dstssqltdg isnygtaahh gwptiafqqa qaftmhypyg qrlwckqeqd
541 sdnhsfqelh qlqlgntqnf lqpsvlhnvm smesssmehs sgsdsvmyss gghdgtgtgt
601 ngsyqgigyg sntgyaipma tviandvntq dqgngygdge vkalgyenmf sssdpyharn
661 lyylsqqssa gvikasaydq gstcnnwlpt avptiaarsn nmavchgapt ftvwnest
SEQ ID NO: 20
Larix gmelinii var. olgensis x Larix kaempferi BABY BOOM (BBM); KJ004517
NCBI Protein Accession #AHH34920.1
    1 mgstsnwlaf slsphltvdm pdstqprsts aasnhsrhhn dfsngtvhdc yelhptdtmq
  61 mplrpdgslc ilealdrtqn nqdwqlksle npgsmdlesd vqsqqmmkse lsilaggsse
121 qmsasigrhk nvdqegpkle dflggaslrg hyndartdsi ygnddafdek mmapglrdvv
181 pnclngfdvt dtelssgskk tdqnqdstrn insignslvq dsydqnsndq ymfqdcslql
241 ppnsgannmi glsmiktwlr sqpcpenkmn aatnsstpts akdqslgnlt niqslslsms
301 pgsqssspla lpvqyqntna dspsseskkr slekqslvsv eatprksidt fgqrtsiyrg
361 vtrhrwtgry eahlwdnscr regqtrkgrq vylggydkee kaaraydlaa lkywgptttt
421 nfptgnyeke leemkhmtrq eyvaslrrks sgfsrgasiy rgvtrhhqhg rwqarigrva
481 gnkdlylgtf ssqeeaaeay diaaikfrgl navtnfdmtr ydvnsiless tlpiggaaak
541 rikdaepsdp svdgrrtdde isstissqia dtltsygnaa ypnghagwpi iafqqqtnph
601 apafysqqra aagwckgehn niqnhdlqlh fqsstqnflq psmmtsnant vlhnlmnles
661 saqldgtntn snsglfsnis gnlagnslqm anspipsgit vcdsartpfs tendgsstkn
721 ssyndnmlsn sdpfarglyy lsqhspsvvk anyenaaynn wmtpavqtla prpnltvcha
781 piftvwndt
SEQ ID NO: 21: Arabidopsis DD45 promoter
CCTCTTTGTCACCGTCACTCTTCTCCTCGTTCTCAACGTCTCCAGCAGAGCACTCCCGCCCGTGGCGGATTCCAC
CAACATAGCGGCTAGACTAACCGGAGGAGGACTGATGCAGTGTTGGGATGCACTCTACGAGCTGAAGTCATGTAC
TAATGAGATCGTTCTCTTCTTTCTCAACGGTGAGACCAAACTCGGCTACGGTTGCTGCAACGCCGTTGATGTCAT
TACCACTGATTGTTGGCCGGCGATGCTTACTTCTCTTGGCTTTACACTGGAGGAAACCAATGTCCTCCGTGGTTT
CTGTCAATCTCCGAACTCCGGCGGTTCTTCTCCAGCTCTTTCCCCTGTCAAACTTTGATAAATGTTCCTCGCTGA
CGTAAGAAGACATTAGTAATGGTTATAATATATAGCTTTCTATGAATGTATGGTGAGAAAATGTCTGTTCACTGA
TTTTGAGTTTG
GAATAAAAGCATTTGCGTTTGGTTTATCATTGCGTTTATACAAGGACAGAGATCCACTGAGCTGGAATAGCTTAA
AACCATTATCAGAACAAAATAAACCATTTTTTGTTAAGAATCAGAGCATAGTAAACAACAGAAACAACCTAAGAG
AGGTAACTTGTCCAAGAAGATAGCTAATTATATCTATTTTATAAAAGTTATCATAGTTTGTAAGTCACAAAAGAT
GCAAATAACAGAGAAACTAGGAGACTTGAGAATATACATTCTTGTATATTTGTATTCGAGATTGTGAAAATTTGA
CCATAAGTTTAAATTCTTAAAAAGATATATCTGATCTAGATGATGGTTATAGACTGTAATTTTACCACATGTTTA
ATGATGGATAGTGACACACATGACACATCGACAACACTATAGCATCTTATTTAGATTACAACATGAAATTTTTCT
GTAATACATGTCTTTGTACATAATTTAAAAGTAATTCCTAAGAAATATATTTATACAAGGAGTTTAAAGAAAACA
TAGCATAAAGTTCAATGAGTAGTAAAAACCATATACAGTATATAGCATAAAGTTCAATGAGTTTATTACAAAAGC
ATTGGTTCACTTTCTGTAACACGACGTTAAACCTTCGTCTCCAATAGGAGCGCTACTGATTCAACATGCCAATAT
ATACTAAATACGTTTCTACAGTCAAATGCTTTAACGTTTCATGATTAAGTGACTATTTACCGTCAATCCTTTCCC
ATTCCTCCCACTAATCCAACTTTTTAATTACTCTTAAATCACCACTAAGCTTCGAATCCATCCAAAACCACAATA
TAAAAACAGAACTCTCGTAACTCAATCATCGCAAAACAAAACAAAACAAAACAAAAACCCCAAAAAGAAAGAATA
SEQ ID NO: 22: Rice egg cell-specific promoter sequence from LOC_Os03g18530 OsECAI
gene
ATGGAATGATGGATGAATGTTCACGTTCTTGAGTTCCTAAATGGTACTAATTTTGCAAAAACTTTCTATATGTGT
TTTTTGTTAAGAATGTTGTTTTAAACCCATCTTTTCACTTTATAATATTTAATTAAATCGTTCGTACCCTCGAAT
AGTTATTGCAAATTATACTTAACTATTCAGTCATTCAGCACAAAAGAACAGGGCCATGAAATTGTAATACTAGTA
CATTTCTGTTCTTTTCTTTTCTTTTTGAGGTTGTCTGAAACACCTGTATCTTAAACTATCGCAGACTAGCCAATG
AGTCGTACTCACCTGAAACTGAAACCAAGTGATTAACCAAGCTGGTTCGACAGTAATTCCATCCATAATGCAGCT
CCGGAGCCCTTCATATCCTGCATGTTACTCAAACAACATCCCCACCTCCTCATTTCCTCTCCCCTATTGCATTGC
ATAATTGCAGAAGATTAAGCCGCTAATGCATAATTACACATTATTTGTGTCCACTAATTTTCCCTTTCCCACACG
CTACGAAACTCAAAAGCCGGCCTCCTCGCCTCCTTCCCTGAACGTTACTAATCGCGTCATGTATAAATACAGAGC
TTGCCCACGCACCGGCACATTGCATCGCACTACGCACATCTACACGATACCCAAGCAGCAAAGCTAGAAAGAAAA
ACC
SEQ ID NO: 23. Arabidopsis EGG CELL 1.1 (EC1.1) promoter sequences from AT1G76750
gene
GTTGCCTTATGATTTCTTCGGTTTCAAGATGATCAAATAGTTATAGATTTCATGCTCACACATGCTCATTAGATG
TGTACATACTTTACTTACCCAAATCTATTTTCTCGCAAAGATTTTGATGGTAAAGCTGATTTGGTTCTATTGAAC
TAAATCAAACGAGTTTCAGACTGAGTGATTCTAATCCGGCCCATTAGCCCCTAAACAGACCCACTAATTACGCAG
CTTTTAATAGAGTAATTACACCTAGTTTACCCACTAAACCACTAAGCACTAATTATCTCACAATCTAATGAGCTT
CCCTCGTAATTACTTGGGCTTTCACTCTACCATTTATTTGTAACAGTCAAGTCTCTACTGTCTCTATATAAACTC
TCTAAAGTTAACACACAATTCTCATCACAAACAAATCAACCAAAGCAACTTCTACTCTTTCTTCTTTCGACCTTA
TCAATCTGTTGAGAA
SEQ ID NO: 24
Rice OSD1; Locus# LOC_Os02g37850 CDS Sequence
Os02g37850.2
atgcctgaagtgagaaattccggcggtagggcggcgctcgccgacccctcgggtggtggg
ttctttatcaggaggacgacgtcgccgccgggagccgtggcggtcaagccgctggctcgg
cgggccctgccgccgacgagcaacaaggagaacgtgccgccgtcctgggctgtgaccgtg
agggctacacccaagaggaggagccccctgcccgagtggtacccgaggagcccactccgc
gacatcacgtcagtcgtcaaggcagttgagaggaaaagtcgcctcggaaatgctgcggtt
cggcagcagatccagttgagtgaagattcttcacgatctgtggatccagcaactccagta
caaaaagaagaaggtgtccctcaaagcacaccaacaccaccaactcaaaaggccctggat
gctgctgccccttgtcctggctcaacccaagctgttgcaagcacatcaacagcttacttg
gccgagggcaagccgaaggcatcatcttcttctccatctgactgctcctttcagacacca
tccagaccaaatgatccagctcttgctgatctcatggagaaggaactgtccagctccata
gagcagatagagaagatggtaaggaagaacctcaagagagctccgaaggctgctcagcct
tccaaggtgaccatccagaagcgcaccctgttgtccatgagatga
SEQ ID NO: 25
Rice OSD1; Locus# LOC_Os02g37850 Protein Sequence
MPEVRNSGGRAALADPSGGGFFIRRTTSPPGAVAVKPLARRALPPTSNKENVPPSWAVTV
RATPKRRSPLPEWYPRSPLRDITSVVKAVERKSRLGNAAVRQQIQLSEDSSRSVDPATPV
QKEEGVPQSTPTPPTQKALDAAAPCPGSTQAVASTSTAYLAEGKPKASSSSPSDCSFQTP
SRPNDPALADLMEKELSSSIEQIEKMVRKNLKRAPKAAQPSKVTIQKRTLLSMR*
SEQ ID NO: 26
Arabidopsis OSD1; Locus#AT3G57860 CDS Sequence
AT3G57860.1
    1 ATGCCAGAAG CAAGAGATCG AACCGAGAGG CCTGTGGATT ACTCGACTAT
  51 ATTTGCCAAC CGACGGAGAC ATGGIATITT ACTTGACGAG CCAGATTCAC
101 GGCTTAGTTT GATTGAATCT CCGGIGAATC CAGATATTGG GTCTATTGGT
151 GGAACGGGCG GGCTIGTGAG AGGCAATITC ACTACATGGA GGCCTGGTAA
201 TGGCAGAGGT GGTCACACTC CATTTAGATT GCCACAGGGA AGAGAGAATA
251 TGCCCATAGT GACCGCTAGG CGTGGAAGAG GTGGTGGITT GTTGCCTTCT
301 TGGTATCCAA GAACACCICT ACGCGACATA ACTCATATTG TGCGGGCTAT
351 TGAGAGAAGA AGAGGAGCTG GGACTGGAGG AGACGATGGC CGAGTIATTG
401 AGATCCCAAC TCATCGACAA GITGGTGITC TTGAATCTCC AGIACCACTG
451 TCAGGAGAAC ACAAATGCTC GATGGTCACT CCTGGACCAT CTGTGGGATT
501 CAAGCGTAGT TGCCCACCAT CAACTGCTAA AGTTCAAAAG ATGTTACTTG
551 ACATCACTAA AGAGATAGQT GAGGAAGAAG CTGGCTTCAT CACACCCGAG
601 AAGAAGCTAC TCAATTCTAT TGACAAAGTT GAGAAAATTG TGATGGCGGA
651 GATCCAGAAG TIGAAGAGCA CICCICAAGC TAAAAGGGAA GAGCGGGAGA
701 AGAGGGTGCG GACTITAATG ACTATGCGAT GA
SEQ ID NO: 27
Arabidopsis OSD1; Locus#AT3G57860 Protein Sequence

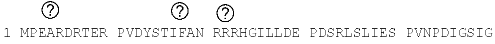

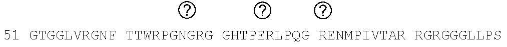

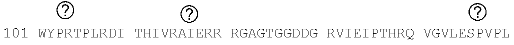

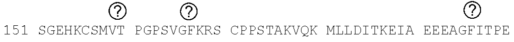

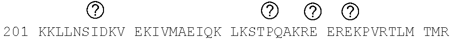





SEQ ID NO: 28
Rice PAIR1, Locus# LOC_Os03g01590 CDS Sequence
>LOC_Os03g01590.1
ATGAAGCTTAAGATGAACAAGGCCTGCGACATCGCCTCCATCTCCGTCCTCCCTCCCCGG
AGGACCGGAGGGAGCAGCGGCGCGTCGGCTTCCGGTTCCGTGGCGGTGGCGGTGGCGTCT
CAGCCGCGGTCGCAGCCGCTCTCGCAGTCGCAGCAGTCCTTCTCGCAGGGCGCCTCCGCC
TCGCTCTTGCACTCGCAGTCGCAGTTCTCGCAGGTCTCCCTCGACGACAACCTCCTCACC
CTCCTCCCTTCCCCCACCCGCGATCAGAGATTTGGCTTGCATGATGACTCATCCAAGAGG
ATGTCCTCTTTACCAGCCAGTTCAGCTTCTTGCGCGCGAGAAGAGTCTCAGCTGCAACTG
GCAAAATTACCAAGCAACCCAGTGCACCGCTGGAACCCCTCCATTGCAGATACTAGATCA
GGTCAGGTTACTAATGAGGATGTTGAGCGCAAATTTCAGCATCTGGCAAGCTCAGTACAT
AAGATGGGGATGGTGGTAGACTCAGTCCAAAGTGACGTAATGCAGTTAAACAGAGCCATG
AAGGAGGCATCATTAGATTCTGGTAGCATACGGCAAAAGATTGCTGTCCTTGAAAGCTCA
CTTCAGCAAATTCTTAAGGGACAAGACGATCTCAAAGCACTCTTTGGAAGCAGCACAAAA
CACAATCCTGATCAGACAAGTGTTCTGAATTCTCTAGGCAGCAAATTGAATGAGATATCC
TCGACCCTTGCAACCTTGCAGACACAAATGCAAGCAAGACAACTGCAGGGTGATCAGACA
ACTGTTCTGAATTCTAATGCCAGCAAATCGAATGAGATATCCTCGACTCTTGCAACCCTG
CAGACACAAATGCAAGCAGATATAAGACAACTGCGGTGTGACGTCTTCAGAGTTTTTACA
AAAGAGATGGAGGGGGTTGTTAGAGCTATCAGGTCTGTCAATAGTAGGCCTGCTGCAATG
CAAATGATGGCAGACCAGAGTTACCAAGTACCAGTTTCAAATGGATGGACCCAGATTAAC
CAGACACCAGTAGCAGCTGGAAGGTCTCCAATGAACCGAGCACCAGTAGCAGCTGGAAGG
TCCCGGATGAACCAATTACCTGAAACAAAAGTGCTTTCTGCACATTTGGTTTATCCTGCA
AAGGTGACAGATCTGAAGCCAAAGGTGGAGCAGGGAAAGGTAAAAGCAGCTCCACAAAAG
CCGTTTGCTTCGAGCTACTACAGGGTGGCACCTAAACAGGAAGAGGTAGCGATTAGAAAG
GTCAATATACAAGTGCCAGCAAAGAAGGCACCAGTCAGCATAATCATCGAGTCGGATGAT
GACAGTGAAGGACGTGCGTCCTGCGTGATTTTGAAGACAGAAACAGGTAGCAAGGAGTGG
AAAGTGACAAAGCAAGGCACCGAAGAGGGCCTGGAGATCCTGCGGAGGGCGAGGAAGAGG
AGGAGGAGAGAGATGCAGTCCATCGTGCTCGCATCCTAG
SEQ ID NO: 29
Rice PAIRI, Locus# LOC_Os03g01590 Protein Sequence
MKLKMNKACDIASISVLPPRRTGGSSGASASGSVAVAVASQPRSQPLSQSQQSFSQGASA
SLLHSQSQFSQVSLDDNLLTLLPSPTRDQRFGLHDDSSKRMSSLPASSASCAREESQLQL
AKLPSNPVHRWNPSIADTRSGQVTNEDVERKFQHLASSVHKMGMVVDSVQSDVMQLNRAM
KEASLDSGSIRQKIAVLESSLQQILKGQDDLKALFGSSTKHNPDQTSVLNSLGSKLNEIS
STLATLQTQMQARQLQGDQTTVLNSNASKSNEISSTLATLQTQMQADIRQLRCDVERVET
KEMEGVVRAIRSVNSRPAAMQMMADQSYQVPVSNGWTQINQTPVAAGRSPMNRAPVAAGR
SRMNQLPETKVLSAHLVYPAKVTDLKPKVEQGKVKAAPQKPFASSYYRVAPKQEEVAIRK
VNIQVPAKKAPVSIIIESDDDSEGRASCVILKTETGSKEWKVTKQGTEEGLEILRRARKR
RRREMQSIVLAS*
SEQ ID NO: 30
Arabidopsis SPO11-2; Locus# AT1G63990 CDS Sequence
>AT1G63990.1
      1 ATGGAGGAAA GITCAGGACT ATCATCGATG AAGTICTICT CCGATCAACA
    51 CCTCTCTTAC GCTGACATTC TTCTTCQTCA CGAAGCTCGT GQTAGAATCG
  101 AAGTTTCIGT TCTCAATCTC CTCCGAATTT TGAACTCCCC AGATCCAGCT
  151 ATCTCCGATC TCTCTCTGAT CAATAGGAAG AGAAGCAATA GTTGTATAAA
  201 CAAAGGGATA CICACAGATG TTICATATAT ATICCTCTCT ACTTCGTTTA
  251 CTAAGAGITC ATTGACGAAT GCTAAAACAG CTAAAGCTIT TGTTCGTGTG
  301 TGGAAAGITA TGGAAATATG CTTTCAGATT CTGCTICAAG AGAAACGAGT
  351 CACACAAAGA GAGCICTTCT ATAAGTTGCT TTGTGATTCA CQTGATTACT
  401 TITCATCICA GATTGAAGTT AACAGAAGTG TCCAAGATGT GGTAGCACIT
  451 CIACGTIGTA GIAGATACAG TCITGGTATT ATGGCITCAA GCAGAGGCCT
  501 TGTTGQTGGA AGGTTATITC TACAAGAACC AGGTAAGGAA GQTGTAGAIT
  551 GITCGGCCTG TGGTICTICA GGITTIGCTA TAACTGGAGA CTTGAATTIG
  601 CTAGACAATA CCATCATGAG AACTGATGCT CGITATATCA TTATTGTGGA
  651 AAAGCATGCG ATCTITCATC GGCTCGTGGA AGATCGTGTG TTCAATCACA
  701 TICCITGCGT GITCATCACA GCGAAAGGGT ATCCGGATAT TGCCACAAGG
  751 TITTICCICC ACCGGATGAG CACAACTTTT CCIGAICTGQ CAATTCTTGT
  801 TCTAGTIGAT TGGAATCCAG CIGGGTTAGC TATACTATGC ACCTTCAAGT
  851 TTGGAAGCAT AGGGATGGGA CTTGAAGCAT ACAGATACGC TTGCAATGTG
  901 AAGTGGAITG GICTCCGAGG AGATGATCTG AATCTGATAC CAGAAGAGIC
  951 TITGGTTCCC TIAAAGCCAA AAGATTCACA GATTGCTAAG AGCTTATTGT
1001 CCTCCAAAAT ATTGCAGGAA AACTACATAG AGGAGITGTC ACTGATGGIT
1051 CAAACTGGTA AAAGAGCGGA AAITGAAGCT CTCTAITGTC ATGGTTATAA
1101 TTATCTCGGT AAATATATAG CTACCAAGAT CGIGCAAGGQ AAATACATAT
1151 AA
SEQ ID NO: 31
Arabidopsis SPO11-2; Locus# AT1G63990 Protein Sequence
    1 MEESSGLSSM KFESDQHLSY ADILLPHEAR ARIEVSVINL LRILNSPDPA
  51 ISDLSLINRK RSNSCINKGI LTDVSYIFLS TSFIKSSLIN AKTAKAFVRV
101 WKVMEICFQI LLQEKRVTQR ELFYKLLCDS PDYESSQIEV NRSVQDVVAL
151 LRCSRYSLGI MASSRGLVAG RLFLQEPGKE AVDCSACGSS GFAITGDINL
201 LDNTIMRIDA RYIIIVEKHA IFHRLVEDRV FNHIPCVFIT AKGYPDIATR
251 FFLHRMSTIF PDLPILVLVD WNPAGLAILC TEKEGSIGMG LEAYRYACNV
301 KWIGIRGDDL NLIPEESLVP LKPKDSQIAK SLLSSKILQE NYIEELSLMV
351 QTGKRAEIEA LYCHGYNYLG KYIATKIVQG KYI
SEQ ID NO: 32
RICE OsREC8; Locus# LOC_Os05g50410 CDS Sequence
>LOC_Os05g50410.1
ATGTTCTACTCGCACCAGCTCCTCGCGCGGAAGGCTCCGCTCGGCCAGATATGGATGGCG
GCGACGCTTCACTCGAAGATCAACCGGAAGCGGCTTGACAAGCTCGACATCATCAAAATC
TGTGAGGAGATTTTGAACCCGTCGGTACCCATGGCACTAAGGCTCTCCGGAATTCTCATG
GGTGGTGTGGCGATCGTGTACGAGAGGAAGGTGAAGGCTCTGTATGATGATGTGTCTCGG
TTTCTGATTGAGATCAACGAGGCATGGCGGGTCAAGCCAGTCGCAGACCCCACCGTACTT
CCCAAGGGCAAAACCCAAGCCAAGTATGAAGCAGTAACACTGCCAGAGAATATCATGGAT
ATGGATGTGGAGCAGCCCATGCTTTTCTCAGAGGCTGATACTACAAGGTTCCGGGGAATG
CGTTTGGAGGATTTGGATGACCAATACATTAATGTCAACCTAGACGATGATGACTTCTCG
CGCGCTGAGAATCATCACCAAGCTGATGCAGAAAATATCACCCTGGCTGATAATTTCGGG
TCTGGGCTTGGAGAGACTGATGTGTTCAATCGTTTTGAGAGATTCGACATAACAGATGAT
GATGCAACTTTCAATGTCACTCCTGATGGACACCCACAGGTTCCAAGTAATCTGGTTCCT
TCTCCACCTAGGCAGGAAGACTCTCCTCAGCAACAAGAAAACCATCATGCTGCCTCATCC
CCTCTTCACGAAGAAGCTCAACAAGGGGGGGCATCTGTAAAAAATGAGCAAGAGCAGCAG
AAGATGAAGGGTCAGCAACCTGCTAAATCATCAAAGAGAAAAAAACGTAGGAAAGATGAT
GAGGTGATGATGGATAACGACCAGATAATGATCCCAGGAAATGTATATCAAACATGGCTG
AAGGATCCATCAAGCCTCATTACCAAAAGGCACAGAATCAACAGTAAAGTTAATCTTATT
CGGTCAATCAAGATAAGAGACCTCATGGACTTGCCCCTCGTTTCTCTAATATCTTCCTTG
GAGAAGTCACCCTTAGAATTTTATTATCCTAAGGAACTTATGCAGCTTTGGAAGGAATGT
ACTGAAGTCAAGTCCCCAAAAGCTCCATCTTCAGGAGGGCAGCAGTCATCATCACCAGAA
CAACAGCAAAGAAACTTGCCTCCTCAGGCATTTCCAACCCAGCCTCAGGTTGATAATGAC
AGGGAAATGGGATTTCACCCAGTGGACTTTGCAGATGACATCGAAAAACTCCGAGGAAAC
ACTAGTGGGGAATATGGAAGAGATTATGATGCTTTTCACAGTGATCATAGTGTTACTCCT
GGAAGTCCTGGGCTAAGTCGCAGGTCTGCTTCAAGCTCTGGTGGCTCTGGACGGGGATTT
ACGCAGTTGGATCCAGAAGTACAGTTGCCATCCGGAAGGTCCAAGAGGCAGCATTCATCT
GGAAAAAGCTTTGGGAACCTCGATCCAGTTGAAGAAGAATTCCCATTCGAGCAAGAACTT
AGAGATTTCAAGATGAGAAGGCTTTCAGATGTTGGGCCAACTCCAGACCTGCTGGAAGAA
ATCGAACCTACTCAAACCCCATATGAAAAGAAATCCAATCCTATCGACCAGGTCACACAA
TCAATCCACTCGTACCTCAAGCTACACTTTGACACCCCAGGGGCCTCACAGTCTGAATCA
TTAAGTCAGCTAGCACATGGGATGACTACAGCAAAGGCTGCCCGACTCTTCTATCAAGCA
TGCGTTTTAGCAACTCATGATTTTATCAAGGTTAACCAGCTGGAACCATACGGAGACATC
TTGATCTCGAGGGGACCAAAGATGTGA
SEQ ID NO: 33
RICE OsREC8; Locus# LOC_Os05g50410Protein Sequence
MFYSHQLLARKAPLGQIWMAATLHSKINRKRLDKLDIIKICEEILNPSVPMALRLSGILM
GGVAIVYERKVKALYDDVSRELIEINEAWRVKPVADPTVLPKGKTQAKYEAVTLPENIMD
MDVEQPMLFSEADTTRFRGMRLEDLDDQYINVNLDDDDESRAENHHQADAENITLADNFG
SGLGETDVFNRFERFDITDDDATENVTPDGHPQVPSNLVPSPPRQEDSPQQQENHHAASS
PLHEEAQQGGASVKNEQEQQKMKGQQPAKSSKRKKRRKDDEVMMDNDQIMIPGNVYQTWL
KDPSSLITKRHRINSKVNLIRSIKIRDLMDLPLVSLISSLEKSPLEFYYPKELMQLWKEC
TEVKSPKAPSSGGQQSSSPEQQQRNLPPQAFPTQPQVDNDREMGEHPVDFADDIEKLRGN
TSGEYGRDYDAFHSDHSVTPGSPGLSRRSASSSGGSGRGFTQLDPEVQLPSGRSKRQHSS
GKSFGNLDPVEEEFPFEQELRDFKMRRLSDVGPTPDLLEEIEPTQTPYEKKSNPIDQVTQ
SIHSYLKLHFDTPGASQSESLSQLAHGMTTAKAARLFYQACVLATHDFIKVNQLEPYGDI
LISRGPKM*
SEQ ID NO: 34
Arabidopsis REC8; Locus# AT5G05490 CDS Sequence
>AT5G05490.1

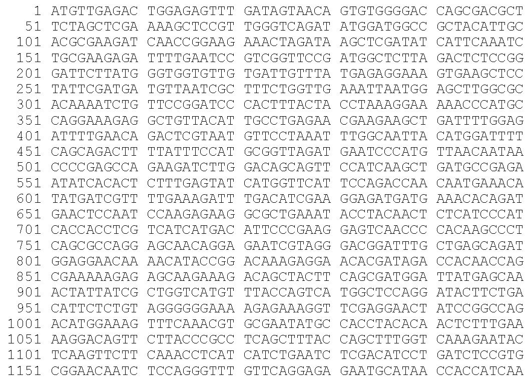

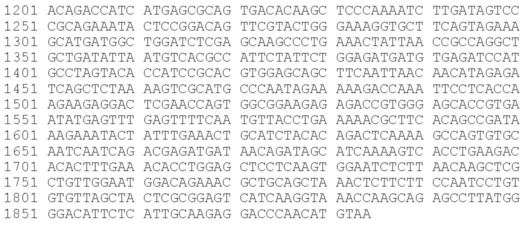


SEQ ID NO: 35
Arabidopsis REC8; Locus# AT5G05490 Protein Sequence
    1 MLRLESLIVT VWGPATLLAR KAPLGQIWMA ATLHAKINRK KLDKLDIIQI
  51 CEEIINPSVP MALRLSGILM GGVVIVYERK VKLLFDDVNR FLVEINGAWR
101 TKSVPDPTLL PKGKTHARKE AVTIPENEEA DEGDFEQTRN VPKEGNYMDE
151 QQTFISMRLD ESHVNNNPEP EDLGQQFHQA DAENITAFEY HGSFQINNET
201 YDRFEREDIE GDDETQMNSN PREGAEIPTT LIPSPPRHHD IPEGVNPTSP
251 QRQEQQENRR DGEAEQMEEQ NIPDKEEHDR PQPAKKRARK TAISAMDYEQ
301 TIIAGHVYQS WLQDISDILC RGEKRKVRGT IRPDMESEKR ANMPPTQLFE
351 KDSSYPPQLY QLWSKNTQVL QTSSSESRAP DIRAEQSPGF VQERMHNHHQ
401 TDHHERSDTS SQNLDSPAEI LRTVRTGKGA SVESMMAGSR ASPETINRQA
451 ADINVTPFYS GDDVRSMPST PSARGAASIN NIEISSKSRM PNRKRPNSSP
501 RRGLEPVAEE RPWEHREYEF EFSMLPEKRE TADKEILFET ASTQTQKPVC
551 NQSDEMITDS IKSELKTHFE TPGAPQVESL NKLAVGMDRN AAAKLFFQSC
601 VLATRGVIKV NQAEPYGDIL TARGPNM
SEQ ID NO: 36
Arabidopsis Gene Name: TAM1 (TARDY ASYNCHRONOUS MEIOSIS1); Locus
#AT1G77390 CDS Sequence
>AT1G77390.1

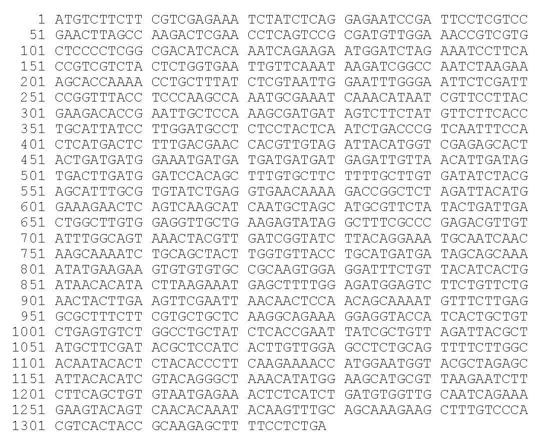

SEQ ID NO: 37
Arabidopsis TAM1 Protein Sequence
>AT1G77390.1
    1 MSSSSRNESQ ENPIPRPNLA KTRISERDVG NRRAPLGDIT NQKNGSRNPS
  51 PSSTLVNCSN KIGQSKKAPK PALSRNWNLG ILDSGLPPKP NAKSNIIVPY
101 EDTELLQSDD SLLCSSPALS LDASPTQSDP SISTHDSLTN HVVDYMVEST
151 TDDGNDDDDD EIVNIDSDEM DPQLCASFAC DIYEHLRVSE VNKRPALDYM
201 ERTQSSINAS MRSILIDWLV EVAEEYRISP ETLYLAVNYV DRYLTGNAIN
251 KQNLQLLGVT CMMIAAKYEE VCVPQVEDFC YITDNTYLRN ELLEMESSVL
301 NYLKFELTTP TAKCFLRREL RAAQGRKEVP SLLSECLACY LTELSLLDYA
351 MLRYAPSLVA ASAVELAQYT LHPSRKPWNA TLEHYTSYRA KHMEACVKNE
401 LQLCNEKLSS DVVAIRKKYS QHKYKFAAKK LCPTSLPQEL FL
SEQ ID NO: 38
cyclin-A1; LOCUS# LOC_Os12g20324 CDS Sequence
>LOC_Os12g20324.3
ATGTCGACGTGCGACTCAATGAAAAGCCCAGACTTTGAGTATATTGATAATGGGGATTCC
TCCTCAGTTCTAGGTTCCTTGCAGCGAAGAGCAAACGAGAACCTGCGTATCTCAGAGGAT
AGAGATGTTGAAGAAACTAAGTGGAAGAAGGATGCTCCTTCCCCAATGGAAATCGACCAA
ATTTGTGATGTTGACAATAACTACGAGGATCCGCAGTTGTGTGCTACTCTTGCTTCTGAT
ATCTACATGCACTTGCGCGAGGCCGAGACCAGGAAACATCCATCAACCGATTTTATGGAA
ACACTCCAAAAGGATGTAAACCCAAGCATGAGAGCGATCCTGATAGACTGGCTTGTGGAA
GTCGCTGAAGAATATCGTCTTGTTCCTGATACATTATACCTGACAGTTAACTACATTGAC
CGTTATCTTTCTGGCAATGAGATCAATCGTCAAAGACTGCAATTACTTGGAGTTGCTTGT
ATGCTTATTGCTGCAAAATACAAGGAGATATGTGCACCTCAAGTAGAAGAATTCTGCTAT
ATAACTGACAACACATACTTCAGAGATGAGGTTTTGGAAATGGAAGCTTCTGTCCTGAAT
TACCTGAAGTTTGAAATGACTGCACCTACAGCAAAATGCTTTTTGAGGAGATTTGTCCGT
GTTGCACAAGTATCTGATGAGGATCCAGCATTGCATCTTGAGTTCCTAGCCAATTATGTT
GCTGAGCTATCACTGCTGGAGTACAATCTACTTTCTTACCCTCCTTCACTAGTAGCGGCA
TCAGCTATTTTCCTGGCCAAATTCATACTGCAGCCAGCAAAGCACCCTTGGAATTCCACC
CTTGCTCACTACACACAATACAAGTCGTCAGAGTTAAGCGACTGCGTTAAGGCATTGCAC
CGCCTTTTCTGTGTTGGTCCTGGGAGTAACCTTCCTGCAATCAGGGAGAAGTATACCCAA
CATAAGTACAAATTTGTGGCGAAGAAGCCCTGCCCACCCTCAATACCGACCGAATTCTTT
CGCGACTCAACATGCTGA
SEQ ID NO: 39
cyclin-A1; LOCUS# LOC_Os12g20324 Protein Sequence
MSTCDSMKSPDFEYIDNGDSSSVLGSLQRRANENLRISEDRDVEETKWKKDAPSPMEIDQ
ICDVDNNYEDPQLCATLASDIYMHLREAETRKHPSTDEMETLQKDVNPSMRAILIDWLVE
VAEEYRLVPDTLYLTVNYIDRYLSGNEINRQRLQLLGVACMLIAAKYKEICAPQVEEFCY
ITDNTYFRDEVLEMEASVLNYLKFEMTAPTAKCFLRRFVRVAQVSDEDPALHLEFLANYV
AELSLLEYNLLSYPPSLVAASAIFLAKFILQPAKHPWNSTLAHYTQYKSSELSDCVKALH
RLFCVGPGSNLPAIREKYTQHKYKFVAKKPCPPSIPTEFFRDSTC
SEQ ID NO: 40
cyclin-A1; LOCUS#LOC_Os05g14730
CDS Sequence
>LOC_Os05g14730.1
ATGTCTAAGGAAGATGCTATGTCAACTGGTGATTCAACGGAAAGCCTTGATATTGATTGC
CTTGATGATGGGGACTCCGAAGTGGTATCTTCCTTGCAACATTTGGCAGATGATAAGCTT
CATATTTCTGACAACAGGGATGTTGCAGGTGTGGCATCCAAATGGACGAAGCATGGTTGT
AATTCAGTAGAAATTGATTATATCGTCGACATTGACAACAACCATGAGGATCCACAGCTG
TGTGCAACTCTTGCTTTTGACATTTACAAGCACTTGCGAGTGGCTGAGACCAAGAAAAGG
CCTTCAACAGATTTTGTGGAAACCATTCAGAAGAACATTGACACAAGCATGAGGGCAGTG
TTAATAGACTGGCTTGTGGAAGTCACAGAAGAATATCGGCTTGTACCTGAAACCTTATAC
CTCACAGTCAATTACATTGACCGGTATCTCTCGAGCAAGGTGATCAATCGGCGGAAAATG
CAATTACTTGGTGTCGCTTGCCTGCTTATAGCTTCTAAGTATGAAGAGATATGCCCACCC
CAAGTAGAAGAGCTCTGCTATATTTCTGACAATACATACACTAAGGATGAGGTTTTGAAA
ATGGAAGCTTCTGTCCTGAAATACTTGAAGTTTGAGATGACTGCACCTACAACAAAATGC
TTTTTGAGGAGATTTCTACGAGCTGCTCAAGTATGCCATGAGGCTCCAGTTTTGCATCTT
GAGTTCCTAGCTAATTACATTGCGGAGCTATCACTTCTGGAGTACAGCTTAATTTGCTAT
GTACCGTCACTTATAGCTGCGTCTTCTATTTTCTTGGCGAAGTTTATCCTTAAGCCAACA
GAGAATCCTTGGAATTCAACACTTTCATTCTACACACAATACAAACCATCCGACCTATGC
AATTGTGCAAAAGGACTACACCGGCTTTTCTTGGTTGGCCCTGGAGGCAACCTTCGAGCA
GTTAGAGAAAAATACAGTCAACACAAGTACAAATTCGTAGCAAAGAAGTACTCTCCACCA
TCAATTCCAGCAGAGTTTTTCGAAGATCCAAGCAGCTACAAGCCTGATTAA
SEQ ID NO: 41
cyclin-A1; LOCUS#LOC_Os05g14730Protein Sequence
MSKEDAMSTGDSTESLDIDCLDDGDSEVVSSLQHLADDKLHISDNRDVAGVASKWTKHGC
NSVEIDYIVDIDNNHEDPQLCATLAFDIYKHLRVAETKKRPSTDFVETIQKNIDTSMRAV
LIDWLVEVTEEYRLVPETLYLTVNYIDRYLSSKVINRRKMQLLGVACLLIASKYEEICPP
QVEELCYISDNTYTKDEVLKMEASVLKYLKFEMTAPTTKCFLRRFLRAAQVCHEAPVLHL
EFLANYIAELSLLEYSLICYVPSLIAASSIFLAKFILKPTENPWNSTLSFYTQYKPSDLC
NCAKGLHRLFLVGPGGNLRAVREKYSQHKYKFVAKKYSPPSIPAEFFEDPSSYKPD
SEQ ID NO: 42
cyclin-A1; Locus# LOC_Os01g13229 CDS Sequence
>LOC_Os01g13229.1
ATGGCCTTGGTGTGTGCGGAATGCCGTCTTGTTGACATCGTCCGGGTGCATGCGAACCTG
ATGGTACCCGAAATGGAGATCCAGCTTGGAGAAGAAGTGGGCGTCGCAAAGTTCATCAAG
CAATTCGTCGAGAACCAAAATGGGAAACATGTCCTTGGCAGTCACCGTGTTGAGGGCTCG
GTAGTCCACGTAGAAGCGCCATGGCTTGTGGACCAACAGAACCGACGACGAGAATGGCGA
CGTGTTGGGGCAGATGGAGAGCTTGTCAGCGCATTATTTCTCCAATTCGTCGTTTTGAAG
CTGTGGGTAGCGATACGGTCGAACCACTACCGGGGGCTTGCCGGGCTTGAGGTGGATGTG
ATGATTGATGGATCGGGTCGGTGGGAGGCCGGTTGGAGCGCGAAGAGGTCGTCAAATTCC
GCCAGCAAGGACGGAAGGAGATCGTCGGCGTGCAGTGCAGCGCAGCTGAGGTCGAGGGCG
AAGCAGTCGACGTCGAACACCTCGGTGGCCACCTACAGGCGAAGCCCGTGCACGACACCC
GCGCCGCGGATGCGATCGTTGTCAGCCACCACCACGCGGAGACCTTGGCGTACCGGTTGG
AGAGGTAGGCCGATGCGCTGGGCCAGCTTGGTGGTGATGAAATTGTGTGTTGAAGCCGGA
GTCAACAAGGGCCATCACCTGCTCGGCCGCGATACGCGCCACGAGGCACATCGTGCTAGT
GCCATTGACACCGAAAATCGCGTTGAGCGAGATGTGTGGTTCATCGGTGTCGTCTGTGTC
GTCCCATTCGCTGAAATTAGCCGTGGTGTCGTCGTACTCAAGAGGCCTTGTTTACAGCGC
TCTGGTAACTCTGCTGGCGAGAGACAACGAAATTGGTGTCCTGGCACGAGCCAAGGCCAT
GGCCTCCTGGAAATCCTCGGGCTGTTGGAGCTCGACATCGATGCAGATGTCGTCGGTGAG
CCCCGCTGTGAAGAGCTGAACTTGTTGGCGGTCGGTGAGGAGGTCGGACGTGCGGCTACC
CAACGCCAAAAGGCGCTGCTGGTACGTCGTCACCGTGCCAACCTGAGTGAGATGCTTGAG
TTCACCCAGGGAATTGTTGCGGATCGGCGGTCCATACTGACCGTAACAGAGCTGCTTGAA
CGTATCCCAATCCGGAGGACCCATGTGCTGCTCATAATGGAAGTACCATTCCTGTGCAGC
TCAAGTGAGATGACCAGGAAACGTCCATCAACTGATTTTATGGAAACAATCCAAAAGGAT
GTAAACCCAAGCATGAGAGCGATCCTGATAGACTGGCTTGTGGAAGTCGCTGAAGAATAT
CGTCTTGTTCCTGATACATTATACCTGACAGTTAACTACATTGATCGTTATCTTTCTGGC
AATGAGATCAATCGTCAAAGACTGCAATTACTTGGAGTTGCTTGTATGCTTATTGCTGCA
AAATACGAGGAGATATGTGCACCTCAAGTAGAAGAATTCTGCTATATAACTGACAACACA
TACTTCAGAGATGAGTGCTGGAATGAATCGAACTCTAATAACTCTCTTATTGCCTACAAC
AGGAGATTTGTCCGTGTTGCACAAGTATCGGATGAGCTTTTCATCGTGCAGGATCCAGCA
TTGCATCTTGAGTTCCTAGCCAATTATGTTGCTGAGCTATCACTGCTGGAGTACAATCTA
CTTTCTTACCCTCCTTCACTAGTAGCGGCATCGGCTATTTTCTTGGCCAAATTCATACTG
CAGCCAACAAAGCACCCTTGGAATTCCACCCTTGCTCACTACACACAATACAAGTCGTCA
GAGTTAAGCGACTGTGTAAAGGCATTGCACCGCCTTTTTAGCGTTGGTCCCGGGAGTAAC
CTTCCTGCAATCAGGGAGAAGTATACCCAACATAAGATACTGCATGCAGCTGATGTGATC
GACTTGAACATGGCAAATGCATTTAAGAATGTGAAAATATTATGTCAATGTCCCTGTCAA
TGCAACCTTCTTGAAGAAGTCATGCTCAAGCTATTTCCATACTGGAAGCTAAGCACAGCT
GTTTAG
SEQ ID NO: 43
cyclin-A1; Locus# LOC_Os01g13229 Protein Sequence
MALVCAECRLVDIVRVHANLMVPEMEIQLGEEVGVAKFIKQFVENQNGKHVLGSHRVEGS
VVHVEAPWLVDQQNRRREWRRVGADGELVSALFLQFVVLKLWVAIRSNHYRGLAGLEVDV
MIDGSGRWEAGWSAKRSSNSASKDGRRSSACSAAQLRSRAKQSTSNTSVATYRRSPCTTP
APRMRSLSATTTRRPWRTGWRGRPMRWASLVVMKLCVEAGVNKGHHLLGRDTRHEAHRAS
AIDTENRVERDVWFIGVVCVVPFAEISRGVVVLKRPCLQRSGNSAGERQRNWCPGTSQGH
GLLEILGLLELDIDADVVGEPRCEELNLLAVGEEVGRAATQRQKALLVRRHRANLSEMLE
FTQGIVADRRSILTVTELLERIPIRRTHVLLIMEVPFLCSSSEMTRKRPSTDEMETIQKD
VNPSMRAILIDWLVEVAEEYRLVPDTLYLTVNYIDRYLSGNEINRQRLQLLGVACMLIAA
KYEEICAPQVEEFCYITDNTYERDECWNESNSNNSLIAYNRRFVRVAQVSDELFIVQDPA
LHLEFLANYVAELSLLEYNLLSYPPSLVAASAIFLAKFILQPTKHPWNSTLAHYTQYKSS
ELSDCVKALHRLFSVGPGSNLPAIREKYTQHKILHAADVIDLNMANAFKNVKILCQCPCQ
CNLLEEVMLKLFPYWKLSTAV*
SEQ ID NO: 44
cyclin-A1; Locus # LOC_Os12g31810 CDS Sequence
>LOC_Os12g31810.1
ATGGCTGGAAGGAAGGAAAATCCGGTGCTTACTGCTTGCCAAGCACCCAGTGGTCGAATC
ACACGAGCTCAAGCTGCTGCAAATCGTGGACGGTTTGGGTTTGCTCCCTCCGTATCACTA
CCCGCAAGAACTGAACGAAAGCAGACAGCAAAAGGAAAGACAAAAAGGGGAGCTTTGGAT
GAAATCACTAGTGCAAGTACTGCAACTTCAGCTCCTCAGCCTAAACGGCGCACAGTGCTC
AAGGATGTAACCAACATCGGCTGTGCCAACTCATCCAAAAATTGCACCACCACGAGCAAG
CTGCAGCAAAAGTCAAAGCCCACCCAAAGGGTGAAACAAATCCCGAGCAAAAAGCAGTGT
GCAAAGAAGGTTCCTAAGCTACCCCCTCCGGCTGTTGCTGGAACTTCATTTGTGATTGAT
TCTAAAAGTTCTGAAGAAACTCAAAAGGTGGAGCTTTTGGCAAAAGCAGAGGAACCCACA
AATTTGTTTGAAAACGAGGGGTTACTGTCATTGCAGAATATTGAGCGAAACAGGGACAGT
AATTGCCATGAGGCATTCTTTGAGGCAAGAAACGCCATGGATAAACATGAACTCGCTGAC
TCCAAGCCTGGTGACTCTAGTGGTTTAGGTTTTATAGATATTGACAATGATAATGGAAAT
CCTCAAATGTGTGCTTCCTATGCTTCAGAGATATACACAAATCTGATGGCCTCTGAGCTT
ATCAGAAGACCCAGGTCAAATTACATGGAGGCTTTGCAACGTGACATCACAAAGGGCATG
CGAGGCATTCTCATTGATTGGCTTGTTGAGGTTTCTGAAGAATATAAGCTTGTGCCAGAC
ACACTCTACCTAACCATTAATCTTATTGACCGATTTCTTTCTCAACATTATATTGAAAGA
CAGAAACTCCAACTTCTTGGAATAACAAGCATGCTGATTGCCTCGAAATATGAAGAGATA
TGTGCTCCTCGTGTTGAAGAATTTTGTTTCATAACTGACAATACATACACAAAAGCTGAG
GTGCTGAAAATGGAGGGCCTGGTGCTTAATGATATGGGGTTTCATCTATCTGTTCCAACA
ACAAAAACATTTCTCAGGAGATTCCTTAGAGCCGCACAGGCTTCTCGTAATGTTCCTTCA
ATTACCTTGGGATATCTGGCCAATTATCTTGCAGAGCTGACCCTGATCGATTACAGTTTC
CTCAAATTTCTTCCTTCAGTGGTGGCAGCATCTGCAGTCTTTCTTGCAAGATGGACACTT
GACCAATCTGACATTCCATGGAATCATACTCTTGAGCACTACACTTCTTACAAAAGCTCT
GATATTCAAATATGTGTCTGTGCTCTACGGGAACTGCAGCATAACACCAGTAATTGCCCT
CTCAATGCTATACGTGAAAAGTATAGGCAACAAAAGTTTGAGTGTGTAGCCAACCTGACA
TCACCGGAGCTGGGGCAGTCACTCTTCAGCTGA
SEQ ID NO: 45
cyclin-A1; Locus # LOC_Os12g31810 Protein Sequence
MAGRKENPVLTACQAPSGRITRAQAAANRGRFGFAPSVSLPARTERKQTAKGKTKRGALD
EITSASTATSAPQPKRRTVLKDVTNIGCANSSKNCTTTSKLQQKSKPTQRVKQIPSKKQC
AKKVPKLPPPAVAGTSFVIDSKSSEETQKVELLAKAEEPTNLFENEGLLSLQNIERNRDS
NCHEAFFEARNAMDKHELADSKPGDSSGLGFIDIDNDNGNPQMCASYASEIYTNLMASEL
IRRPRSNYMEALQRDITKGMRGILIDWLVEVSEEYKLVPDTLYLTINLIDRELSQHYIER
QKLQLLGITSMLIASKYEEICAPRVEEFCFITDNTYTKAEVLKMEGLVLNDMGFHLSVPT
TKTFLRRELRAAQASRNVPSITLGYLANYLAELTLIDYSFLKFLPSVVAASAVFLARWTL
DQSDIPWNHTLEHYTSYKSSDIQICVCALRELQHNTSNCPLNAIREKYRQQKFECVANLT
SPELGQSLFS*
SEQ ID NO: 46
cyclin-A1; Locus # LOC_Os01g13260 CDS Sequence
>LOC_Os01g13260.1
ATGTCGAGCAACCTAGCAGCCTCCCGCCGCTCGTCGTCGTCGTCCTCGGTGGCGGCGGCG
GCGGCGGCGAAGCGACCCGCGGTGGGGGAGGGAGGAGGAGGAGGAGGAGGGAAGGCGGCA
GCGGGCGCCGCCGCGGCAAAGAAGCGCGTGGCGCTTAGCAACATCAGCAACGTCGCCGCT
GGTGGTGGCGCCCCAGGGAAGGCCGGCAATGCGAAGTTGAATTTAGCTGCCTCAGCTGCA
CCAGTGAAGAAGGGATCTTTGGCCAGTGGCCGCAATGTGGGCACGAATCGGGCCTCGGCG
GTGAAATCGGCTTCCGCCAAGCCGGCTCCGGCCATATCCCGCCATGAGAGCGCCACACAG
AAGGAGTCTGTTCTTCCTCCTAAAGTGCCTAGCATTGTGCCGACTGCTGCACTGGCACCT
GTCACTGTACCCTGCAGCAGCTTCGTCTCCCCTATGCATTCAGGAGATTCAGTTTCGGTT
GACGAGACGATGTCGACGTGTGACTCAATGAAAAGCCCAGAATTTGAGTACATTGATAAT
GGGGATTCCTCCTCAGTTCTAGGTTCCTTGCAGCGAAGAGCAAACGAAAACCTGCGTATC
TCAGAGGATAGAGATGTCGAAGAAACTAAGTGGAAGAAGGATGCTCCTTCCCCAATGGAA
ATCGACCAAATTTGTGATGTTGACAATAACTACGAGGATCCGCAGTTGTGTGCTACTCTT
GCTTCTGATATCTACATGCACTTGCGCGAGGCTGAGACCAGGAAACGTCCATCAACTGAT
TTTATGGAAACAATCCAAAAGGATGTAAACCCAAGCATGAGAGCGATCCTGATAGACTGG
CTTGTGGAAGTCGCTGAAGAATATCGTCTTGTTCCTGATACATTATACCTGACAGTTAAC
TACATTGATCGTTATCTTTCTGGCAATGAGATCAATCGTCAAAGACTGCAATTACTTGGA
GTTGCTTGTATGCTTATTGCTGCAAAATACGAGGAGATATGTGCACCTCAAGTAGAAGAA
TTCTGCTATATAACTGACAACACATACTTCAGAGATGAGGTTTTGGAAATGGAAGCTTCT
GTCCTGAATTACCTGAAGTTTGAAGTGACTGCACCTACAGCAAAATGCTTTTTGAGGAGA
TTTGTCCGTGTTGCACAAGTATCGGATGAGGATCCAGCATTGCATCTTGAGTTCCTAGCC
AATTATGTTGCTGAGCTATCACTGCTGGAGTACAATCTACTTTCTTACCCTCCTTCACTA
GTAGCGGCATCGGCTATTTTCTTGGCCAAATTCATACTGCAGCCAACAAAGCACCCTTGG
AATTCCACCCTTGCTCACTACACACAATACAAGTCGTCAGAGTTAAGCGACTGTGTAAAG
GCATTGCACCGCCTTTTTAGCGTTGGTCCCGGGAGTAACCTTCCTGCAATCAGGGAGAAG
TATACCCAACATAAGTACAAATTTGTGGCGAAGAAGCCCTGCCCACCCTCAATACCGACC
GAATTCTTTCGCGACGCAACATGCTGA
SEQ ID NO: 47
cyclin-A1; Locus # LOC_Os01g13260 Protein Sequence
MSSNLAASRRSSSSSSVAAAAAAKRPAVGEGGGGGGGKAAAGAAAAKKRVALSNISNVAA
GGGAPGKAGNAKLNLAASAAPVKKGSLASGRNVGTNRASAVKSASAKPAPAISRHESATQ
KESVLPPKVPSIVPTAALAPVTVPCSSFVSPMHSGDSVSVDETMSTCDSMKSPEFEYIDN
GDSSSVLGSLQRRANENLRISEDRDVEETKWKKDAPSPMEIDQICDVDNNYEDPQLCATL
ASDIYMHLREAETRKRPSTDEMETIQKDVNPSMRAILIDWLVEVAEEYRLVPDTLYLTVN
YIDRYLSGNEINRQRLQLLGVACMLIAAKYEEICAPQVEEFCYITDNTYERDEVLEMEAS
VLNYLKFEVTAPTAKCFLRRFVRVAQVSDEDPALHLEFLANYVAELSLLEYNLLSYPPSL
VAASAIFLAKFILQPTKHPWNSTLAHYTQYKSSELSDCVKALHRLFSVGPGSNLPAIREK
YTQHKYKFVAKKPCPPSIPTEFFRDATC*
SEQ ID NO: 48
Cyclin-A3; Locus # LOC_Os12g39210 CDS Sequence
>LOC_Os12g39210.1
ATGGCTGACAAGGAGAACTCCACCCCGGCCTCCGCGGCGCGGCTCACCCGCTCGTCTGCG
GCGGCTGGGGCGCAGGCCAAGCGTTCGGCCGCCGCGGGCGTCGCCGACGGTGGCGCGCCG
CCGGCGAAGAGGAAGCGCGTCGCGCTCAGCGACCTCCCGACCCTCTCCAACGCCGTCGTC
GTCGCCCCCCGCCAGCCGCACCACCCCGTCGTCATCAAGCCGTCGTCCAAGCAGCCCGAG
CCCGCCGCGGAGGCGGCGGCGCCCAGCGGCGGCGGCGGCGGCTCCCCCGTGTCATCCGCG
TCGACGTCGACGGCGTCGCCCTCCTCCGGTTGGGACCCGCAGTACGCCTCCGACATCTAC
ACCTACCTCCGATCCATGGAGGTGGAGGCGCGGAGGCAGTCGGCGGCGGACTACATCGAG
GCGGTGCAGGTGGACGTGACGGCGAACATGCGGGCCATCCTCGTGGACTGGCTGGTGGAG
GTCGCCGACGAGTACAAGCTCGTCGCCGACACGCTCTACCTCGCCGTCTCCTACCTCGAC
CGCTACCTCTCCGCCCACCCGCTCAGGCGCAACAGGCTGCAGCTCCTCGGCGTCGGCGCC
ATGCTCATCGCTGCGAAGTACGAGGAGATTAGCCCTCCTCATGTGGAGGATTTCTGCTAC
ATCACTGATAATACGTACACTAGGCAGGAGGTTGTCAAGATGGAGAGCGACATACTCAAG
CTTCTCGAGTTCGAGATGGGCAATCCTACCATCAAGACATTCCTCAGGCGGTTCACGAGA
TCTTGCCAGGAAGACAAAAAGCGCTCCAGCTTGTTATTGGAGTTCATGGGGAGTTATCTT
GCTGAGCTTAGTCTACTTGACTACGGCTGTCTCCGGTTCTTGCCATCGGTGGTTGCTGCC
TCAGTGGTGTTTGTTGCTAAACTGAACATTGATCCGTACACCAATCCTTGGAGCAAGAAG
ATGCAGAAGTTGACAGGATACAAGGTGTCTGAACTGAAGGATTGCATCTTGGCCATTCAT
GACTTGCAGCTCAGAAAAAAATGTTCAAACTTAACTGCAATTCGCGACAAGTACAAGCAA
CACAAGTTCAAGTGTGTCTCAACATTGCTTCCCCCTGTTGATATCCCTGCGTCATACCTC
CAAGATTTAACAGAGTAG
SEQ ID NO:49
Cyclin-A3; Locus # LOC_Os12g39210 Protein Sequence
MADKENSTPASAARLTRSSAAAGAQAKRSAAAGVADGGAPPAKRKRVALSDLPTLSNAVV
VAPRQPHHPVVIKPSSKQPEPAAEAAAPSGGGGGSPVSSASTSTASPSSGWDPQYASDIY
TYLRSMEVEARRQSAADYIEAVQVDVTANMRAILVDWLVEVADEYKLVADTLYLAVSYLD
RYLSAHPLRRNRLQLLGVGAMLIAAKYEEISPPHVEDFCYITDNTYTRQEVVKMESDILK
LLEFEMGNPTIKTFLRRFTRSCQEDKKRSSLLLEFMGSYLAELSLLDYGCLRFLPSVVAA
SVVFVAKLNIDPYTNPWSKKMQKLTGYKVSELKDCILAIHDLQLRKKCSNLTAIRDKYKQ
HKFKCVSTLLPPVDIPASYLQDLTE*
SEQ ID NO:50
Cyclin-A3; Locus# LOC_Os03g41100 CDS sequence
>LOC_Os03g41100.1
ATGGCCGGCAAGGAGAACGCCGCGGCGGCGCAGCCCCGCCTCACCCGCGCCGCCGCCAAG
CGCGCGGCCGCCGTCACCGCCGTGGCCGTCGCCGCCAAGCGCAAGCGCGTCGCGCTCAGC
GAGCTCCCCACGCTGTCCAACAACAACGCCGTGGTGCTCAAGCCGCAGCCGGCGCCCAGG
GGCGGCAAGAGGGCCGCCTCCCACGCCGCCGAGCCCAAGAAGCCAGCTCCGCCGCCGGCG
CCGGCGGTGGTGGTCGTGGTCGACGACGACGAGGAGGGGGAGGGGGATCCGCAGCTCTGC
GCGCCCTACGCCTCCGACATCAACTCCTACCTCCGCTCCATGGAGGTGCAAGCGAAGCGG
CGGCCGGCGGCGGACTACATCGAGACGGTGCAGGTGGACGTGACGGCCAACATGCGAGGC
ATCCTGGTCGACTGGCTCGTCGAGGTCGCCGAGGAGTACAAGCTCGTCTCCGACACGCTC
TACCTCACCGTCTCCTACATCGACCGCTTCCTCTCCGCCAAATCCATCAACCGCCAGAAG
CTGCAGCTCCTCGGCGTCTCCGCCATGCTCATCGCCTCGAAGTATGAGGAGATCAGCCCC
CCAAATGTGGAGGATTTCTGCTATATAACCGACAATACCTATATGAAACAGGAGGTTGTC
AAGATGGAGCGCGATATACTGAATGTTCTCAAGTTTGAGATGGGCAATCCTACAACCAAG
ACGTTCCTGAGGATGTTCATCAGATCTAGCCAAGAAGACGATAAGTATCCTAGCCTTCCC
TTGGAGTTCATGTGTAGCTATCTTGCCGAGCTGAGCCTGCTGGAGTACGGCTGTGTTCGG
CTCTTGCCATCCGTTGTTGCAGCCTCAGTGGTGTTTGTTGCAAGGCTAACCCTTGATTCA
GACACCAATCCTTGGAGCAAGAAGTTGCAAGAGGTGACCGGCTACAGGGCATCTGAGTTG
AAGGATTGCATTACCTGCATACATGACTTGCAGCTAAACAGGAAAGGGTCATCTCTAATG
GCTATCCGGGACAAGTACAAGCAACATAGGTTCAAGGGCGTATCAACATTGTTACCCCCT
GTTGAGATCCCTGCATCATACTTCGAAGACCTAAACGAGTAG
SEQ ID NO: 51
Cyclin-A3; Locus# LOC_Os03g41100 Protein sequence
MAGKENAAAAQPRLTRAAAKRAAAVTAVAVAAKRKRVALSELPTLSNNNAVVLKPQPAPR
GGKRAASHAAEPKKPAPPPAPAVVVVVDDDEEGEGDPQLCAPYASDINSYLRSMEVQAKR
RPAADYIETVQVDVTANMRGILVDWLVEVAEEYKLVSDTLYLTVSYIDRELSAKSINRQK
LQLLGVSAMLIASKYEEISPPNVEDFCYITDNTYMKQEVVKMERDILNVLKFEMGNPTTK
TFLRMFIRSSQEDDKYPSLPLEFMCSYLAELSLLEYGCVRLLPSVVAASVVFVARLTLDS
DTNPWSKKLQEVTGYRASELKDCITCIHDLQLNRKGSSLMAIRDKYKQHRFKGVSTLLPP
VEIPASYFEDLNE
SEQ ID NO: 52
Arabidopsis Gene Name: DYAD; Locus #AT5G51330, CDS Sequence

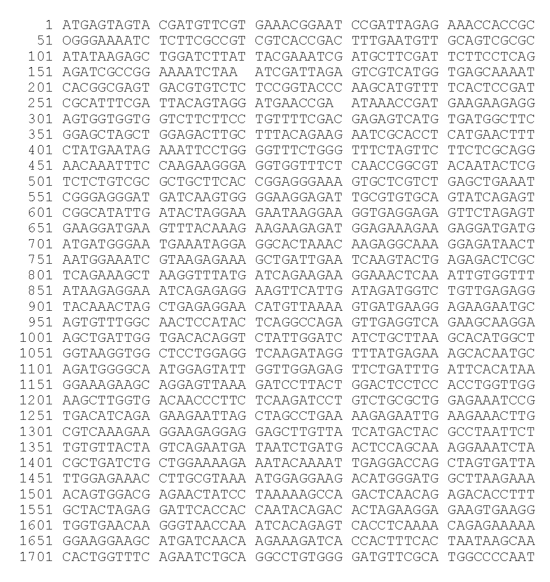

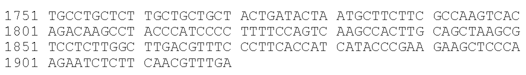


SEQ ID NO: 53
Arabidopsis DYAD; Locus #AT5G51330 Protein Sequence
    1 MSSTMFVKRN PIRETTAGKI SSPSSPTINV AVAHIRAGSY YEIDASILPQ
  51 RSPENLKSIR VVMVSKITAS DVSLRYPSMF SLRSHFDYSR MNRNKPMKKR
101 SGGGLLPVED ESHVMASELA GDLLYRRIAP HELSMNRNSW GFWVSSSSRR
151 NKFPRREVVS QPAYNTRICR AASPEGKCSS ELKSGGMIKW GRRLRVQYQS
201 RHIDTRKNKE GEESSRVKDE VYKEEEMEKE EDDDDGNEIG GTKQEAKEIT
251 NGNRKRKLIE SSTERLAQKA KVYDQKKETQ IVVYKRKSER KFIDRWSVER
301 YKLAERNMLK VMKEKNAVEG NSILRPELRS EARKLIGDTG LLDHLLKHMA
351 GKVAPGGQDR FMRKHNADGA MEYWLESSDL IHIRKEAGVK DPYWTPPPGW
401 KLGDNPSQDP VCAGEIRDIR EELASIKREL KKLASKKEEE ELVIMITPNS
451 CVTSQNDNEM TPAKEIYADL LKKKYKIEDQ LVIIGETIRK MEEDMGWLKK
501 TVDENYPKKP DSTETPLLLE DSPPIQTLEG EVKVVNKGNQ ITESPQNREK
551 GRKHDQQERS PLSLISNTGF RICRPVGMEA WPQLPALAAA TDTNASSPSH
601 RQAYPSPFPV KPLAAKRPLG LTEPFTTIPE EAPKNLENV
SEQ ID NO: 54
Rice homologs of AtDYAD SWITCH1; LOC_Os12g42820 CDS Sequence
>LOC_Os12g42820.1
ATGACCGCCGCCGCCGTCAACGCCTCGCGCGTCATGCGCCGCCGCGCCGCCGGCGAGGAC
CTGGGCGACGGCGACTTCTGGGCCGGTGGCGCCCCGCGCCTCTACGACTTCTCCCAGCAG
GAGCAGAAGCCGTTCTTGCCGGCGCCGCCGTCGCCCGCGCCCGTCCCGGCGTCGCCGCCG
TCCCCCGCGGCGGAGTCGGTGGCGCCGTGCCTCCTCACGCTGCAGTGCAGCGGCGTCGGG
TGGGGGGTCAGGAAGCGGGTCAGGTACGTCGGGAGGCACCACCACCTCGCGCGCCACCAC
GCTCCCGAACGCGCCGTGGACGCCGCGCGGGACGACGACGAGGCGAGCTCCGCCAAGGCC
AAGAACGAGAGCCCGAAGGAGGAGGCGGCGGCGGCAGAAGAAGACGACGACGTCGAACAC
AAGGTGGCGGTGCGCACCACGTCGGAGGAGAAGAAGAAGAAGAGGAGGAGGAAGCGCGGC
CGTGGCCGTGTCCGTGGCCATGGCGTCGCCAAGCGTCCCAAGAAGGAGGATGAGGAGGGG
ACGAAGCTCTCGGCTCCCAAGGCCGAGCAGCTCGAGGAGGAGGAGGAGGGCGCCGCGGTG
GCGGCGCCGAGCGGCATGATCGACCGGTGGAAGGCGACCCGGTACGCCACCGCGGAGGCG
TCGCTGCTCGCCATCATGCGCGCCCACGGCGCGCGCGCCGGGAAGCCCGTCCCGCGCGCG
GCGCTGCGGGAGGAGGCGCGCGCCCACATCGGCGACACGGGCCTCCTCGACCACCTCCTC
AGGCACATCGCCGACAAGGTGGCGCCCGGCGGCGCCGAGCGGTTCCGGCGGCGGCACAAC
GCCGGCGGCGGGCTGGAGTACTGGCTGGAGCCCGCCGAGCTCGCCGCCGTGCGGCGGAAG
GCCGGCGTGGCCGACCCGTACTGGGTGCCTCCTCCCGGATGGAAGCCAGGGGACCCCGTG
TCGCCGGAGGGCTACTTGCTGGAGGTGAGGAAGCAGGTGGAGCAGCTCGCCGTTGAGCTC
GCCGGCGTCAGAAGGCACATGGATCACCTCACTTCCAATGTGAGTCAAGTGGGCAAGGAA
ATCAAATCTGAGGCTGAGAAGTCCTACAATACATGTCAGGGTGGGGACCCACCCTACCTT
GACCGGATCTCGATCCGTGCCTTCGCCCGGAAGCTAAGCCGCAGCTCGTCCTTGAGCGGG
ACCCAGCGCCGTCCCCCGCCTGACACGGGGGACAGCCCTGTCATTCCCTCATAA
SEQ ID NO: 55
SWITCH1; LOC_Os12g42820 Protein Sequence
MTAAAVNASRVMRRRAAGEDLGDGDFWAGGAPRLYDFSQQEQKPFLPAPPSPAPVPASPP
SPAAESVAPCLLTLQCSGVGWGVRKRVRYVGRHHHLARHHAPERAVDAARDDDEASSAKA
KNESPKEEAAAAEEDDDVEHKVAVRTTSEEKKKKRRRKRGRGRVRGHGVAKRPKKEDEEG
TKLSAPKAEQLEEEEEGAAVAAPSGMIDRWKATRYATAEASLLAIMRAHGARAGKPVPRA
ALREEARAHIGDTGLLDHLLRHIADKVAPGGAERFRRRHNAGGGLEYWLEPAELAAVRRK
AGVADPYWVPPPGWKPGDPVSPEGYLLEVRKQVEQLAVELAGVRRHMDHLTSNVSQVGKE
IKSEAEKSYNTCQGGDPPYLDRISIRAFARKLSRSSSLSGTQRRPPPDTGDSPVIPS*
SEQ ID NO: 56
Locus #LOC_Os12g42830 CDS Sequence
>LOC_Os12g42830.1
ATGACCGCCGCCGCCGTCAACGCCTCGCGCGTCATGCGCCGCCGCGCCGCAGGCGAGGAC
CTGGGCGACGGCGGCGATGGCGACGGCGACTTCTGGGCCGGTGGCGCCCCCCGCCTCTAC
GACTTCTCCCAGCAGGAGCAGAAGCCGTTCTTGCCCGCGCCCGCGCCCGCGCCGCCGTCG
CCCGCGCCCGTCCCGGCGTCGCCGCCGTCCCCCGCGGCGGAGTCGGTGGCGCCGTGCCTC
CTCACGCTGCAGTGCAGCGGCGTCGGGTGGGGTGTCAGGAAGCGGGTCCGGTACGTCGGG
AGGCACCACCACCTCGCGCGCCACCACGCTCCCGAGCGCGCCGTGGACGCCGCGCGGGAC
GACGACGAGGCGAGCTCCGCCAAGGCCAAGAACGAGAGCCCGAAGGAGGAGGCAGCGGCG
GCAGAAGAAGACGACGACAACGTCGAACACAAGGTGGCGGTGCCCACCACGTCGGAGGAG
AAGAAGAGGAGGAGGAGGAGGAAGCGTGGCCGTGGCCGTGTCGGTGGCCATGGCGTCGCC
AAGCGTCCCAAGAAGGAGGAGGAGGAGGAGGAGACGAAGCTCTCGGCTCCCAAGGCCGAG
CAGCTCGAGGAGGAGGAGGGCGCCGCGGTGGCGGCGCCGAGCGGCATGATCGACCGGTGG
AAGGCGACCCGGTACGCCACCGCGGAGGCGTCGCTGCTCGCCATCATGCGCGCCCGCGGC
GCGCGCGCCGGGAAGCCCGTCCCGCGCGGGGCGCTGCGGGAGGAGGCGCGCGCCCACATT
GGCGACACGGGCCTCCTCGACCACCTCCTCAGGCACATCGCCGACAAGGTGGCGCCCGGC
GGCGCCGAGCGGTTCCGGCGGCGGCACAACGCCGGCGGCGGGCTGGAGTACTGGCTCGAG
CCCGCCGAGCTCGCCGCCGTGCGGCGGAACGCCGGCGTGGCCGACCCGTACTGGGTGCCT
CCTCCCGGATGGAAGCCAGGGGACCCCGTCTCGCCGGAGGGCTACTTGCTGGAGGTGAGG
AAGCAGGTGGAGAAGCTCGCCGTGGAGCTCGCCGGCGTCAGAAGGCACATGGATCACCTC
TCTTCCAATGTGAGTCAAGTGGGCAAGGAAATCAAATCTGAGGCTGAGAAATCCTACAAT
ACATGCCAGGAGAAGTATGCCTGTATGGAGAAAGCCAATGGCAATCTGGAAAAGCAGCTT
CTGTCCTTGGAGGAGAAGTATGAGAATGCAACACACGCAAATGGCGAGCTGAAGGAGGAG
TTGTTGTTTCTCAAGGAGAAGTTTGTGAGTGTGGTCGAGAACAACACCAGACTGGAGCAC
CAGCTGACTGCTTTATCCACTTCTTTCCTGTCTCTAAAGGAGGAACTGCTCTGGCTGGAA
AAAGAAGAAGCTGATCTGTATGTCAAGGAACCATGGGAAGACGACGATGAAAAGCAAGAA
CACGATGCCGGGAAAGAGGCGAAGGACGACGATGTCGCCGGCGTCAGTGCAGCCAACGAC
CAGCCGGACGTCGACGGCGATGGCACCACCACCACCACCACCACCAGCAGCAATGGTGGC
AGCGGGAAGAGAACATCGAGGAAGTGCAGCGTGCGCATCTCCAAGCCGCAGGGCGCGTTC
CAGTGGCCGACGCCGAGCCTGCCGTTCTCGCCGGAGCTCGCCGCGCCGCCGTCGCCGCCG
CTGACCCCGACGGCGCCCGTCGTCGCCGGCGCCGCCAACTTCGCCACCATGGACGAGCTC
TACGAGTACATGATGGCCGGCGGCCTCCCCACGCCACCGTCCACCACCAGCAACGCCGGG
AAGCTCCCCTCGCTGCCCGCCGCCACGGCCTGCGCCACGACGCCGCCGGTGAAGACGGCG
GACGCCGCCGGCGACGTGGGCACCGAGCTGGCACTGGCCACTCCCGCCTACTGA
SEQ ID NO: 57
Locus #LOC_Os12g42830 Protein sequence
MTAAAVNASRVMRRRAAGEDLGDGGDGDGDFWAGGAPRLYDFSQQEQKPFLPAPAPAPPS
PAPVPASPPSPAAESVAPCLLTLQCSGVGWGVRKRVRYVGRHHHLARHHAPERAVDAARD
DDEASSAKAKNESPKEEAAAAEEDDDNVEHKVAVPTTSEEKKRRRRRKRGRGRVGGHGVA
KRPKKEEEEEETKLSAPKAEQLEEEEGAAVAAPSGMIDRWKATRYATAEASLLAIMRARG
ARAGKPVPRGALREEARAHIGDTGLLDHLLRHIADKVAPGGAERFRRRHNAGGGLEYWLE
PAELAAVRRNAGVADPYWVPPPGWKPGDPVSPEGYLLEVRKQVEKLAVELAGVRRHMDHL
SSNVSQVGKEIKSEAEKSYNTCQEKYACMEKANGNLEKQLLSLEEKYENATHANGELKEE
LLFLKEKFVSVVENNTRLEHQLTALSTSFLSLKEELLWLEKEEADLYVKEPWEDDDEKQE
HDAGKEAKDDDVAGVSAANDQPDVDGDGTTTTTTTSSNGGSGKRTSRKCSVRISKPQGAF
QWPTPSLPFSPELAAPPSPPLTPTAPVVAGAANFATMDELYEYMMAGGLPTPPSTTSNAG
KLPSLPAATACATTPPVKTADAAGDVGTELALATPAY*
SEQ ID NO: 58
SWI1; LOCUS# LOC_Os03g44760 CDS Sequence
>LOC_Os03g44760.1
ATGGACGCGGAGATGGCGGCTCCTGCGCTTGCGGCAGCTCATCTGCTGGACTCGCCCATG
AGGCCACAGGTGAGCAGATACTACTCCAAGAAGAGGGGTAGCAGCCACAGCAGAAATGGC
AAGGATGATGCCAACCATGACGAGTCCAAGAACCAATCACCCGGCTTGCCCCTGAGCAGA
CAGAGCCTGTCCTCATCTGCCACCCACACCTACCACACCGGAGGGTTCTACGAGATCGAC
CACGAGAAGCTTCCCCCCAAATCCCCAATTCATCTCAAGTCCATACGCGTGGTAAAGGTG
AGCGGCTACACAAGCCTGGACGTCACAGTGAGCTTCCCGTCCCTCCTGGCGCTGCGAAGC
TTCTTCTCCTCCTCCCCACGGTCGTGCACTGGGCCGGAGCTCGACGAGCGCTTCGTCATG
AGCAGCAACCACGCGGCCCGCATCCTGCGCCGTCGGGTGGCCGAGGAGGAGCTCGCGGGC
GACGTGATGCACCAGGACAGCTTCTGGCTCGTCAAGCCCTGCCTCTATGACTTCTCCGCG
TCGTCACCACATGATGTGCTGACCCCGTCGCCGCCGCCTGCCACAGCGCAGGCGAAGGCG
CCGGCAGCCAGTTCCTGCCTTCTCGACACCTTGAAGTGCGACGGCGCCGGGTGGGGCGTG
AGGCGCCGTGTCAGGTACATTGGTCGCCACCACGATGCTTCCAAGGAGGCCAGCGCTGCC
AGCCTCGATGGCTACAACACAGAGGTCAGCGTCCAGGAGGAGCAGCAGCAGCGACTGCGG
CTTCGACTGCGGTTGCGACAACGCCGGGAGCAGGAAGACAACAAGAGCACTAGCAATGGC
AAGAGGAAGCGGGAGGAGGCAGAGAGCAGCATGGACAAGAGCAGAGCCGCCAGGAAGAAG
AAAGCCAAGACTTACAAGAGTCCCAAGAAGGTGGAGAAGAGGCGCGTCGTGGAGGCTAAA
GACGGCGACCCTCGGCGCGGCAAGGACCGGTGGTCGGCCGAGCGGTACGCAGCGGCGGAG
AGGAGCCTGCTGGATATAATGCGCTCCCATGGTGCCTGCTTCGGTGCGCCGGTGATGCGG
CAGGCTCTGCGGGAGGAAGCCCGCAAGCATATCGGTGACACCGGCCTCCTTGACCACCTG
CTCAAGCACATGGCCGGCAGGGTACCGGAAGGCAGCGCGGACCGGTTCCGTCGCCGGCAC
AATGCGGATGGTGCCATGGAGTACTGGCTGGAGCCGGCGGAGCTTGCCGAGGTACGGCGG
CTGGCTGGAGTGTCTGATCCATACTGGGTGCCGCCACCTGGGTGGAAGCCAGGTGATGAC
GTGTCCGCAGTCGCCGGTGACCTCCTGGTCAAGAAGAAGGTGGAAGAGCTCGCTGAGGAG
GTTGATGGTGTAAAAAGGCACATCGAGCAGCTCAGTTCTAATTTGGTGCAGCTGGAGAAG
GAAACAAAATCTGAGGCAGAGCGATCTTACAGCTCTAGGAAGGAGAAGTATCAGAAGTTG
ATGAAGGCAAATGAAAAGCTCGAGAAACAGGTGTTATCTATGAAGGACATGTATGAGCAT
CTGGTTCAGAAAAAGGGTAAGCTGAAGAAGGAGGTGCTGTCCTTGAAGGATAAATACAAG
CTTGTGCTGGAGAAGAATGATAAACTGGAGGAACAGATGGCTAGTCTCTCCAGCTCCTTC
CTTTCTTTGAAGGAACAATTGCTGCTGCCAAGAAATGGAGATAATCTGAACATGGAAAGG
GAAAGGGTGGAAGTGACTTTGGGCAAGCAAGAAGGCCTTGTTCCCGGCGAACCACTGTAT
GTTGATGGTGGTGACCGGATCAGCCAGCAAGCAGATGCCACCGTCGTCCAAGTCGGCGAG
AAGAGGACGGCGAGGAAGAGCAGCTTCCGCATCTGCAAGCCACAGGGAACGTTCATGTGG
CCACACATGGCGTCTGGCACGAGCATGGCCATCAGTGGGGGAGGCAGCAGCAGCTGCCCT
GTCGCCTCCGGGCCAGAGCAGCTCCCTCGCAGCAGCAGCTGCCCCAGCATTGGGCCTGGT
GGCCTCCCGCCGTCGTCACGAGCCCCAGCCGAGGTGGTGGTCGCGTCGCCACTGGACGAG
CACGTGGCGTTCCGCGGGGGCTTCAACACGCCGCCCTCGGCATCGTCCACCAACGCCGCC
GCTGCCGCCAAGCTGCCTCCCCTGCCCAGCCCGACGTCACCTCTCCAGACACGGGCCCTG
TTCGCCGCTGGCTTCACTGTCCCGGCATTACACAACTTCTCCGGCCTCACCTTACGCCAT
GTGGACTCCTCGTCGCCGTCGTCCGCGCCATGCGGTGCTAGGGAGAAGATGGTGACCCTG
TTCGATGGAGACTGCCGGGGGATCAGCGTCGTGGGCACCGAGCTGGCACTGGCCACTCCG
TCCTACTGCTGA
SEQ ID NO: 59
SWI1; LOCUS# LOC_Os03g44760 Protein sequence
MDAEMAAPALAAAHLLDSPMRPQVSRYYSKKRGSSHSRNGKDDANHDESKNQSPGLPLSR
QSLSSSATHTYHTGGFYEIDHEKLPPKSPIHLKSIRVVKVSGYTSLDVTVSFPSLLALRS
FFSSSPRSCTGPELDERFVMSSNHAARILRRRVAEEELAGDVMHQDSFWLVKPCLYDESA
SSPHDVLTPSPPPATAQAKAPAASSCLLDTLKCDGAGWGVRRRVRYIGRHHDASKEASAA
SLDGYNTEVSVQEEQQQRLRLRLRLRQRREQEDNKSTSNGKRKREEAESSMDKSRAARKK
KAKTYKSPKKVEKRRVVEAKDGDPRRGKDRWSAERYAAAERSLLDIMRSHGACFGAPVMR
QALREEARKHIGDTGLLDHLLKHMAGRVPEGSADRFRRRHNADGAMEYWLEPAELAEVRR
LAGVSDPYWVPPPGWKPGDDVSAVAGDLLVKKKVEELAEEVDGVKRHIEQLSSNLVQLEK
ETKSEAERSYSSRKEKYQKLMKANEKLEKQVLSMKDMYEHLVQKKGKLKKEVLSLKDKYK
LVLEKNDKLEEQMASLSSSELSLKEQLLLPRNGDNLNMERERVEVTLGKQEGLVPGEPLY
VDGGDRISQQADATVVQVGEKRTARKSSFRICKPQGTEMWPHMASGTSMAISGGGSSSCP
VASGPEQLPRSSSCPSIGPGGLPPSSRAPAEVVVASPLDEHVAFRGGENTPPSASSTNAA
AAAKLPPLPSPTSPLQTRALFAAGFTVPALHNESGLTLRHVDSSSPSSAPCGAREKMVTL
FDGDCRGISVVGTELALATPSYC
SEQ ID NO: 60
ECS1 PROMOTER. Sense strand:
gagatttgggaaatgtgcaatttgggtttatctggttttgttgttttggttatttagttttgagtccggtttgaagaaatgcttcatagatatataa
atgtaaccagaaatataaataaatcataactaagtagtattatatttttgttaagcttaattatttcaattccaagtctttcctaagaatttgttgaa
aatttataatttacgttacactttgtaaaatcagaacgatccaattcacaagattaatgctacgactctgtttttttctttaaaaataaatcaataa
tcatctccactaaacctctcaataacttagcagtcttaatgaaatttaaagctaatctatcaatcacattctcaccacgtcgccaaactcgttg
ccgtttcaatcttttcaagttccctttcatgctgtttataaacccttgcactctttcactcacagactcactacaagtctacaccacaaacttac
caaatcatccaaaa
SEQ ID NO: 61
ECS2 PROMOTER. Sense strand:
acacgttatggaaagcaaagaataacaaaagtaatattcttacctatcttttttagttggaaaacttgcattgtgtaacgtattcaaacattttc
gaaatggtttattggtttttgtatataattaatttgggttaaactgatatattttatgagataaatatagaatctcatgtgcttatacaaagcaact
atattaattttgttaaccgtaagttacaaaaacagtggtcggaggaaatcaggaaaataaaaagagagaaaagagtctacacaatgggc
caattattataagtaaatgatagtcatgaaagcccatttcagaagaagatcttttggaaatgagaatagtgctctaggctcactggtccttttt
actattggtatagaaactgtcaaagcccaacaggtttaaactagcatttcaggcgctgtaattcttctgcagtttgtttgtataaacttggaat
atggatgggttaaacactgatatttcttcactcgttttgtctcacatactgttcgatgcttaacacaggtcttaaAAAGAAACTGG
GTTTGATGTCTCAAATCTACTCAAGAAAGAAAGATATCTTGAGTTTGCATCGAGA
CAGAAAAGAGTACGACTATACATAGCTGCTGGGGTAGTAGCCTGCAGAGAATAC
AGATTTTGAACCACGTACAAGGAACCAAATCAGTGTATGTATCTAACTATTAACC
TTGTGGTGTGATCTTGTCCTCTTAGGTATTGTGGAATCCTTGTAGGAAATGTCATG
GCAACTCATTAGTCATCTTGAACCAAATGAGATGATACATGATGGTCTCAAATTG
GACATGGTGGCACCTTTTGTTTCGTGAGTGGCTTTCAATTTATCTCCATAGAAATT
GTTTAATTTTCGTTATTGGTGCCTGTCAATAAAAATTACAAACATATGCAGAGCG
TTGGATTCGTGGATCGTTGAACATCCTATTGAGAGACAGGGCCAGCCTCCTAATT
GTATGACATCGTCTCTTTCACAATATACTCATTAAATGAGAGGTTGAGATTTGAC
TTATTTGCTTTATACAGCCTGCACAGTGTGGAAGACCCCTCTAAAGACTGAACTG
GGGACAGCAACAATGGGAATCTGAccatcctcatgacagtacctggaaagagtctcagaagcttcaagttcagt
acgcagcttgaccagtctttcagtcatatagccataggggttgaattagtgtccatcttcccattgtgattaacgttctgatttagcatgcac
cttcgaattaagtgaatctattaccatgtgaccaagccattgcattactaatataagcatatcacatttcccttttctccgtgccaactgaattt
gaattattttccctcaacttaatcacatgttttcctcacggccaaaagtactctcagtgttacatgattaccacaacaaatgatttaaactttga
acttttgaagttatcgagcaacatggcaaatcctggtcctatatgacataacatgagttcctctgcctattgtaaaattaggaaacacaaaa
ccaaaatgattatatctggtattatagtgtggtgtataacatatactcacacaagatatgctcttaagatgataaatgtctaatcttccaagtc
ccaattttgaaaacgttgatattaatttcccctcaaccccactagcctcaaattaaattagcagccttagtgtgaaattaaaagatagctaat
gaattgcatttcagactttcacctccccactcacgtagctataactccttaccgtttcaaatctcttcacttccccaattttgttgtgtataaaaa
cctcttctccacttcactctttccaccacaaactttctaaaactaatcaaca
```

1. A method of generating progeny of a subspecies plant cross, the method comprising,
providing F1 progeny of a subspecies plant cross;
transforming tissue of the F1 progeny with a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype;
selecting transformed cells from the tissue;
regenerating tissue from the transformed cells to form a transformed plant; and
collecting the selfed-seed from the regenerated plant.

2. The method of claim 1 wherein the tissue is embryonic tissue.

3. The method of claim 1, wherein the fertility rate of plants from the selfed-seed is
(1) at least 20% (e.g., at least 25, 30, 40, 50%) higher than the fertility rate of F1 plants, measured by production of F2 progeny, from a natural subspecies cross, wherein the fertility rate is the percent of ovules that produce viable seeds; or
(2) at least 80%, wherein the fertility rate is the percent of ovules that produce viable seeds.

4. (canceled)

5. The method of claim 1, wherein
(1) the subspecies are rice, barley, wheat, maize, sorghum, millet, oat, triticale, rye, fonio, or other cereal subspecies;
(2) the subspecies from the subspecies plant cross are indica and japonica, aus and japonica, or indica and aus;
(3) the first plant and the second plant are classified as different species;
(4) a first plant of the subspecies plant cross is a wild plant and a second plant of the subspecies plant cross is domesticated; or
(5) the Babyboom protein is at least 70, 80, 85, 90, 95, 98 or 100% identical to SEQ ID NO: 1, 2, 3, 4, 5, 6, 7, 8, 9, 10, 11, 12, 13, 14, 15, 16, 17, 18, 19, or 20.

6-9. (canceled)

10. The method of claim 1, wherein the MiME expression cassettes comprise at least one expression cassette encoding a gRNA targeting
(1) (i) OSD1 or an ortholog thereof, (ii) ATREC8 or an ortholog thereof, and (iii) ATSPO11 or an ortholog thereof, or
(2) (i) OSD1 or an ortholog thereof, (ii) REC8 or an ortholog thereof, and (iii) PAIR1 or an ortholog thereof.

11. (canceled)

12. Selfed-seed produced by the method of claim 1, or clonal progeny thereof.

13. A plant clone of an F1 progeny plant from a subspecies plant cross, the plant clone comprising a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype, wherein the plant clone produces clonal seed.

14. The plant clone of claim 13, wherein the Babyboom protein is at least 70, 80, 85, 90, 95, 98 or 100% identical to SEQ ID NO:1, 2, 3, 4, 5, 6, 7, 8, 9, 10, 11, 12, 13, 14, 15, 16, 17, 18, 19, or 20.

15. The plant clone of claim 13, wherein the MiME expression cassettes comprise at least one expression cassette encoding a gRNA targeting
(1) (i) OSD1 or an ortholog thereof, (ii) ATREC8 or an ortholog thereof, and (iii) ATSPO11 or an ortholog thereof; or
(2) (i) OSD1 or an ortholog thereof, (ii) REC8 or an ortholog thereof, and (iii) PAIR1 or an ortholog thereof.

16. (canceled)

17. The plant clone of claim 13, wherein the plant is a rice, barley, wheat, maize, sorghum, millet, oat, triticale, rye, fonio, or other cereal plant.

18. A method of generating a triploid fertile plant, the method comprising,
generating triploid embryonic tissue comprising a heterologous a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype;
regenerating tissue from the embryonic tissue to form a triploid plant; and
collecting selfed-seed from the regenerated triploid plant, wherein the selfed-seed is triploid.

19. The method of claim 18, wherein the generating comprises crossing:
(a) a diploid parent plant comprising a heterologous a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype with
(b) a haploid parent plant comprising a heterologous a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype.

20. The method of claim 18, wherein the Babyboom protein is at least 70, 80, 85, 90, 95, 98 or 100% identical to SEQ ID NO:1, 2, 3, 4, 5, 6, 7, 8, 9, 10, 11, 12, 13, 14, 15, 16, 17, 18, 19, or 20.

21. The method of claim 18, wherein the MiME expression cassettes comprise at least one expression cassette encoding a gRNA targeting
(1) (i) OSD1 or an ortholog thereof, (ii) ATREC8 or an ortholog thereof, and (iii) ATSPO11 or an ortholog thereof, or
(2) (i) OSD1 or an ortholog thereof, (ii) REC8 or an ortholog thereof, and (iii) PAIR1 or an ortholog thereof.

22. (canceled)

23. Triploid selfed-seed produced by the method of claim 18, or clonal triploid progeny thereof.

24. A triploid or higher ploidy fertile plant comprising a heterologous a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype, wherein the triploid or higher ploidy fertile plant produces selfed-seed that are of the same ploidy.

25. The triploid or higher ploidy fertile plant of claim 24, wherein the Babyboom protein is at least 70, 80, 85, 90, 95, 98 or 100% identical to SEQ ID NO:1, 2, 3, 4, 5, 6, 7, 8, 9, 10, 11, 12, 13, 14, 15, 16, 17, 18, 19, or 20.

26. The triploid or higher ploidy fertile plant of claim 24, wherein the MiME expression cassettes comprise at least one expression cassette encoding a gRNA targeting
(1) (i) OSD1 or an ortholog thereof, (ii) ATREC8 or an ortholog thereof, and (iii) ATSPO11 or an ortholog thereof; or
(2) (i) OSD1 or an ortholog thereof, (ii) REC8 or an ortholog thereof, and (iii) PAIR1 or an ortholog thereof.

27. (canceled)

28. A method of generating clonal progeny of a plant with at least 80% efficiency, the method comprising,
transforming tissue of the plant with a single nucleic acid construct comprising (i) a first expression cassette comprising an egg-cell expressing promoter operably linked to a polynucleotide encoding a Babyboom protein and (ii) a second expression cassette comprising a promoter operable linked to an RNA-guided nuclease and (iii) sufficient mitosis instead of meiosis (MiME) expression cassettes comprising a promoter operably linked to gRNAs to induce a MiME phenotype;
selecting transformed cells from the tissue;
regenerating tissue from the transformed cells to form a transformed plant; and
collecting the selfed-seed from the regenerated plant, wherein the selfed-seed display a frequency of parthenogenesis of at least 80%.

29. The method of claim 28, wherein
(1) the tissue is embryonic tissue;
(2) the plant is a rice, barley, wheat, maize, sorghum, millet, oat, triticale, rye, fonio, or other cereal plant; or
(3) the Babyboom protein is at least 70, 80, 85, 90, 95, 98 or 100% identical to SEQ ID NO: 1, 2, 3, 4, 5, 6, 7, 8, 9, 10, 11, 12, 13, 14, 15, 16, 17, 18, 19, or 20.

30-31. (canceled)

32. The method of claim 28, wherein the MiME expression cassettes comprise at least one expression cassette encoding a gRNA targeting
(1) (i) OSD1 or an ortholog thereof, (ii) ATREC8 or an ortholog thereof, and (iii) ATSPO11 or an ortholog thereof; or
(2) (i) OSD1 or an ortholog thereof, (ii) REC8 or an ortholog thereof, and (iii) PAIR1 or an ortholog thereof.

33. (canceled)

34. The method of claim 28, wherein the plant is
(1) a hybrid plant;
(2) an F1 of a subspecies cross;
(3) an F1 of an inter-species cross; or
(4) a triploid or higher ploidy plant.

35-37. (canceled)
